SEQUENCE LISTING (41 sequences)

SEQ ID NO: 1 - DNA, 25904 bp, unidentified
  source 1..25904: mol_type = genomic DNA
atgggtgtctccagcgatacaacgagccatctgctcgaagtgaagacgccagctatggacgctggggcgtctcccaagacgaggaggcgacgggccttgtcgggcgcggaggcccagcaagagttcggagcatccgccgctgcgaatctactgcagcaaatccaagcagcatggtccaaagccctaggactacctacggagctcatcggcacaacacagtcgtttgccagtctcggcggagactcggtggcagctatgaaagttgtctccatattgagtctagctggcatacgtgtgctggtgaaagatgtgatgcgagggaaatcaattcaggggctcgcatcagctgccaagcaggtggcagcaccagccacaaccgccgcctcgcaaccaccgccacaatcccaaccagaaccgcactcgttgaaggaagtcgcgacttgcgagacgatcaacaacaaactcaacaacttgaagcttgctgacgagcgccaagagcggctcgatcatctagcattcaaacatggatccgatactgaggtggaggatatgtacccctgctcgccgacgcagctgggtatcctcatcgcccaagccaagaggccaggtgcttatgtgatgcgcatcctgtttgaactttccatatgcaagggtaacgacaaagtcgatgtcagacgcctccagaaagcttggcaatctgttgtcgagcgtcatgcatcgctgcgcaccgtcttcgtcgaaggcaacacggccgaatccccgtttgagcaaatcgtcattcgacgcgtcaaggcggactttcgcgtcttgcgcatcatgaccgatcatgccagccaggaccgtcttgtatccctgggaaaccacggcggcgccctggccgagttcggtgaactgggtcatcgtcttaccgtcattgccaccccgtctggcaagcactacatccgcctggatatcagccatgctgtcaccgacggtggatcggtcgacatcatgattcgtgatctgaaactcgcgtatggtggaaagttggccaaaggccctggcccgacgtaccgatcgtttatcgacgaaatcctgcaccaaccctcagcccatggtctgcagtactggtcagagtaccttcgcggtgccgagacatgccagttccccactctcctcgacggagacatgtcctcatccccagaaagtgaactaatgtctgcagaagtcaatctttcatctctcacaaaagagattgcagacttctgccgcctccacgaggtgacccttttccacttgttccaggtttgttgggctgttgtcttgcgcgcatatctgaacaaagacgatgtctgtttcggatacacggcctcaggccgcgatgcaccaatcgactccattgagtccatcaccggagctcttgtgagcaccatgctctgccgtgtccaatttgatacgtccaaatcggttacaagcctcatcaaggaggtagcagagcgcaccacagaaagtctcgactttcaacatcattctcttgccgacgtacaaaaggccgctgatcttccaaaagacctcgttttcaatactgtaataggatttcagaccgctctatcgttggactcggatgacacccaagctggccttggggtcaaacagctcagcgtgtattcacctacagaggtacgcaaaacccgcctgatttcttttagatctggttgctaaatgtgacaatagtacgatatagctgtcatcgttgacacggcatccgaagccccttcggtgaatttggactattggacacataggatatcaccagcgcaagccgagatactcgccgatacacttgcccaagtaataacggccgtggtttctcatagcgactacaagattgggcaattggacgtcgtttcaccaaaccaataccagcaaatggttgactggaatatcgattcaactctggaacacggcaacttttctcaaggtacgtcaaccctacatggcatcttccgtgaacgagcgttattatgccctgcggcacccgccgtggtagcccacgacgggcaactgacttatagccaactcgacacactgtcaactcaactttctcatcatttggtatctcgtggtgtggaagtcgagacgctagtgccctactgtttttccaagtcgctatatacggtggtggctattttgggaatactgaaagcagggggtgctgcggttccactagatcccaatcaccccccagatcgcctcgacaccatccttgacgatacgaaggctgccgttattgtcacctccccagaacacagtcatctgttccagcacagacccgccgcaattgtttccgttgatgacgcctttcttcgtctgctcccagaggtctgggaagcccctaacgctgccccgacggccgactcggcagcattggttgtgtacacatcgggtagcactggtgagctatacccaaataccaccgattattagctgtgactaactcgaaccaggtgtccccaagggagtcgtgtcgattcatcgagcagtggctacgaattgccaagcttatggcaccgccctccagatgaatgagaagtcccgcgtgcttcagtttgcctctcacactttcgatgctagcatattcgatatgtttgccgcccttcttctcgggggttgtgtgtgcattccgtccgaggaggataggatgaacgatttgcctggatttatcgagcgcacaagctgcaactggattcttgctacgtcaacggtcatgagcatgattacaccgcaacaggcaccaggtctcgaggtccttgtgctgggaggagagccactctcccggacagtgcttgagatgtgggcaccttttgctaccgtattcaacgcgtacggcccggccgagagtgccatcatgtctacgtgctcaccgaagctcactgctgactcccaaccatctctgatcggaaaagctgtgacagggcctgtgtggatcacagacacgtcagatcacgataggctgatgcccatcggatgcattggggagatcagcgtccaaggtccaggagtgtcgcgaggctacctgaacgcgcccgacaaaacctcaaaggtgtttttcgacagtcccgtatggtcgcatccttcgtttgagccgtattctcgattatacagaactggcgatctgggccgcctcaacagcgatggcttcgttgaaattctgggtcgaaaggacaaccaagtcaagttgcggggtcagcggctagagttgggcgaggtggagtaccagatcgcatcgcacgatcaggtcaagcagtgcactgtcgtgaagggcggaaagggtctctgcaaggagcgtcttgtgggtcttatctctctccacggagagccaaagtccatggacactcaaatacgatttgcggaggacaacgacaatgccaaagcccaggtagtctccgaaatccgagaaaagctggctggtgcgctgcccacttacatgatccctacgctttgggctgtcctgcaagagctgccaatcaccacctcgggcaagatcttcaccaagggcctccaacaatgggttgatgagatgagccaggaggccttcgtccgagtcactcgatcatccaaggacggcgagagcgaaaaggatgaagaatcgaggacgaagatgtcggggactgaacaaaagatccagaagatctgggcccgtgttctcaaccttcccatcgaagcgatcacgcggtcaaacacgttcttgtcagtaggtggagattcggtaactgcaatgcgtgttatgtcactctgcagagccgaaagactcctggtcatagttcaggaggtattgcgcgaccagacgctcgctagcttggcaacttgtgctaagactatgggttcgaccaccgcatctgacgacgtcgaagatatatatgacacgccggttggattgagcccaatccagaacctcttcttcgcgacacagggtgtctcgaaccgatggaaccagagcttcatgctcgagttacgaagacctattccactggagagccttcgtggcgcattgaattctctcgtcaagcgacactccatgttgcgtgcccgctttcaaaagacatcgacagggacatggacgcaacagatcccccgaccagatgacggtgtagttcatcagtgcttcaccttcgacactactgatatatcaggagatgtctcaatggtagagcatgcgaacctggtccagtctcgactcaacattgaagatggacgtgttttcacagcaacactgatgagcaagggtgaagagcagtacctttacatggttgcccatcatctggtcattgacctcgtttcctggcggatcatcatccaggacctcgaagatatgctatcgaacaccgcgctcagcaatactctggcgacgcccttccttcagcttactaaagagcgtattgccaagttgggcgacgcaactgcacaccaagatcagagtcgaaatgtcaactctgcggcaaactggagctactgggattttgccgaacagcccaacatcgaagaggatgcccgtaagctgagcttttcgctcgaccaagatgtgtctgggcagcttcttggcgcttgcaacaatgtacttcggacagaacctaccgatctgtttgtcgcggcccttctcaattcgttcgcagaggtcttcacagatcgcaacgtgcctgatgtattcgtagaaggccacggtcgagaccatgaagcgttccaggaaatggatgcgaccggtactgtgggctggttcacagccatttggcctctaaccattccgattacatgggcggggcagcatgtggatctcacgcagacgattcgagccaccaaagacacaagaaggggtcaagagggccctggcaagaccttcctggattctgtcatagccccaggctcatcggatggcgtgccaatggaagtcatctttaactacgaaggacaatatcaacaattggagagttcggatggcctattccggatgctacctctcccagcccctgatatggcttctgatgtgcagcgcactgccttgtttgagatcaccaccgtggttaagggaggccaggtgatgtttgaattcttattcaacgccaagatgggccgtcaagaccagattggcgagtggatcaagaagactgaacttaccttaaccaagcttgccaccgagctgcccaccagatcaaagatgtatagcttgtccgacttccctctcctccaacttgattacgacaatctgcaaacttttacccaggggctagaagttcgacttcaacctttgcgccaacgggatagctcgtttgaaattgaagaggcctacccctgtacaggtatacaggaaggtctactcctatcgcaagggaagcagtcgggaaactatgcagtgcacgagacactggaggttgtttcagcttcaggaacacctattgatgcctcgcgtctaagagacgcttggctccaagtcgtcagtcgacaccctgtgttgcgaaccgtctttgtggaccacccgacggatacggggtatcatgtccaagtggttcttcctcgtgtgatcccggaaatcaacatttcaacgtcccaatcggtcgacgaagctctgttgctgtggaagaacctggggaagaacgattatcccgctgggcaactcatgcaccgctttagcatctgcagcactgctgggggtcgggctctggtcagattcgacttcagccactgtatccttgatgcccagtcttgccaggtgctattcaaggatttgagtcaggcctacgctggaacgctcgacatggacggaagtgtgccacgattcagtcaagttctgcagcatatgcaggggcgtgatcaggcttccgacctgacattttggcaggctgaacttactggcgcggaaccaagcctccttccgaatctttccgactcatctgacgagaccgagcttcatatccagcccctcgaacttggtatatctgccgacgccgtgcacaccttctgcaagcagcacagcgtgagcccgtttgcgatcttgcaggtggcctgggcccgggttctcagcgcctacacggcgatggacaaggtgtcctttgggtacctcagctccggccgcaatgccccaatcaatgggattgaggatatcgccggggttctcgtcaacatgcttgtcacgcatgcgaaggtcgactatagtgtagccgcaattgatgcggcgaaggacatgcatcgcagctacaccgaaagcctggaccaccagtactgctcgctgacagaagtccagcatgctcttgagctcggcggacagccgttgttcaacactgttctcgacttccaacgacctcccgaggaagatgatttggacacagctatctccttacgtcctctggactttcacgatcctacagagttcgctctcacactacactttgagctcaatcataaatctttctctggctacatgtctcactgggcctcgacattaagccaatcatatgcccagagtattggagaggccttggtactggccctacagactatcatggcaaagcccgacatcaaactcggtagcattgacctcatcagcaacaagactctggacaagtacatcgacatcaacactccggcgcccgttgccaagtctgagtgtcttcatgacaccttcagacgccgtgtacaaatggtcccagatgccccagccgtcttctccactgaaatgagtctcacatatgctgagctggatctcctatcaaccaagctggcaaatgagctcttgggcatggacgtgggtcccgagacgaacattctgctgtgctttgagaaatctgcgtggacgattgtgtccatgatggcagtatggaaagctggcggctgtgttgtcgcacttgaccctacacaccctgcttcccgcctaaagggcatagctgaagacgtggctgctcgcataatcctggtctcgccagctactattgacacttgtcgcgaaagcctcgacgcgcagatcagattcatcgttgtcgactcaatgaacttgagcgcaatgattgatgagcggcccaatgtcacgcccgtgaagccggagaatgctgcttacatcaatttcacaagtggcacaaccggcaaaccgaagggtgtggtcattgagcaccaggccattgcaagtaacgtagagcccttgactaaagcggcagacatcaacaacacgacacgagtcctgcagttctcatcgtatgcttgggatgctttctactgcgagaccatcatgccccttctgtctggtggatgtatctgcgttgccaacgaccacgaaaggtcctacgatatcgccgagttcatccgacgcgctaactgcacctgggcgctgtttacaccatcattcgcaaggctcttgacgccttctgacgtgccaaacctccgcaccctactcttgggcggtgaagccatgtcagctgatgatgtgaccacttggtctgagtccgtggtcctcaagaacgcgtatggcccatgcgagtgttctatcgtcgctcttatgcaccccaggattgacgcagcccgcagtgggtcgaacctgggacatcgcttgggccagaccctttgggttgtcgaccctagaaacccacagagacttgcgccagtaggttctccaggtgaactgctcctgggtggtcctcctgtggcgcgagggtatatcaatgacccagcaaagactgccgcggctttcatctcagctcctgcatggttcacatctaacgctgtcctgggcgagcaaggcttcacctctcggttctactgcacgggggatcttgttcgtctcaatgcagatggaaccctcaacttcctcggacggaaggatggagaccaagtgaagctgcgcggccagcgcatggaactcggcgaggttgagcaatgcatcatgacaagtacgtcgacattgaagcaagtagccgccgcggtgatctcccgcaccggccatgccggggacagacagcttgttgccttcgtcaacacgtcggactctaccgacttgaacagcaagccacagacattgcccatgtcggatgagttgctgaagagcctccaggcgctgacgacaaccatggcgaccaaggtaccctctttcatgataccttccatgttcatcccagttaatcgcataccactgagtgcatcgaccaagatcgaccgaaaggctttgaccgagctggcttgccagatccacgaatccagtgtcgctcgttatgcactgactggactttccgaggcttgccccccgacaacgcccatggagataagtcttcacaccctctttgctacagcattgggcgtcgagaccacgtcgctttccatcgccgacaacttcttccaccgtggaggcgactctatcaaggcgatcaagctggtggcggctgcgcgacttgcgaatgtcaatatgacagttgtcgacgtcttcaatcacccaactgttagagagcttgctgctgtggccaacttcatcaactccacaccagacgacccattacgaaatgatagtctcgaagtcttcgtgaaggagatgaagaccgaggtcaccaatgtcgcaattgaagcacacaagttgaaagcgggtcagattcaagatgtgtatccctgcaccgagctgcaggacgcgatgttgtcgataacactgggccagccaggcgcctatgtcctgcaacttgtgacctcaatctccgaagatgcggatcttgaagcctacattgacgcttggaaggccgtgagaaggaataatgacatcttgcggaccagaattgtgagcgcatttgggaagcgctaccaggtggtgtacaatgaccgcatgataatcaagaatgaggagtctcttgacgattatctcgcttctgacaagcagaagcctatggcatatggcgacgcacttgctcgcttcgccctgatctccgaaggagaaaagcattacctagtgtgcaccatgcatcactcaatctacgatggatggtccgtttcactcatcatggatcagctctccgaggcctatcagtggaagccccttcagaagccacagccattctctgaatacgcttggcacgttttcaaccaagatcgcgttgccgcggaacgattctggaccgctgaattggccggcgccacgacatgcgacttccctcgcatgccatcacctacctaccaagccaacgcccgtcagaacgcagcccgaactatggtccttcaacacagccagtcagacatccccctcgcagcaatcatcaaggcagcctgggcgcttctattgggtcaacactccggctcgcgtgatgtcgtctttgcgacaactaacgctggtcgcaacttggatctaccaggcattgctgacatgatcggcccaactattgccacagtacctgtgcgtgttcgctacgattcatcagatagcatcaaagacatggtgaccaaggtgcaccaggggtctctttccatggttccatttgagtatcttgggctgcagcacatccgacatctggacgcctcttgcgaagccgcttgccaattgcgaagcttgctggtcgtccagccaggtgctctcgacgatgctgacattctgcccatccctggtgtcgagaatgtaccagtcgacacaggcaacttctttacgcagcccttggtggtggagtgtaccttgggaaccaacaaagtgaagctctcggctacctacgatgatcatgtcttgagctccgcagagacggatcgcttgcttgctcagcttgagcacttcatccggcaattgagcatgaatatgacaaaaactgtgggcgatatggaccttgtctgccccgaggacaggctcgacctcaactcttggaacgccaatgagccgcccaaggtgaactcgtgcgtctgggatgtgattgcgaagaagacacaagaaacaccaactgccctcgccatctcatcttgggacggtgaaatgacttatgatgaattagacaggcattcgacacgccttgctgctcatctctgtcattcgcttggggtcaaatgtgagacactcgtcccgttggctttcgagaagtcggcatgggctatagtggcgatgctagcggtcgtgaaatccggaggtgcgttcgtatttcttgacgacaaattgccaccagcacgacagcgattcgtggtgacatcaacccagtcccgattcgtcctcagctcgtccaagaatctccccggctggaaccactctcaatggcggagcgtcgaggtcagcgaagcagccataggacagctgcctgaccaagttgcgcccacgcccagctaccagccggacagcatgttatatgccatatatacatcaggtagtactggtaccccaaagggctgtgttgtggagcacgccgcattcctgagcagtgccgctagctacaccaagctcctgaagatgggtccagacacccggtcgcttctgttctcctccttcgcctttgacgtcagcctgatggagtctcttgcggctctcacagtgggtggctgcatctgcatcgccagtgaacaggcatatgaggcaggtgtcgccagcatgattcaggaagccaagcctaactgggcatctctcaccccatcagtggcaagattgatggagcccagagatgtgccttctctcaagacccttgccctcgtaggggagcctttgagccaggaagatgtcaagacatgggctggaagcctccagcttatgaatggttatggcccgacagaatgcgctgttctctgcctcattaatgccaacatgacgacagagaccaaggctacaaataccggctatggcagtggtgggcttacttgggtcgtcgatcccaacgaccctaacattctcatgccaattggctgccccggtgagctcctcatgcaaggtccgatcctagcccgcggctaccttgaccttcctgaggtgacagcatccgcattcattaccggcctcaagtggtcgccccaggatcggttctacaagactggtgacattgtgcgatacagccccgacggatccatccagtgtttcggccgcaaggaccaacagattaaagtccgtggacagagagtcgaactgggtgagattgaggagaacatcgatacactcgactctgtcagccaggcatgtgtggtgttcccaaagtcagggccatacgagaagaagatcgccgctgtcgttgtgctcgaggagttcgcagctcttgactcagctcctctccacctcatttcagccaagcaacaaaaattcgcaggcatcttggtcaaccaaatgcgtgatgccctatcagagaacctgcctagctacatggtcccagaagcctggatcctcgttaccgctctccccatctcaatttcgggtaagagagaccgctcaaccatcggccgatgggtaacggaaatcgacacaaagcaacaagagcagattgacaacctcctcaccccaaccacgattgagtttgccaagacagacgacgagaagaggctacattccattgtgactgaggtgctcaatattgacccttcgcgggcgggtatcaaccagtcattcatgagcttgggtggcgactctatcagcgcgatgcggttaatagccaagtgtaggagccaaggtatcacactcagcctcaagaagttgatgagcagagccagtctttcgcagttggctacttccatgactgcgaatggtagccaaagtgctacagctcaagtcgcctcggacgacaccgggaaaccattcgacctcacaccaattcagcaacactatgtggatcttcacgctcaatgcccgcctctggatacggaaacagagtttaccggccggcgtcgattccaccaaagcttcttcatgaagctcacaaacacagtcagcgcttcgaatgtgactgtcgcggttcgaggacttgttgagcggcacccaatgctgcaagccacattcggacaagattcgaagggtcggtggcaacagactgtaggcagggctcccagtacagttgattgcatctgggtcagcgacgcccaaaccatgcaagcagcgtcagcaacactcgcttccgccaacagcctcgacgtgatcaacggtcccgtattccgtgcagtcttcttttcgaatgatgttgacggtcaaggacaattgcttttcctgacagcgcaccatctggtgattgacttggtgtcatggagaatcgtgctagaggagctagaacagagccttcgtggccagccactacctgagccaactggattgagcttccaatcatggatgaagctccaagccgaccatgcccggctcaacctcagcccaaaatcggccttgccatttgacgtgcatgccagtaacctctcttactggggtatctcgacaccgtctgagaatacttacggcatgaccaagaccgaaagcttcaacttgagcaaggatcttacctctctactgatcggccaatgtcatgaagtacttcgcaccgatgttgtggatgtactcttggcgtccgcgttccgggctttcaccgacatcttcactgagcgagatggtcccacagtgtttgtcgaaggacatggccgggagccttgggatgagtctatcgacattggttcgactgtcgggtggttcactgccctgtgccccgtccacgtacccaagaaacccgaagacacaccccttgagaccctgcgagaggtgaaggatgtgcgccgcagcattccccagaacggatgggcgtacttcgcgtcgaggtacttgaatgctgatggccgagctgccttccaagacgacttgatggcggaaatcatcttcaactacgctggacaatttcaacaactggaggctgacaaccagttgctcgtccctttcgatggcgaactgcctgaagaagcatctgatgttggccctgacacgccgcgatttgctttgatcgaagtgtccgctgtcattcggaatggtcaattgcagatgtcgttcatcttgaacgatcgcatgaagcacgtcgaccgccttggagaatgggtgcgctcggttcagaagaccctcgagactcttatcagcagtctttgcgaccaaccgcagaaatcggtaccgacgctgagcgacttcccactcctacgtaccacccatggcaggcttgatgatcagatatctgaagcatgcgcccggttcggtgcttcggattggactgaaatggaggatatgttcccttgttcaccacttcagaccgcaatgttactgaacaactctatgaatgccgaacaataccgctcatgcgtgatcttcaacataacttcccctacaccaatcaccgccgaggctctgagcgctggttggagcaaggtcgttgcccaccacccttcgctccgaactgtgttcatggaaaccaagacagataatggggcatttgatcaggttgtcttcaagaacctacaacctcaaatcaacgtcctagatatacagggcgatcttgccctcgctactttgaaagcccgcggcccggcggattggtcaaatggcgctcctcatgtcttgaccgtcattccgacatcagctggcgtgctatgcaagcttgagattagtcatgccctgatggatggcgcgtcaatggatctgctatttggcgacctcgtttcggctttggaagacaatctggatgctgccccgagcccttcttaccacggctacattgcgtatctccagaatcgagatgccagtgattccttgacacattggatgagctacctcaaaggcgctaacccttgtctcatctctcctatcaagtctgttacgcagcctgtcgagatcaacgaggtaacttcaatcgaggtaccaatagttgatggcaccgcacaagccctcaacaactttgtcgacgcccatggaattacacaggcgtcggttatccaggctgcctgggcgatggttctgcgtcgctacacccagtcagaagatgtttcgtttggttacgcagttgcaggccgcaatttgccaattgaaggcatcgagaagaccgttggccccttcatcaacgtccttccgtgcagacttgacatgagcgacgacaagctggatatccgtggcttggccaacaaggtccagcaagacttcttcgagagtctccctttccagcatacctcgcttgcggagatgcagcatgagttgcaacttccgacccgtgcgttgttcgacacggtggtgtcaattcagcgttacaatgaggcgtccatgatctcttcaaacggccttcaaatgaatcttctgcacagccgcgatccaacagaatttactctgactctcaacgctgcgtactcgactgacagcttgggctttgttgtgagatactctccgcaaaacggtgttacaagctctagggcggaaagcatcgcgcacacgttccaagctgccctgagtgcgatccttgccaatccagagcagacacttcggaacgttgacctggtcagcaagcatgaccgtgacattattgaccaatggaatgcccatgtacctgaagcagtcgatcaatgcgtgcaagcaagcattaagctgcgtgcagtttcccaacctgcagctgcagcggtatctgcatgggatggagagctctcttaccaagagctggatgatcagacaaaccgtctcgcgggacacctagtacgcaagggtgtcaaagccggcaccattctgccgttctgcttcgagaaatcgatttggaccactgtggcagttctgtctatcttgaagacaggcgcttcgtttctccctttgaacatcaacgaccccgaagagcgtgtcaataccctcctccaggaggctaatgcacaccttgttctcgcgtcgcctagccaatctcggaagtttgaaggagttgtgaatactctggtcatctctgaatcattcattgcggctctcccaccaactagcgataatgagttgcccacaattgccccgagtagcgttgcctatatcctattcacgtcaggttcaactggcaagcctaagggtgttgttattgagcaccggtcgcttgctagtaacatcgcagagcatgcgcctggtctcggactcaattgctccacgcgtgcgctccaattcgcgtcatactctttcgatgcatcgattacagaaactttggcgacgttggtgtcgggaggctgtgtctgtgtcccgtcggaggaggaaaagacggccgacatcgaaggcgcaatgcgacgcatgcgagtcaacttctcgatgctcacgccttcagtggtacaactgttgtcgcctgagaacctcccatctttggaaacactggcgcttgttggagaagctgtaccaaagagcttgatcgagaagtggtccaagaaactcaatcttcttgttggctacgggccgaccgagacggcagtttttgcgtcgattggaagtctacagggccccaacgacgctgggctgattggcaaagctattggcactcaaacgtgggtggtcgaccccgctaaccatgacttactggcgcctatcggttcactgggcgaactcgttctcagtggcccgacgctagccagggggtacctaaacagtcccgacaagacggaacaggccttcaagacaaccccggcctgggctacccctggtataatcaatcgcgtttacaagactggtgatttggtatactatcgcgacgatggcaatctcatgtacgccggccgtattgacggtcaagtcaagatccatggtcaacgtatggaagtcggcgaagtcgagcaccagatccttgagtgccttccagcgcattcggagacagctgttgctgtgctgaaatccgacaaaacctcaattcagcgcttgatggccttcataagcatcccccagatcgaggctgcgcctgcaccctctccgcgactcgccatcttctctgacaaattaagagatgtgcttttgaagttgcagaaggatctgtccgagaaggtcgccacttacatggtcccctctttgtatattcccatgtcatctctgcccaaatcagcagctggcaaggccgaccgtaaagctatgttgctgctcgccgggtcactatcccaggatcagatcgatctgtactccctacgagcctctggtggacaggaagtttcgaccaaggcggagaaatttgtgcagtccgtctgggcatccatgctggggattccggctgataccatatcatcggatgaccatttcttcaaactaggaggcgactccttatctgccatgaaggttgcatctcaaatgcgcagcgagaatgtctttatctccgtcactgagctcttttcgcatccagttttgtcggcgctggccacactcgtggagtcaaagacgggagcggcggaggtcgtatcggtaagcgacgtatcgcccttcgagcttctgtcagacaagcaatgcgtcatcgacaggatcgagaagaccgtgggagtatccatgcatactgtcgaggatgcgtacccagcaacgcctctccaggaagcgatgttcgcaatcacacaaacgagcgattccgcttacgtcaatcgcatggtcttccagattccttcgcacatcgacttgatccgattcaagaaggcctgggaagccgtgtatgctgtcacgccaattctcagaacagcgctcgtcaataccgaggagcatggtacttgccaggttgtatacaaccgtcctgtgccttggtttgaggctgaatgcaacctcgcaagctacttgtacacggattctcaggggcccatctcggaaggcgatgtcctcaccagatacgcaatcatccatgatggtcccgcacattggttcgtctggacagctcaccatgcggcctacgatggctggtcagctggtgcacttttcgatttggtccacgcagcttatgagagtggtgtcgctgttcgaacaccatcttacagcaacttcatccgatacgtctgcaactcgaacgtgaatgatgtcgatgaatactggagcaccacacttgatggttatacccgaacggatttccctccaattccacacccagattaccgagccagcagtgaccaatcagtcgttcactccattcaactacccgccaccagacagaacgccgatatcacccaagcaaccatcgttcgagccgcttgggctttggtggttggcaagcagagtggaactactgatgtcgtcttcggtatgacccagaatggacggaacacacctgtcgcaggcattgaagagatgcaaggaccaaccatcgcaacagtccccgcacgtgttcgaattgacatctcaaagagcatatcaacatacctgcaagagatgcagcagcaagcagccggctcgatcccttatgagcaagccggactccagaacatccgcactttgaaccaaggagcagaagaggcctgcgacttcaacaacctgctcgtcattcaacccaagggcgaagcgggcggagaagtctggctagagcctgttactggagaactgaacaaattcgacaatttcccactgactattgagtgtgcgattggaaaggagggacatatggagctcttggcccacctcgactcccatctgctctccaaactccaggttgagcgcatcctcttccaatttagccatgtgatccgtcagctttctgaggccaacactgagactctactctctgatgttgagttcttcactcctgaggatcgtgctaccatcttcggtgaatggaactccaacatcccaggcactgttgaagcatctctacaagacagattcgaagagaatgcccgcgaatatccacagaagcaagcaatttactcttgggatgcgttaatgacatattcggagctcgatcagatgtcaacacatcttgcacacaagcttatcgatgcgggtgtgaaacctggggagttcgttccactctgtttcgagaagtcagcgtgggccgttgtttctatgctggcggcgatgaaggctggagcagcctttgtgtcaattgatcccgagcatccagagaatcgtcgtcaggatataatcgaccagaccaatgcccaagtcatgctagcttccgggacagccccacctctatcggtcgaccagaccttggtcgtctcggccgatacactatccggctatcgacaaactaaggactcatctacattgccccccaaagaccctagcaggcttgcctaccttatctttaccagcggtagcactggccgcagcaagggggttatgatgtcacaccgagctgtctgctctagtgctcagatgttagacttggaaggtcttggtttcgcccgggactccagagtgtaccagttcccctcttacgtcttcgacgcatctatcttcaacatcttcgggactctgtctgcaggagccacgctggtggtaccagctgacgaagacaggcttggtgccaacctagccaagtcaatgcgtgacgcggaagtcaccaaagccatcataaccccgaccgtcagtcggttactccaaccagaggatgtaccaactttgcgcaccctcaaactgggaggcgatgctgtataccaagaggatgtcgacaagtgggcttcgaaagtacacatgctgcagggttacggcccagcagaagccactgtctttgtcacctggacagagataactccccaaagcgtacctaacaatgtcggacctgccagcagatccaggctttgggtggtggaccccgacaaccacgacaagctgacacccataggcatggttggcgaaatgctcattgagggccccaccctcgccaacgggtacctgaatgacgaagagaaaaccgctgcggcattcattacaagcccggcatggtgtcataatgagtcaagacgattttacaagacgggagacctagtccgctatgccgataatggttcgttcttgttcatcggtcgcaaagatgcccaagtcaaaatcaatggccagagagtcgagctcggtgagatcgagaaccatctcttcaacatgttgccatcatcccaattcgtcgctctcgttccaaaagccggtccctgcaagaacgttctcactgtactagtggccgcgacaaccgctgactcagtcaagagacttgagtttatcgaagatgcctccacgaaggagtctttgagcaaggctgtctccgaactgcgtgaagccatgccttcccgtgtccccaagtacatggtaccacgtacatggttcgcggtcagcaggctacctcttacttcgtcgggcaaagtcgaccgcaagaatatccagcgctggctggaagagcttgaccccgacgtcgcacggatggaaacactttctcttaagcacgatgccgccgctacaatacccacgaccaagacggagagtgttgtacagcaggctattggccgagtcctcaatctcgcacctgagaacatcccgctcgacaagaccttcatggccttgggaggtgactcgatcactggaatgcaactggtatcccgacttcaatcgtcaagcatcgtgctcagcgtgcgacagctgctccaaagccataccatccgacaccttgcgtcgcttgcacaacccgaaggcgaacgggtggagctagcggatgaagtggttggcgtgcctttcgatttatcgccaattcagaccatgctgattgacaccatcttgcccgatgcaatggcacctcggcgtttcaaccaaagcttccttgtccgcttgagtcacgaggtggatacagggaaattgcgagatgccttcacttaccttgtatctcgccattccatgttgaggtcacgttttgagaagacctcagatggccgctggagccagagaataatgccagagtcagacagcgcataccagctctcaatccatggaacgtctcatgatctgggcaagatcatggcggatagccagagcggcttagatgtcttccaaggaccagtttttgcaggtgatgtgtgcgccgacaatgaaggcaagcaagtcttgttcatggtcgcacatcatcttgccgttgatttggtatcatggcgtatcatccttaccgacctcgaggagtacctgacaacagacaacttcaaggagaaacacaagccgttgtcgttccagacatggctatcactgcaacaaaagaaggcggaggatattcgaccggacactgctcttccctttaccccaccacaagcgcctatcaactactggggggcactcactgaatccaacacctttagcgccgtggaggaacagtcgttcacactcggtgcggatctaacatcgcacctactcaaggactgcagccaagtgctaggaactgacacggttgatatcctcatcggcgccttgacccatgcattctcgaaaacgttccgggatcgcgacctgccgactgtcttcaccgaataccacggtcgcgaatcttgggatgattcaattgatatctcatctaccgtgggttggttcaccaccctggcgcctgtgcatgtcactagccatggcgacgatggtatcaaacctttcgtccggaaagtgaaggattgtcggcgaaagaccccaatgaacgggtggtcttactttgcttcgcgcttcttgagccccgaaggcagagagcgattcgcccagagcggacccatggaaattgtgctgaacttcgtcggacagtaccagcaactcgaacgagccgaaggactcttcgagatggtccctcgcgatgactttggctcggacaccgtcgacgtcagcccagatctggaacgctcctcaatcttcgagatttcgtctggcgtgatgaatggccatctgaatttctcaatgatgtggccacgaagtcttcgcaaccaaagccagattcgacaatggctgaccaataccgaacaatctctgatcgaagctatcaaagaccttcaaggtctcactagagagttgactgtcagcgacttccctctgctctctatggcagatgacgagttcgttagctttgtggaggactctttgccaaagcaccacatcgctgttggggacgttgaagatatttaccctgcgacaccactgcaagaagccttaatgatgtcacgaagtatcgactccgaattatacaccgtacacgccatctaccagctcagctgcaaggatggcgagcctgttgatacggagagactcaagaccgcctggaactccgtggtcagccgtcatcagagtctaagaactgtcttcgttggcgttgtgtccgaccacagtgcactcaatcaagtcgtactgaaggcgatgcctaatacgacagtggaattgacttgcgatactgacgaccaggccctggaaatgctcgaaaagccaaacacactgagcccaagtctagcctgggaggtagaaaccccagcgcagctgatcatctgccatgctatagcatccggcacaacttactgcaagatcgccatcaaccatgcggtcgccgacggtggctccaatggtatcctcatccgggacatcggcctggcctacgaaggcctcctctctgcagagcgtggtccgctatacagtcaatatgtctcttacctccagagccgcgacagacaagcaagcctcgactactggggcgaatatctgcacggtcttgaaccgtgcatggtcaatggtggcacacgctctgggaagaccaaggaactccgtgatcttgaagtcgagatcccacatcccgacaaattgagagctttctctgctgcgaggtctgtcacactcgccaacgtcttccaagcagcatgggccttagtactgcgcaacttcgccggcacagaccaagtctgctatggttacctcaacgccggtcgcgattcacccgtcgacgggattcgcgatgcggtcggactttttgtgaatatgctcgtgagccgtgtcgatttcaccgagaaccttacagcaggagagctagttaagcaggttcacaatgactacatcgacgccctcgatcaccagaactgttcactggccgacatgcaacggatcgccggccatccgctattcaacagcatcatgtcgtaccaggtcgcttcatcttcgtccgaggagacctctagcgatctcaactttgcccaagtcagcgctcatgaccccactgagtaccctcttagtatcagtgtcgcggtttccgacaagtcgatcttcgtcacccttagctactacacagatctggtttcagattttcaggcgcagagtgtggccaccagcttcgagaaggcgctcggcgggttcactgaagcaacactcgaccttcccgcgcgcaacattccaattctcggcgataaccacatctccaagatccaagagtggaacgctgaggggcccaaagcggttgaaggatgcattcaccagatattcgaggaccaagtcaaggcgcaccctgacgccatggcggtgtgttcaaccgagatcagctggacctacagccagttggatgtgctttccactcgcctggcacatcgcctcgccatcctgggtattggacccggcaccatagtcccgtactgcttcgctgcctccccatggacggtggtgatcatgatggctattctcaaatcgggaggtgcctgcgtggctctggatcccaaacatcccatcgatcgtctgcacggcatcatcgaagacgttgacgccgatattgtcattacttcgtctcgccacgcttccaagttcttaccggtggcggcggtattggcgctcggtccggaagaaattactcgcattggcaaaaccgcacccagacggcttggcacgttgccaatcgctcaaccaagtgacgtcgcagttgttcagttcactagtggcagtaccggcaaacccaagggaatcttgctcgaacaccagtcgttatgcaccagtagtgcctactatggtgctgcgatgggctatggcccaggctcgcgcgtcatccaattctcttcctacacctttgatgtcagcatcagcgatatcttcttctctttgatgagaggcggtgctgtttgcgtgcccactgaggatgagaagctcaacgacctggcggactttatcaacaggctcgaggtcaacaccgccgacttgactccatcggtcctcgaagcaacactatctcccgaggcagtcccgaccttgaagacgatatgtctcggaggtgaagcagtaaagcaagacaatctcaatgtatgggctgatcatgtggccctacacaattactacggtccctctgagtgctcagtcgcctgtgttggacgatccaatctctcttccaccgatctggctgccaacatcggagtcggatgtggtgcgctgacatgggtcgttgagagttccaacgcccacaagctcgtaccgcttggaactgtcggcgaacttgtgctcgaaggaccgctcctcgcacaaggttacttgaaactgcccgagaagactaccgaagtgttcttggagaatgttcaatggccaggcttcaaggaacccaaacgcctataccgaacgggcgatctcgtgaggtacaatccagacggaagcctcgactaccttggaaggaaggatacgcaagtcaagattagaggtcaacgtgttgagactggcgagatcgaacaccacgccgccctctgctgtcctccttcaatctctcaactcgcagtggaggcagtaacattgccagggagaagtggtttggcacttgccatgttcgtggcagtcggcgaccagccagatgcctgcatggaagttggcttccctcccgagctacactctgctctccttggcatgaaaaacagtttggcagcacatgtcccatcgtacatgcttccttcaatcttcgttccccttcgcgagattccccggagtctcgctgggaaacgcgaccgtagaactcttcaagagttcgtatcagcactcccggctgacatgattcgaacattctctctcgccgacgaggtcaccaccaagagagagccatcgactgcaattgagcgcagtctacgcggggtctggtccaaggtcctccgcgtgaaagagacggatattggtattgatgacagctttttcaaattgggcggtgactccatctccgccatgcgacttgctaccgctgcgaccaagagcggcctgcaagtttccgtgctggacatcctaacacacaagacaatttccaagattgccgcgctcgccgagaccaaggtcgatgaagcatacgccacccagagcgtcaccagctccgagcgcagtggtgaaaccttgggcaagtcgtctttcttggatgagatgcccgctcccttcgatcccaatgaggtcgaggtcatactgcccacaactagcgcccagaaagaggtgcttcaagcatgcaaggacagcccgaactctgactactaccacatttcccaaatcattgaggttcttccacaagaagtgggaggacgcaaggtagatgcggagctgctacacggagcctggcaacaggtcgtcgaccgccacccttcccttcgcaccgttctcatcaacaatgtaggacaagttatcctcaaacaacacatggccaaggtctcgcacattgaggtggaagaatcagacgacctgcaaaattacccaggccccgtaccactgtgggctgagttcagtccaatgcaccatgtcaccatcgcccagacaccctcgggtagaattctgtgtcgcattaacatacaccatgccatcactgatggcttttctctgatgaccatgttcgaggaattggccagcgcatactctggtacactcgactttggacccggaccgtcgctccaagagtacatctcccaggtcgagtccttggatcgcaaggcctcagcatcttactggaagaccgagatggacgcaatccaaccccggtccttcccacttaccaaatcaatcatcgcgactaacgacgccgcgcgcgctgatctacaagagctgcactgccagatcaatggcgaaaagctgctcgacttctgcctcgataacgagacgaccataccctcactagtgtacgcggcctggggtctcgtcctccaagccatgtgcacagatgatgtcaacggcggacaggacatcctgttcgcctatctctcgagcgcccgtgctatcatcccctcagaggccctgggattcctcgtcaacatcgtgccccaacgcgtccgcgccgcagacctagatgcggcatcactgccgcgcttcgtgcgcaacattcacgcgtccgtgctcgagtcgctacctcactcgcacattgcgccctctgagctcgggcttaaaatcaataccttggtaaacatgcgcaagttcgatgacgcggacatcctcgctgcattccatggcgatgaggctatggagcaactgagcagcgacggtcttgaacctgctgtcaagaaccaacaaccacaacagaaaccacatgaaatcacttttgatgcattcccttcgcccgaccccatggcagtaagttgttcccttccttgcttccttcctcttctttaatcccacctcggtcatgtatactaactttgtcttcctctacagtacgatgtcgtggtcgcggtctcggaactgggcggcgggacactggatgtcacgctctcgttctggaactcggtcgtggacgaaggcgacgcgcagcgcgctctagattgcttgaagagcgtgctgggcatgatgggacgggagggggagactatgacttgtgctgaagtcgtgggccagttggctgtctgatatgagacaacgaaaaaaaaaaaaaaaaaaaaaaaaaaacatgcacgatgacatttcctttgtagaatagggggaggtgttcaggatactgattgggattggttcttctttggtcgttacgtctcctacgttggtaaaggaagaaaggagttctggttcgtgggttatcttggttctacatctagttaatctctttcatctttcctttgaatattcatacactttaacaaccttc

SEQ ID NO: 2 - DNA, 25482 bp, unidentified
  source 1..25482: mol_type = other DNA
atgggtgtctccagcgatacaacgagccatctgctcgaagtgaagacgccagctatggacgctggggcgtctcccaagacgaggaggcgacgggccttgtcgggcgcggaggcccagcaagagttcggagcatccgccgctgcgaatctactgcagcaaatccaagcagcatggtccaaagccctaggactacctacggagctcatcggcacaacacagtcgtttgccagtctcggcggagactcggtggcagctatgaaagttgtctccatattgagtctagctggcatacgtgtgctggtgaaagatgtgatgcgagggaaatcaattcaggggctcgcatcagctgccaagcaggtggcagcaccagccacaaccgccgcctcgcaaccaccgccacaatcccaaccagaaccgcactcgttgaaggaagtcgcgacttgcgagacgatcaacaacaaactcaacaacttgaagcttgctgacgagcgccaagagcggctcgatcatctagcattcaaacatggatccgatactgaggtggaggatatgtacccctgctcgccgacgcagctgggtatcctcatcgcccaagccaagaggccaggtgcttatgtgatgcgcatcctgtttgaactttccatatgcaagggtaacgacaaagtcgatgtcagacgcctccagaaagcttggcaatctgttgtcgagcgtcatgcatcgctgcgcaccgtcttcgtcgaaggcaacacggccgaatccccgtttgagcaaatcgtcattcgacgcgtcaaggcggactttcgcgtcttgcgcatcatgaccgatcatgccagccaggaccgtcttgtatccctgggaaaccacggcggcgccctggccgagttcggtgaactgggtcatcgtcttaccgtcattgccaccccgtctggcaagcactacatccgcctggatatcagccatgctgtcaccgacggtggatcggtcgacatcatgattcgtgatctgaaactcgcgtatggtggaaagttggccaaaggccctggcccgacgtaccgatcgtttatcgacgaaatcctgcaccaaccctcagcccatggtctgcagtactggtcagagtaccttcgcggtgccgagacatgccagttccccactctcctcgacggagacatgtcctcatccccagaaagtgaactaatgtctgcagaagtcaatctttcatctctcacaaaagagattgcagacttctgccgcctccacgaggtgacccttttccacttgttccaggtttgttgggctgttgtcttgcgcgcatatctgaacaaagacgatgtctgtttcggatacacggcctcaggccgcgatgcaccaatcgactccattgagtccatcaccggagctcttgtgagcaccatgctctgccgtgtccaatttgatacgtccaaatcggttacaagcctcatcaaggaggtagcagagcgcaccacagaaagtctcgactttcaacatcattctcttgccgacgtacaaaaggccgctgatcttccaaaagacctcgttttcaatactgtaataggatttcagaccgctctatcgttggactcggatgacacccaagctggccttggggtcaaacagctcagcgtgtattcacctacagagtacgatatagctgtcatcgttgacacggcatccgaagccccttcggtgaatttggactattggacacataggatatcaccagcgcaagccgagatactcgccgatacacttgcccaagtaataacggccgtggtttctcatagcgactacaagattgggcaattggacgtcgtttcaccaaaccaataccagcaaatggttgactggaatatcgattcaactctggaacacggcaacttttctcaaggtacgtcaaccctacatggcatcttccgtgaacgagcgttattatgccctgcggcacccgccgtggtagcccacgacgggcaactgacttatagccaactcgacacactgtcaactcaactttctcatcatttggtatctcgtggtgtggaagtcgagacgctagtgccctactgtttttccaagtcgctatatacggtggtggctattttgggaatactgaaagcagggggtgctgcggttccactagatcccaatcaccccccagatcgcctcgacaccatccttgacgatacgaaggctgccgttattgtcacctccccagaacacagtcatctgttccagcacagacccgccgcaattgtttccgttgatgacgcctttcttcgtctgctcccagaggtctgggaagcccctaacgctgccccgacggccgactcggcagcattggttgtgtacacatcgggtagcactggtgtccccaagggagtcgtgtcgattcatcgagcagtggctacgaattgccaagcttatggcaccgccctccagatgaatgagaagtcccgcgtgcttcagtttgcctctcacactttcgatgctagcatattcgatatgtttgccgcccttcttctcgggggttgtgtgtgcattccgtccgaggaggataggatgaacgatttgcctggatttatcgagcgcacaagctgcaactggattcttgctacgtcaacggtcatgagcatgattacaccgcaacaggcaccaggtctcgaggtccttgtgctgggaggagagccactctcccggacagtgcttgagatgtgggcaccttttgctaccgtattcaacgcgtacggcccggccgagagtgccatcatgtctacgtgctcaccgaagctcactgctgactcccaaccatctctgatcggaaaagctgtgacagggcctgtgtggatcacagacacgtcagatcacgataggctgatgcccatcggatgcattggggagatcagcgtccaaggtccaggagtgtcgcgaggctacctgaacgcgcccgacaaaacctcaaaggtgtttttcgacagtcccgtatggtcgcatccttcgtttgagccgtattctcgattatacagaactggcgatctgggccgcctcaacagcgatggcttcgttgaaattctgggtcgaaaggacaaccaagtcaagttgcggggtcagcggctagagttgggcgaggtggagtaccagatcgcatcgcacgatcaggtcaagcagtgcactgtcgtgaagggcggaaagggtctctgcaaggagcgtcttgtgggtcttatctctctccacggagagccaaagtccatggacactcaaatacgatttgcggaggacaacgacaatgccaaagcccaggtagtctccgaaatccgagaaaagctggctggtgcgctgcccacttacatgatccctacgctttgggctgtcctgcaagagctgccaatcaccacctcgggcaagatcttcaccaagggcctccaacaatgggttgatgagatgagccaggaggccttcgtccgagtcactcgatcatccaaggacggcgagagcgaaaaggatgaagaatcgaggacgaagatgtcggggactgaacaaaagatccagaagatctgggcccgtgttctcaaccttcccatcgaagcgatcacgcggtcaaacacgttcttgtcagtaggtggagattcggtaactgcaatgcgtgttatgtcactctgcagagccgaaagactcctggtcatagttcaggaggtattgcgcgaccagacgctcgctagcttggcaacttgtgctaagactatgggttcgaccaccgcatctgacgacgtcgaagatatatatgacacgccggttggattgagcccaatccagaacctcttcttcgcgacacagggtgtctcgaaccgatggaaccagagcttcatgctcgagttacgaagacctattccactggagagccttcgtggcgcattgaattctctcgtcaagcgacactccatgttgcgtgcccgctttcaaaagacatcgacagggacatggacgcaacagatcccccgaccagatgacggtgtagttcatcagtgcttcaccttcgacactactgatatatcaggagatgtctcaatggtagagcatgcgaacctggtccagtctcgactcaacattgaagatggacgtgttttcacagcaacactgatgagcaagggtgaagagcagtacctttacatggttgcccatcatctggtcattgacctcgtttcctggcggatcatcatccaggacctcgaagatatgctatcgaacaccgcgctcagcaatactctggcgacgcccttccttcagcttactaaagagcgtattgccaagttgggcgacgcaactgcacaccaagatcagagtcgaaatgtcaactctgcggcaaactggagctactgggattttgccgaacagcccaacatcgaagaggatgcccgtaagctgagcttttcgctcgaccaagatgtgtctgggcagcttcttggcgcttgcaacaatgtacttcggacagaacctaccgatctgtttgtcgcggcccttctcaattcgttcgcagaggtcttcacagatcgcaacgtgcctgatgtattcgtagaaggccacggtcgagaccatgaagcgttccaggaaatggatgcgaccggtactgtgggctggttcacagccatttggcctctaaccattccgattacatgggcggggcagcatgtggatctcacgcagacgattcgagccaccaaagacacaagaaggggtcaagagggccctggcaagaccttcctggattctgtcatagccccaggctcatcggatggcgtgccaatggaagtcatctttaactacgaaggacaatatcaacaattggagagttcggatggcctattccggatgctacctctcccagcccctgatatggcttctgatgtgcagcgcactgccttgtttgagatcaccaccgtggttaagggaggccaggtgatgtttgaattcttattcaacgccaagatgggccgtcaagaccagattggcgagtggatcaagaagactgaacttaccttaaccaagcttgccaccgagctgcccaccagatcaaagatgtatagcttgtccgacttccctctcctccaacttgattacgacaatctgcaaacttttacccaggggctagaagttcgacttcaacctttgcgccaacgggatagctcgtttgaaattgaagaggcctacccctgtacaggtatacaggaaggtctactcctatcgcaagggaagcagtcgggaaactatgcagtgcacgagacactggaggttgtttcagcttcaggaacacctattgatgcctcgcgtctaagagacgcttggctccaagtcgtcagtcgacaccctgtgttgcgaaccgtctttgtggaccacccgacggatacggggtatcatgtccaagtggttcttcctcgtgtgatcccggaaatcaacatttcaacgtcccaatcggtcgacgaagctctgttgctgtggaagaacctggggaagaacgattatcccgctgggcaactcatgcaccgctttagcatctgcagcactgctgggggtcgggctctggtcagattcgacttcagccactgtatccttgatgcccagtcttgccaggtgctattcaaggatttgagtcaggcctacgctggaacgctcgacatggacggaagtgtgccacgattcagtcaagttctgcagcatatgcaggggcgtgatcaggcttccgacctgacattttggcaggctgaacttactggcgcggaaccaagcctccttccgaatctttccgactcatctgacgagaccgagcttcatatccagcccctcgaacttggtatatctgccgacgccgtgcacaccttctgcaagcagcacagcgtgagcccgtttgcgatcttgcaggtggcctgggcccgggttctcagcgcctacacggcgatggacaaggtgtcctttgggtacctcagctccggccgcaatgccccaatcaatgggattgaggatatcgccggggttctcgtcaacatgcttgtcacgcatgcgaaggtcgactatagtgtagccgcaattgatgcggcgaaggacatgcatcgcagctacaccgaaagcctggaccaccagtactgctcgctgacagaagtccagcatgctcttgagctcggcggacagccgttgttcaacactgttctcgacttccaacgacctcccgaggaagatgatttggacacagctatctccttacgtcctctggactttcacgatcctacagagttcgctctcacactacactttgagctcaatcataaatctttctctggctacatgtctcactgggcctcgacattaagccaatcatatgcccagagtattggagaggccttggtactggccctacagactatcatggcaaagcccgacatcaaactcggtagcattgacctcatcagcaacaagactctggacaagtacatcgacatcaacactccggcgcccgttgccaagtctgagtgtcttcatgacaccttcagacgccgtgtacaaatggtcccagatgccccagccgtcttctccactgaaatgagtctcacatatgctgagctggatctcctatcaaccaagctggcaaatgagctcttgggcatggacgtgggtcccgagacgaacattctgctgtgctttgagaaatctgcgtggacgattgtgtccatgatggcagtatggaaagctggcggctgtgttgtcgcacttgaccctacacaccctgcttcccgcctaaagggcatagctgaagacgtggctgctcgcataatcctggtctcgccagctactattgacacttgtcgcgaaagcctcgacgcgcagatcagattcatcgttgtcgactcaatgaacttgagcgcaatgattgatgagcggcccaatgtcacgcccgtgaagccggagaatgctgcttacatcaatttcacaagtggcacaaccggcaaaccgaagggtgtggtcattgagcaccaggccattgcaagtaacgtagagcccttgactaaagcggcagacatcaacaacacgacacgagtcctgcagttctcatcgtatgcttgggatgctttctactgcgagaccatcatgccccttctgtctggtggatgtatctgcgttgccaacgaccacgaaaggtcctacgatatcgccgagttcatccgacgcgctaactgcacctgggcgctgtttacaccatcattcgcaaggctcttgacgccttctgacgtgccaaacctccgcaccctactcttgggcggtgaagccatgtcagctgatgatgtgaccacttggtctgagtccgtggtcctcaagaacgcgtatggcccatgcgagtgttctatcgtcgctcttatgcaccccaggattgacgcagcccgcagtgggtcgaacctgggacatcgcttgggccagaccctttgggttgtcgaccctagaaacccacagagacttgcgccagtaggttctccaggtgaactgctcctgggtggtcctcctgtggcgcgagggtatatcaatgacccagcaaagactgccgcggctttcatctcagctcctgcatggttcacatctaacgctgtcctgggcgagcaaggcttcacctctcggttctactgcacgggggatcttgttcgtctcaatgcagatggaaccctcaacttcctcggacggaaggatggagaccaagtgaagctgcgcggccagcgcatggaactcggcgaggttgagcaatgcatcatgacaagtacgtcgacattgaagcaagtagccgccgcggtgatctcccgcaccggccatgccggggacagacagcttgttgccttcgtcaacacgtcggactctaccgacttgaacagcaagccacagacattgcccatgtcggatgagttgctgaagagcctccaggcgctgacgacaaccatggcgaccaaggtaccctctttcatgataccttccatgttcatcccagttaatcgcataccactgagtgcatcgaccaagatcgaccgaaaggctttgaccgagctggcttgccagatccacgaatccagtgtcgctcgttatgcactgactggactttccgaggcttgccccccgacaacgcccatggagataagtcttcacaccctctttgctacagcattgggcgtcgagaccacgtcgctttccatcgccgacaacttcttccaccgtggaggcgactctatcaaggcgatcaagctggtggcggctgcgcgacttgcgaatgtcaatatgacagttgtcgacgtcttcaatcacccaactgttagagagcttgctgctgtggccaacttcatcaactccacaccagacgacccattacgaaatgatagtctcgaagtcttcgtgaaggagatgaagaccgaggtcaccaatgtcgcaattgaagcacacaagttgaaagcgggtcagattcaagatgtgtatccctgcaccgagctgcaggacgcgatgttgtcgataacactgggccagccaggcgcctatgtcctgcaacttgtgacctcaatctccgaagatgcggatcttgaagcctacattgacgcttggaaggccgtgagaaggaataatgacatcttgcggaccagaattgtgagcgcatttgggaagcgctaccaggtggtgtacaatgaccgcatgataatcaagaatgaggagtctcttgacgattatctcgcttctgacaagcagaagcctatggcatatggcgacgcacttgctcgcttcgccctgatctccgaaggagaaaagcattacctagtgtgcaccatgcatcactcaatctacgatggatggtccgtttcactcatcatggatcagctctccgaggcctatcagtggaagccccttcagaagccacagccattctctgaatacgcttggcacgttttcaaccaagatcgcgttgccgcggaacgattctggaccgctgaattggccggcgccacgacatgcgacttccctcgcatgccatcacctacctaccaagccaacgcccgtcagaacgcagcccgaactatggtccttcaacacagccagtcagacatccccctcgcagcaatcatcaaggcagcctgggcgcttctattgggtcaacactccggctcgcgtgatgtcgtctttgcgacaactaacgctggtcgcaacttggatctaccaggcattgctgacatgatcggcccaactattgccacagtacctgtgcgtgttcgctacgattcatcagatagcatcaaagacatggtgaccaaggtgcaccaggggtctctttccatggttccatttgagtatcttgggctgcagcacatccgacatctggacgcctcttgcgaagccgcttgccaattgcgaagcttgctggtcgtccagccaggtgctctcgacgatgctgacattctgcccatccctggtgtcgagaatgtaccagtcgacacaggcaacttctttacgcagcccttggtggtggagtgtaccttgggaaccaacaaagtgaagctctcggctacctacgatgatcatgtcttgagctccgcagagacggatcgcttgcttgctcagcttgagcacttcatccggcaattgagcatgaatatgacaaaaactgtgggcgatatggaccttgtctgccccgaggacaggctcgacctcaactcttggaacgccaatgagccgcccaaggtgaactcgtgcgtctgggatgtgattgcgaagaagacacaagaaacaccaactgccctcgccatctcatcttgggacggtgaaatgacttatgatgaattagacaggcattcgacacgccttgctgctcatctctgtcattcgcttggggtcaaatgtgagacactcgtcccgttggctttcgagaagtcggcatgggctatagtggcgatgctagcggtcgtgaaatccggaggtgcgttcgtatttcttgacgacaaattgccaccagcacgacagcgattcgtggtgacatcaacccagtcccgattcgtcctcagctcgtccaagaatctccccggctggaaccactctcaatggcggagcgtcgaggtcagcgaagcagccataggacagctgcctgaccaagttgcgcccacgcccagctaccagccggacagcatgttatatgccatatatacatcaggtagtactggtaccccaaagggctgtgttgtggagcacgccgcattcctgagcagtgccgctagctacaccaagctcctgaagatgggtccagacacccggtcgcttctgttctcctccttcgcctttgacgtcagcctgatggagtctcttgcggctctcacagtgggtggctgcatctgcatcgccagtgaacaggcatatgaggcaggtgtcgccagcatgattcaggaagccaagcctaactgggcatctctcaccccatcagtggcaagattgatggagcccagagatgtgccttctctcaagacccttgccctcgtaggggagcctttgagccaggaagatgtcaagacatgggctggaagcctccagcttatgaatggttatggcccgacagaatgcgctgttctctgcctcattaatgccaacatgacgacagagaccaaggctacaaataccggctatggcagtggtgggcttacttgggtcgtcgatcccaacgaccctaacattctcatgccaattggctgccccggtgagctcctcatgcaaggtccgatcctagcccgcggctaccttgaccttcctgaggtgacagcatccgcattcattaccggcctcaagtggtcgccccaggatcggttctacaagactggtgacattgtgcgatacagccccgacggatccatccagtgtttcggccgcaaggaccaacagattaaagtccgtggacagagagtcgaactgggtgagattgaggagaacatcgatacactcgactctgtcagccaggcatgtgtggtgttcccaaagtcagggccatacgagaagaagatcgccgctgtcgttgtgctcgaggagttcgcagctcttgactcagctcctctccacctcatttcagccaagcaacaaaaattcgcaggcatcttggtcaaccaaatgcgtgatgccctatcagagaacctgcctagctacatggtcccagaagcctggatcctcgttaccgctctccccatctcaatttcgggtaagagagaccgctcaaccatcggccgatgggtaacggaaatcgacacaaagcaacaagagcagattgacaacctcctcaccccaaccacgattgagtttgccaagacagacgacgagaagaggctacattccattgtgactgaggtgctcaatattgacccttcgcgggcgggtatcaaccagtcattcatgagcttgggtggcgactctatcagcgcgatgcggttaatagccaagtgtaggagccaaggtatcacactcagcctcaagaagttgatgagcagagccagtctttcgcagttggctacttccatgactgcgaatggtagccaaagtgctacagctcaagtcgcctcggacgacaccgggaaaccattcgacctcacaccaattcagcaacactatgtggatcttcacgctcaatgcccgcctctggatacggaaacagagtttaccggccggcgtcgattccaccaaagcttcttcatgaagctcacaaacacagtcagcgcttcgaatgtgactgtcgcggttcgaggacttgttgagcggcacccaatgctgcaagccacattcggacaagattcgaagggtcggtggcaacagactgtaggcagggctcccagtacagttgattgcatctgggtcagcgacgcccaaaccatgcaagcagcgtcagcaacactcgcttccgccaacagcctcgacgtgatcaacggtcccgtattccgtgcagtcttcttttcgaatgatgttgacggtcaaggacaattgcttttcctgacagcgcaccatctggtgattgacttggtgtcatggagaatcgtgctagaggagctagaacagagccttcgtggccagccactacctgagccaactggattgagcttccaatcatggatgaagctccaagccgaccatgcccggctcaacctcagcccaaaatcggccttgccatttgacgtgcatgccagtaacctctcttactggggtatctcgacaccgtctgagaatacttacggcatgaccaagaccgaaagcttcaacttgagcaaggatcttacctctctactgatcggccaatgtcatgaagtacttcgcaccgatgttgtggatgtactcttggcgtccgcgttccgggctttcaccgacatcttcactgagcgagatggtcccacagtgtttgtcgaaggacatggccgggagccttgggatgagtctatcgacattggttcgactgtcgggtggttcactgccctgtgccccgtccacgtacccaagaaacccgaagacacaccccttgagaccctgcgagaggtgaaggatgtgcgccgcagcattccccagaacggatgggcgtacttcgcgtcgaggtacttgaatgctgatggccgagctgccttccaagacgacttgatggcggaaatcatcttcaactacgctggacaatttcaacaactggaggctgacaaccagttgctcgtccctttcgatggcgaactgcctgaagaagcatctgatgttggccctgacacgccgcgatttgctttgatcgaagtgtccgctgtcattcggaatggtcaattgcagatgtcgttcatcttgaacgatcgcatgaagcacgtcgaccgccttggagaatgggtgcgctcggttcagaagaccctcgagactcttatcagcagtctttgcgaccaaccgcagaaatcggtaccgacgctgagcgacttcccactcctacgtaccacccatggcaggcttgatgatcagatatctgaagcatgcgcccggttcggtgcttcggattggactgaaatggaggatatgttcccttgttcaccacttcagaccgcaatgttactgaacaactctatgaatgccgaacaataccgctcatgcgtgatcttcaacataacttcccctacaccaatcaccgccgaggctctgagcgctggttggagcaaggtcgttgcccaccacccttcgctccgaactgtgttcatggaaaccaagacagataatggggcatttgatcaggttgtcttcaagaacctacaacctcaaatcaacgtcctagatatacagggcgatcttgccctcgctactttgaaagcccgcggcccggcggattggtcaaatggcgctcctcatgtcttgaccgtcattccgacatcagctggcgtgctatgcaagcttgagattagtcatgccctgatggatggcgcgtcaatggatctgctatttggcgacctcgtttcggctttggaagacaatctggatgctgccccgagcccttcttaccacggctacattgcgtatctccagaatcgagatgccagtgattccttgacacattggatgagctacctcaaaggcgctaacccttgtctcatctctcctatcaagtctgttacgcagcctgtcgagatcaacgaggtaacttcaatcgaggtaccaatagttgatggcaccgcacaagccctcaacaactttgtcgacgcccatggaattacacaggcgtcggttatccaggctgcctgggcgatggttctgcgtcgctacacccagtcagaagatgtttcgtttggttacgcagttgcaggccgcaatttgccaattgaaggcatcgagaagaccgttggccccttcatcaacgtccttccgtgcagacttgacatgagcgacgacaagctggatatccgtggcttggccaacaaggtccagcaagacttcttcgagagtctccctttccagcatacctcgcttgcggagatgcagcatgagttgcaacttccgacccgtgcgttgttcgacacggtggtgtcaattcagcgttacaatgaggcgtccatgatctcttcaaacggccttcaaatgaatcttctgcacagccgcgatccaacagaatttactctgactctcaacgctgcgtactcgactgacagcttgggctttgttgtgagatactctccgcaaaacggtgttacaagctctagggcggaaagcatcgcgcacacgttccaagctgccctgagtgcgatccttgccaatccagagcagacacttcggaacgttgacctggtcagcaagcatgaccgtgacattattgaccaatggaatgcccatgtacctgaagcagtcgatcaatgcgtgcaagcaagcattaagctgcgtgcagtttcccaacctgcagctgcagcggtatctgcatgggatggagagctctcttaccaagagctggatgatcagacaaaccgtctcgcgggacacctagtacgcaagggtgtcaaagccggcaccattctgccgttctgcttcgagaaatcgatttggaccactgtggcagttctgtctatcttgaagacaggcgcttcgtttctccctttgaacatcaacgaccccgaagagcgtgtcaataccctcctccaggaggctaatgcacaccttgttctcgcgtcgcctagccaatctcggaagtttgaaggagttgtgaatactctggtcatctctgaatcattcattgcggctctcccaccaactagcgataatgagttgcccacaattgccccgagtagcgttgcctatatcctattcacgtcaggttcaactggcaagcctaagggtgttgttattgagcaccggtcgcttgctagtaacatcgcagagcatgcgcctggtctcggactcaattgctccacgcgtgcgctccaattcgcgtcatactctttcgatgcatcgattacagaaactttggcgacgttggtgtcgggaggctgtgtctgtgtcccgtcggaggaggaaaagacggccgacatcgaaggcgcaatgcgacgcatgcgagtcaacttctcgatgctcacgccttcagtggtacaactgttgtcgcctgagaacctcccatctttggaaacactggcgcttgttggagaagctgtaccaaagagcttgatcgagaagtggtccaagaaactcaatcttcttgttggctacgggccgaccgagacggcagtttttgcgtcgattggaagtctacagggccccaacgacgctgggctgattggcaaagctattggcactcaaacgtgggtggtcgaccccgctaaccatgacttactggcgcctatcggttcactgggcgaactcgttctcagtggcccgacgctagccagggggtacctaaacagtcccgacaagacggaacaggccttcaagacaaccccggcctgggctacccctggtataatcaatcgcgtttacaagactggtgatttggtatactatcgcgacgatggcaatctcatgtacgccggccgtattgacggtcaagtcaagatccatggtcaacgtatggaagtcggcgaagtcgagcaccagatccttgagtgccttccagcgcattcggagacagctgttgctgtgctgaaatccgacaaaacctcaattcagcgcttgatggccttcataagcatcccccagatcgaggctgcgcctgcaccctctccgcgactcgccatcttctctgacaaattaagagatgtgcttttgaagttgcagaaggatctgtccgagaaggtcgccacttacatggtcccctctttgtatattcccatgtcatctctgcccaaatcagcagctggcaaggccgaccgtaaagctatgttgctgctcgccgggtcactatcccaggatcagatcgatctgtactccctacgagcctctggtggacaggaagtttcgaccaaggcggagaaatttgtgcagtccgtctgggcatccatgctggggattccggctgataccatatcatcggatgaccatttcttcaaactaggaggcgactccttatctgccatgaaggttgcatctcaaatgcgcagcgagaatgtctttatctccgtcactgagctcttttcgcatccagttttgtcggcgctggccacactcgtggagtcaaagacgggagcggcggaggtcgtatcggtaagcgacgtatcgcccttcgagcttctgtcagacaagcaatgcgtcatcgacaggatcgagaagaccgtgggagtatccatgcatactgtcgaggatgcgtacccagcaacgcctctccaggaagcgatgttcgcaatcacacaaacgagcgattccgcttacgtcaatcgcatggtcttccagattccttcgcacatcgacttgatccgattcaagaaggcctgggaagccgtgtatgctgtcacgccaattctcagaacagcgctcgtcaataccgaggagcatggtacttgccaggttgtatacaaccgtcctgtgccttggtttgaggctgaatgcaacctcgcaagctacttgtacacggattctcaggggcccatctcggaaggcgatgtcctcaccagatacgcaatcatccatgatggtcccgcacattggttcgtctggacagctcaccatgcggcctacgatggctggtcagctggtgcacttttcgatttggtccacgcagcttatgagagtggtgtcgctgttcgaacaccatcttacagcaacttcatccgatacgtctgcaactcgaacgtgaatgatgtcgatgaatactggagcaccacacttgatggttatacccgaacggatttccctccaattccacacccagattaccgagccagcagtgaccaatcagtcgttcactccattcaactacccgccaccagacagaacgccgatatcacccaagcaaccatcgttcgagccgcttgggctttggtggttggcaagcagagtggaactactgatgtcgtcttcggtatgacccagaatggacggaacacacctgtcgcaggcattgaagagatgcaaggaccaaccatcgcaacagtccccgcacgtgttcgaattgacatctcaaagagcatatcaacatacctgcaagagatgcagcagcaagcagccggctcgatcccttatgagcaagccggactccagaacatccgcactttgaaccaaggagcagaagaggcctgcgacttcaacaacctgctcgtcattcaacccaagggcgaagcgggcggagaagtctggctagagcctgttactggagaactgaacaaattcgacaatttcccactgactattgagtgtgcgattggaaaggagggacatatggagctcttggcccacctcgactcccatctgctctccaaactccaggttgagcgcatcctcttccaatttagccatgtgatccgtcagctttctgaggccaacactgagactctactctctgatgttgagttcttcactcctgaggatcgtgctaccatcttcggtgaatggaactccaacatcccaggcactgttgaagcatctctacaagacagattcgaagagaatgcccgcgaatatccacagaagcaagcaatttactcttgggatgcgttaatgacatattcggagctcgatcagatgtcaacacatcttgcacacaagcttatcgatgcgggtgtgaaacctggggagttcgttccactctgtttcgagaagtcagcgtgggccgttgtttctatgctggcggcgatgaaggctggagcagcctttgtgtcaattgatcccgagcatccagagaatcgtcgtcaggatataatcgaccagaccaatgcccaagtcatgctagcttccgggacagccccacctctatcggtcgaccagaccttggtcgtctcggccgatacactatccggctatcgacaaactaaggactcatctacattgccccccaaagaccctagcaggcttgcctaccttatctttaccagcggtagcactggccgcagcaagggggttatgatgtcacaccgagctgtctgctctagtgctcagatgttagacttggaaggtcttggtttcgcccgggactccagagtgtaccagttcccctcttacgtcttcgacgcatctatcttcaacatcttcgggactctgtctgcaggagccacgctggtggtaccagctgacgaagacaggcttggtgccaacctagccaagtcaatgcgtgacgcggaagtcaccaaagccatcataaccccgaccgtcagtcggttactccaaccagaggatgtaccaactttgcgcaccctcaaactgggaggcgatgctgtataccaagaggatgtcgacaagtgggcttcgaaagtacacatgctgcagggttacggcccagcagaagccactgtctttgtcacctggacagagataactccccaaagcgtacctaacaatgtcggacctgccagcagatccaggctttgggtggtggaccccgacaaccacgacaagctgacacccataggcatggttggcgaaatgctcattgagggccccaccctcgccaacgggtacctgaatgacgaagagaaaaccgctgcggcattcattacaagcccggcatggtgtcataatgagtcaagacgattttacaagacgggagacctagtccgctatgccgataatggttcgttcttgttcatcggtcgcaaagatgcccaagtcaaaatcaatggccagagagtcgagctcggtgagatcgagaaccatctcttcaacatgttgccatcatcccaattcgtcgctctcgttccaaaagccggtccctgcaagaacgttctcactgtactagtggccgcgacaaccgctgactcagtcaagagacttgagtttatcgaagatgcctccacgaaggagtctttgagcaaggctgtctccgaactgcgtgaagccatgccttcccgtgtccccaagtacatggtaccacgtacatggttcgcggtcagcaggctacctcttacttcgtcgggcaaagtcgaccgcaagaatatccagcgctggctggaagagcttgaccccgacgtcgcacggatggaaacactttctcttaagcacgatgccgccgctacaatacccacgaccaagacggagagtgttgtacagcaggctattggccgagtcctcaatctcgcacctgagaacatcccgctcgacaagaccttcatggccttgggaggtgactcgatcactggaatgcaactggtatcccgacttcaatcgtcaagcatcgtgctcagcgtgcgacagctgctccaaagccataccatccgacaccttgcgtcgcttgcacaacccgaaggcgaacgggtggagctagcggatgaagtggttggcgtgcctttcgatttatcgccaattcagaccatgctgattgacaccatcttgcccgatgcaatggcacctcggcgtttcaaccaaagcttccttgtccgcttgagtcacgaggtggatacagggaaattgcgagatgccttcacttaccttgtatctcgccattccatgttgaggtcacgttttgagaagacctcagatggccgctggagccagagaataatgccagagtcagacagcgcataccagctctcaatccatggaacgtctcatgatctgggcaagatcatggcggatagccagagcggcttagatgtcttccaaggaccagtttttgcaggtgatgtgtgcgccgacaatgaaggcaagcaagtcttgttcatggtcgcacatcatcttgccgttgatttggtatcatggcgtatcatccttaccgacctcgaggagtacctgacaacagacaacttcaaggagaaacacaagccgttgtcgttccagacatggctatcactgcaacaaaagaaggcggaggatattcgaccggacactgctcttccctttaccccaccacaagcgcctatcaactactggggggcactcactgaatccaacacctttagcgccgtggaggaacagtcgttcacactcggtgcggatctaacatcgcacctactcaaggactgcagccaagtgctaggaactgacacggttgatatcctcatcggcgccttgacccatgcattctcgaaaacgttccgggatcgcgacctgccgactgtcttcaccgaataccacggtcgcgaatcttgggatgattcaattgatatctcatctaccgtgggttggttcaccaccctggcgcctgtgcatgtcactagccatggcgacgatggtatcaaacctttcgtccggaaagtgaaggattgtcggcgaaagaccccaatgaacgggtggtcttactttgcttcgcgcttcttgagccccgaaggcagagagcgattcgcccagagcggacccatggaaattgtgctgaacttcgtcggacagtaccagcaactcgaacgagccgaaggactcttcgagatggtccctcgcgatgactttggctcggacaccgtcgacgtcagcccagatctggaacgctcctcaatcttcgagatttcgtctggcgtgatgaatggccatctgaatttctcaatgatgtggccacgaagtcttcgcaaccaaagccagattcgacaatggctgaccaataccgaacaatctctgatcgaagctatcaaagaccttcaaggtctcactagagagttgactgtcagcgacttccctctgctctctatggcagatgacgagttcgttagctttgtggaggactctttgccaaagcaccacatcgctgttggggacgttgaagatatttaccctgcgacaccactgcaagaagccttaatgatgtcacgaagtatcgactccgaattatacaccgtacacgccatctaccagctcagctgcaaggatggcgagcctgttgatacggagagactcaagaccgcctggaactccgtggtcagccgtcatcagagtctaagaactgtcttcgttggcgttgtgtccgaccacagtgcactcaatcaagtcgtactgaaggcgatgcctaatacgacagtggaattgacttgcgatactgacgaccaggccctggaaatgctcgaaaagccaaacacactgagcccaagtctagcctgggaggtagaaaccccagcgcagctgatcatctgccatgctatagcatccggcacaacttactgcaagatcgccatcaaccatgcggtcgccgacggtggctccaatggtatcctcatccgggacatcggcctggcctacgaaggcctcctctctgcagagcgtggtccgctatacagtcaatatgtctcttacctccagagccgcgacagacaagcaagcctcgactactggggcgaatatctgcacggtcttgaaccgtgcatggtcaatggtggcacacgctctgggaagaccaaggaactccgtgatcttgaagtcgagatcccacatcccgacaaattgagagctttctctgctgcgaggtctgtcacactcgccaacgtcttccaagcagcatgggccttagtactgcgcaacttcgccggcacagaccaagtctgctatggttacctcaacgccggtcgcgattcacccgtcgacgggattcgcgatgcggtcggactttttgtgaatatgctcgtgagccgtgtcgatttcaccgagaaccttacagcaggagagctagttaagcaggttcacaatgactacatcgacgccctcgatcaccagaactgttcactggccgacatgcaacggatcgccggccatccgctattcaacagcatcatgtcgtaccaggtcgcttcatcttcgtccgaggagacctctagcgatctcaactttgcccaagtcagcgctcatgaccccactgagtaccctcttagtatcagtgtcgcggtttccgacaagtcgatcttcgtcacccttagctactacacagatctggtttcagattttcaggcgcagagtgtggccaccagcttcgagaaggcgctcggcgggttcactgaagcaacactcgaccttcccgcgcgcaacattccaattctcggcgataaccacatctccaagatccaagagtggaacgctgaggggcccaaagcggttgaaggatgcattcaccagatattcgaggaccaagtcaaggcgcaccctgacgccatggcggtgtgttcaaccgagatcagctggacctacagccagttggatgtgctttccactcgcctggcacatcgcctcgccatcctgggtattggacccggcaccatagtcccgtactgcttcgctgcctccccatggacggtggtgatcatgatggctattctcaaatcgggaggtgcctgcgtggctctggatcccaaacatcccatcgatcgtctgcacggcatcatcgaagacgttgacgccgatattgtcattacttcgtctcgccacgcttccaagttcttaccggtggcggcggtattggcgctcggtccggaagaaattactcgcattggcaaaaccgcacccagacggcttggcacgttgccaatcgctcaaccaagtgacgtcgcagttgttcagttcactagtggcagtaccggcaaacccaagggaatcttgctcgaacaccagtcgttatgcaccagtagtgcctactatggtgctgcgatgggctatggcccaggctcgcgcgtcatccaattctcttcctacacctttgatgtcagcatcagcgatatcttcttctctttgatgagaggcggtgctgtttgcgtgcccactgaggatgagaagctcaacgacctggcggactttatcaacaggctcgaggtcaacaccgccgacttgactccatcggtcctcgaagcaacactatctcccgaggcagtcccgaccttgaagacgatatgtctcggaggtgaagcagtaaagcaagacaatctcaatgtatgggctgatcatgtggccctacacaattactacggtccctctgagtgctcagtcgcctgtgttggacgatccaatctctcttccaccgatctggctgccaacatcggagtcggatgtggtgcgctgacatgggtcgttgagagttccaacgcccacaagctcgtaccgcttggaactgtcggcgaacttgtgctcgaaggaccgctcctcgcacaaggttacttgaaactgcccgagaagactaccgaagtgttcttggagaatgttcaatggccaggcttcaaggaacccaaacgcctataccgaacgggcgatctcgtgaggtacaatccagacggaagcctcgactaccttggaaggaaggatacgcaagtcaagattagaggtcaacgtgttgagactggcgagatcgaacaccacgccgccctctgctgtcctccttcaatctctcaactcgcagtggaggcagtaacattgccagggagaagtggtttggcacttgccatgttcgtggcagtcggcgaccagccagatgcctgcatggaagttggcttccctcccgagctacactctgctctccttggcatgaaaaacagtttggcagcacatgtcccatcgtacatgcttccttcaatcttcgttccccttcgcgagattccccggagtctcgctgggaaacgcgaccgtagaactcttcaagagttcgtatcagcactcccggctgacatgattcgaacattctctctcgccgacgaggtcaccaccaagagagagccatcgactgcaattgagcgcagtctacgcggggtctggtccaaggtcctccgcgtgaaagagacggatattggtattgatgacagctttttcaaattgggcggtgactccatctccgccatgcgacttgctaccgctgcgaccaagagcggcctgcaagtttccgtgctggacatcctaacacacaagacaatttccaagattgccgcgctcgccgagaccaaggtcgatgaagcatacgccacccagagcgtcaccagctccgagcgcagtggtgaaaccttgggcaagtcgtctttcttggatgagatgcccgctcccttcgatcccaatgaggtcgaggtcatactgcccacaactagcgcccagaaagaggtgcttcaagcatgcaaggacagcccgaactctgactactaccacatttcccaaatcattgaggttcttccacaagaagtgggaggacgcaaggtagatgcggagctgctacacggagcctggcaacaggtcgtcgaccgccacccttcccttcgcaccgttctcatcaacaatgtaggacaagttatcctcaaacaacacatggccaaggtctcgcacattgaggtggaagaatcagacgacctgcaaaattacccaggccccgtaccactgtgggctgagttcagtccaatgcaccatgtcaccatcgcccagacaccctcgggtagaattctgtgtcgcattaacatacaccatgccatcactgatggcttttctctgatgaccatgttcgaggaattggccagcgcatactctggtacactcgactttggacccggaccgtcgctccaagagtacatctcccaggtcgagtccttggatcgcaaggcctcagcatcttactggaagaccgagatggacgcaatccaaccccggtccttcccacttaccaaatcaatcatcgcgactaacgacgccgcgcgcgctgatctacaagagctgcactgccagatcaatggcgaaaagctgctcgacttctgcctcgataacgagacgaccataccctcactagtgtacgcggcctggggtctcgtcctccaagccatgtgcacagatgatgtcaacggcggacaggacatcctgttcgcctatctctcgagcgcccgtgctatcatcccctcagaggccctgggattcctcgtcaacatcgtgccccaacgcgtccgcgccgcagacctagatgcggcatcactgccgcgcttcgtgcgcaacattcacgcgtccgtgctcgagtcgctacctcactcgcacattgcgccctctgagctcgggcttaaaatcaataccttggtaaacatgcgcaagttcgatgacgcggacatcctcgctgcattccatggcgatgaggctatggagcaactgagcagcgacggtcttgaacctgctgtcaagaaccaacaaccacaacagaaaccacatgaaatcacttttgatgcattcccttcgcccgaccccatggcatacgatgtcgtggtcgcggtctcggaactgggcggcgggacactggatgtcacgctctcgttctggaactcggtcgtggacgaaggcgacgcgcagcgcgctctagattgcttgaagagcgtgctgggcatgatgggacgggagggggagactatgacttgtgctgaagtcgtgggccagttggctgtctga

SEQ ID NO: 3 - RNA, 25717 nt, unidentified
  source 1..25717: mol_type = mRNA
atgggtgtctccagcgatacaacgagccatctgctcgaagtgaagacgccagctatggacgctggggcgtctcccaagacgaggaggcgacgggccttgtcgggcgcggaggcccagcaagagttcggagcatccgccgctgcgaatctactgcagcaaatccaagcagcatggtccaaagccctaggactacctacggagctcatcggcacaacacagtcgtttgccagtctcggcggagactcggtggcagctatgaaagttgtctccatattgagtctagctggcatacgtgtgctggtgaaagatgtgatgcgagggaaatcaattcaggggctcgcatcagctgccaagcaggtggcagcaccagccacaaccgccgcctcgcaaccaccgccacaatcccaaccagaaccgcactcgttgaaggaagtcgcgacttgcgagacgatcaacaacaaactcaacaacttgaagcttgctgacgagcgccaagagcggctcgatcatctagcattcaaacatggatccgatactgaggtggaggatatgtacccctgctcgccgacgcagctgggtatcctcatcgcccaagccaagaggccaggtgcttatgtgatgcgcatcctgtttgaactttccatatgcaagggtaacgacaaagtcgatgtcagacgcctccagaaagcttggcaatctgttgtcgagcgtcatgcatcgctgcgcaccgtcttcgtcgaaggcaacacggccgaatccccgtttgagcaaatcgtcattcgacgcgtcaaggcggactttcgcgtcttgcgcatcatgaccgatcatgccagccaggaccgtcttgtatccctgggaaaccacggcggcgccctggccgagttcggtgaactgggtcatcgtcttaccgtcattgccaccccgtctggcaagcactacatccgcctggatatcagccatgctgtcaccgacggtggatcggtcgacatcatgattcgtgatctgaaactcgcgtatggtggaaagttggccaaaggccctggcccgacgtaccgatcgtttatcgacgaaatcctgcaccaaccctcagcccatggtctgcagtactggtcagagtaccttcgcggtgccgagacatgccagttccccactctcctcgacggagacatgtcctcatccccagaaagtgaactaatgtctgcagaagtcaatctttcatctctcacaaaagagattgcagacttctgccgcctccacgaggtgacccttttccacttgttccaggtttgttgggctgttgtcttgcgcgcatatctgaacaaagacgatgtctgtttcggatacacggcctcaggccgcgatgcaccaatcgactccattgagtccatcaccggagctcttgtgagcaccatgctctgccgtgtccaatttgatacgtccaaatcggttacaagcctcatcaaggaggtagcagagcgcaccacagaaagtctcgactttcaacatcattctcttgccgacgtacaaaaggccgctgatcttccaaaagacctcgttttcaatactgtaataggatttcagaccgctctatcgttggactcggatgacacccaagctggccttggggtcaaacagctcagcgtgtattcacctacagagtacgatatagctgtcatcgttgacacggcatccgaagccccttcggtgaatttggactattggacacataggatatcaccagcgcaagccgagatactcgccgatacacttgcccaagtaataacggccgtggtttctcatagcgactacaagattgggcaattggacgtcgtttcaccaaaccaataccagcaaatggttgactggaatatcgattcaactctggaacacggcaacttttctcaaggtacgtcaaccctacatggcatcttccgtgaacgagcgttattatgccctgcggcacccgccgtggtagcccacgacgggcaactgacttatagccaactcgacacactgtcaactcaactttctcatcatttggtatctcgtggtgtggaagtcgagacgctagtgccctactgtttttccaagtcgctatatacggtggtggctattttgggaatactgaaagcagggggtgctgcggttccactagatcccaatcaccccccagatcgcctcgacaccatccttgacgatacgaaggctgccgttattgtcacctccccagaacacagtcatctgttccagcacagacccgccgcaattgtttccgttgatgacgcctttcttcgtctgctcccagaggtctgggaagcccctaacgctgccccgacggccgactcggcagcattggttgtgtacacatcgggtagcactggtgtccccaagggagtcgtgtcgattcatcgagcagtggctacgaattgccaagcttatggcaccgccctccagatgaatgagaagtcccgcgtgcttcagtttgcctctcacactttcgatgctagcatattcgatatgtttgccgcccttcttctcgggggttgtgtgtgcattccgtccgaggaggataggatgaacgatttgcctggatttatcgagcgcacaagctgcaactggattcttgctacgtcaacggtcatgagcatgattacaccgcaacaggcaccaggtctcgaggtccttgtgctgggaggagagccactctcccggacagtgcttgagatgtgggcaccttttgctaccgtattcaacgcgtacggcccggccgagagtgccatcatgtctacgtgctcaccgaagctcactgctgactcccaaccatctctgatcggaaaagctgtgacagggcctgtgtggatcacagacacgtcagatcacgataggctgatgcccatcggatgcattggggagatcagcgtccaaggtccaggagtgtcgcgaggctacctgaacgcgcccgacaaaacctcaaaggtgtttttcgacagtcccgtatggtcgcatccttcgtttgagccgtattctcgattatacagaactggcgatctgggccgcctcaacagcgatggcttcgttgaaattctgggtcgaaaggacaaccaagtcaagttgcggggtcagcggctagagttgggcgaggtggagtaccagatcgcatcgcacgatcaggtcaagcagtgcactgtcgtgaagggcggaaagggtctctgcaaggagcgtcttgtgggtcttatctctctccacggagagccaaagtccatggacactcaaatacgatttgcggaggacaacgacaatgccaaagcccaggtagtctccgaaatccgagaaaagctggctggtgcgctgcccacttacatgatccctacgctttgggctgtcctgcaagagctgccaatcaccacctcgggcaagatcttcaccaagggcctccaacaatgggttgatgagatgagccaggaggccttcgtccgagtcactcgatcatccaaggacggcgagagcgaaaaggatgaagaatcgaggacgaagatgtcggggactgaacaaaagatccagaagatctgggcccgtgttctcaaccttcccatcgaagcgatcacgcggtcaaacacgttcttgtcagtaggtggagattcggtaactgcaatgcgtgttatgtcactctgcagagccgaaagactcctggtcatagttcaggaggtattgcgcgaccagacgctcgctagcttggcaacttgtgctaagactatgggttcgaccaccgcatctgacgacgtcgaagatatatatgacacgccggttggattgagcccaatccagaacctcttcttcgcgacacagggtgtctcgaaccgatggaaccagagcttcatgctcgagttacgaagacctattccactggagagccttcgtggcgcattgaattctctcgtcaagcgacactccatgttgcgtgcccgctttcaaaagacatcgacagggacatggacgcaacagatcccccgaccagatgacggtgtagttcatcagtgcttcaccttcgacactactgatatatcaggagatgtctcaatggtagagcatgcgaacctggtccagtctcgactcaacattgaagatggacgtgttttcacagcaacactgatgagcaagggtgaagagcagtacctttacatggttgcccatcatctggtcattgacctcgtttcctggcggatcatcatccaggacctcgaagatatgctatcgaacaccgcgctcagcaatactctggcgacgcccttccttcagcttactaaagagcgtattgccaagttgggcgacgcaactgcacaccaagatcagagtcgaaatgtcaactctgcggcaaactggagctactgggattttgccgaacagcccaacatcgaagaggatgcccgtaagctgagcttttcgctcgaccaagatgtgtctgggcagcttcttggcgcttgcaacaatgtacttcggacagaacctaccgatctgtttgtcgcggcccttctcaattcgttcgcagaggtcttcacagatcgcaacgtgcctgatgtattcgtagaaggccacggtcgagaccatgaagcgttccaggaaatggatgcgaccggtactgtgggctggttcacagccatttggcctctaaccattccgattacatgggcggggcagcatgtggatctcacgcagacgattcgagccaccaaagacacaagaaggggtcaagagggccctggcaagaccttcctggattctgtcatagccccaggctcatcggatggcgtgccaatggaagtcatctttaactacgaaggacaatatcaacaattggagagttcggatggcctattccggatgctacctctcccagcccctgatatggcttctgatgtgcagcgcactgccttgtttgagatcaccaccgtggttaagggaggccaggtgatgtttgaattcttattcaacgccaagatgggccgtcaagaccagattggcgagtggatcaagaagactgaacttaccttaaccaagcttgccaccgagctgcccaccagatcaaagatgtatagcttgtccgacttccctctcctccaacttgattacgacaatctgcaaacttttacccaggggctagaagttcgacttcaacctttgcgccaacgggatagctcgtttgaaattgaagaggcctacccctgtacaggtatacaggaaggtctactcctatcgcaagggaagcagtcgggaaactatgcagtgcacgagacactggaggttgtttcagcttcaggaacacctattgatgcctcgcgtctaagagacgcttggctccaagtcgtcagtcgacaccctgtgttgcgaaccgtctttgtggaccacccgacggatacggggtatcatgtccaagtggttcttcctcgtgtgatcccggaaatcaacatttcaacgtcccaatcggtcgacgaagctctgttgctgtggaagaacctggggaagaacgattatcccgctgggcaactcatgcaccgctttagcatctgcagcactgctgggggtcgggctctggtcagattcgacttcagccactgtatccttgatgcccagtcttgccaggtgctattcaaggatttgagtcaggcctacgctggaacgctcgacatggacggaagtgtgccacgattcagtcaagttctgcagcatatgcaggggcgtgatcaggcttccgacctgacattttggcaggctgaacttactggcgcggaaccaagcctccttccgaatctttccgactcatctgacgagaccgagcttcatatccagcccctcgaacttggtatatctgccgacgccgtgcacaccttctgcaagcagcacagcgtgagcccgtttgcgatcttgcaggtggcctgggcccgggttctcagcgcctacacggcgatggacaaggtgtcctttgggtacctcagctccggccgcaatgccccaatcaatgggattgaggatatcgccggggttctcgtcaacatgcttgtcacgcatgcgaaggtcgactatagtgtagccgcaattgatgcggcgaaggacatgcatcgcagctacaccgaaagcctggaccaccagtactgctcgctgacagaagtccagcatgctcttgagctcggcggacagccgttgttcaacactgttctcgacttccaacgacctcccgaggaagatgatttggacacagctatctccttacgtcctctggactttcacgatcctacagagttcgctctcacactacactttgagctcaatcataaatctttctctggctacatgtctcactgggcctcgacattaagccaatcatatgcccagagtattggagaggccttggtactggccctacagactatcatggcaaagcccgacatcaaactcggtagcattgacctcatcagcaacaagactctggacaagtacatcgacatcaacactccggcgcccgttgccaagtctgagtgtcttcatgacaccttcagacgccgtgtacaaatggtcccagatgccccagccgtcttctccactgaaatgagtctcacatatgctgagctggatctcctatcaaccaagctggcaaatgagctcttgggcatggacgtgggtcccgagacgaacattctgctgtgctttgagaaatctgcgtggacgattgtgtccatgatggcagtatggaaagctggcggctgtgttgtcgcacttgaccctacacaccctgcttcccgcctaaagggcatagctgaagacgtggctgctcgcataatcctggtctcgccagctactattgacacttgtcgcgaaagcctcgacgcgcagatcagattcatcgttgtcgactcaatgaacttgagcgcaatgattgatgagcggcccaatgtcacgcccgtgaagccggagaatgctgcttacatcaatttcacaagtggcacaaccggcaaaccgaagggtgtggtcattgagcaccaggccattgcaagtaacgtagagcccttgactaaagcggcagacatcaacaacacgacacgagtcctgcagttctcatcgtatgcttgggatgctttctactgcgagaccatcatgccccttctgtctggtggatgtatctgcgttgccaacgaccacgaaaggtcctacgatatcgccgagttcatccgacgcgctaactgcacctgggcgctgtttacaccatcattcgcaaggctcttgacgccttctgacgtgccaaacctccgcaccctactcttgggcggtgaagccatgtcagctgatgatgtgaccacttggtctgagtccgtggtcctcaagaacgcgtatggcccatgcgagtgttctatcgtcgctcttatgcaccccaggattgacgcagcccgcagtgggtcgaacctgggacatcgcttgggccagaccctttgggttgtcgaccctagaaacccacagagacttgcgccagtaggttctccaggtgaactgctcctgggtggtcctcctgtggcgcgagggtatatcaatgacccagcaaagactgccgcggctttcatctcagctcctgcatggttcacatctaacgctgtcctgggcgagcaaggcttcacctctcggttctactgcacgggggatcttgttcgtctcaatgcagatggaaccctcaacttcctcggacggaaggatggagaccaagtgaagctgcgcggccagcgcatggaactcggcgaggttgagcaatgcatcatgacaagtacgtcgacattgaagcaagtagccgccgcggtgatctcccgcaccggccatgccggggacagacagcttgttgccttcgtcaacacgtcggactctaccgacttgaacagcaagccacagacattgcccatgtcggatgagttgctgaagagcctccaggcgctgacgacaaccatggcgaccaaggtaccctctttcatgataccttccatgttcatcccagttaatcgcataccactgagtgcatcgaccaagatcgaccgaaaggctttgaccgagctggcttgccagatccacgaatccagtgtcgctcgttatgcactgactggactttccgaggcttgccccccgacaacgcccatggagataagtcttcacaccctctttgctacagcattgggcgtcgagaccacgtcgctttccatcgccgacaacttcttccaccgtggaggcgactctatcaaggcgatcaagctggtggcggctgcgcgacttgcgaatgtcaatatgacagttgtcgacgtcttcaatcacccaactgttagagagcttgctgctgtggccaacttcatcaactccacaccagacgacccattacgaaatgatagtctcgaagtcttcgtgaaggagatgaagaccgaggtcaccaatgtcgcaattgaagcacacaagttgaaagcgggtcagattcaagatgtgtatccctgcaccgagctgcaggacgcgatgttgtcgataacactgggccagccaggcgcctatgtcctgcaacttgtgacctcaatctccgaagatgcggatcttgaagcctacattgacgcttggaaggccgtgagaaggaataatgacatcttgcggaccagaattgtgagcgcatttgggaagcgctaccaggtggtgtacaatgaccgcatgataatcaagaatgaggagtctcttgacgattatctcgcttctgacaagcagaagcctatggcatatggcgacgcacttgctcgcttcgccctgatctccgaaggagaaaagcattacctagtgtgcaccatgcatcactcaatctacgatggatggtccgtttcactcatcatggatcagctctccgaggcctatcagtggaagccccttcagaagccacagccattctctgaatacgcttggcacgttttcaaccaagatcgcgttgccgcggaacgattctggaccgctgaattggccggcgccacgacatgcgacttccctcgcatgccatcacctacctaccaagccaacgcccgtcagaacgcagcccgaactatggtccttcaacacagccagtcagacatccccctcgcagcaatcatcaaggcagcctgggcgcttctattgggtcaacactccggctcgcgtgatgtcgtctttgcgacaactaacgctggtcgcaacttggatctaccaggcattgctgacatgatcggcccaactattgccacagtacctgtgcgtgttcgctacgattcatcagatagcatcaaagacatggtgaccaaggtgcaccaggggtctctttccatggttccatttgagtatcttgggctgcagcacatccgacatctggacgcctcttgcgaagccgcttgccaattgcgaagcttgctggtcgtccagccaggtgctctcgacgatgctgacattctgcccatccctggtgtcgagaatgtaccagtcgacacaggcaacttctttacgcagcccttggtggtggagtgtaccttgggaaccaacaaagtgaagctctcggctacctacgatgatcatgtcttgagctccgcagagacggatcgcttgcttgctcagcttgagcacttcatccggcaattgagcatgaatatgacaaaaactgtgggcgatatggaccttgtctgccccgaggacaggctcgacctcaactcttggaacgccaatgagccgcccaaggtgaactcgtgcgtctgggatgtgattgcgaagaagacacaagaaacaccaactgccctcgccatctcatcttgggacggtgaaatgacttatgatgaattagacaggcattcgacacgccttgctgctcatctctgtcattcgcttggggtcaaatgtgagacactcgtcccgttggctttcgagaagtcggcatgggctatagtggcgatgctagcggtcgtgaaatccggaggtgcgttcgtatttcttgacgacaaattgccaccagcacgacagcgattcgtggtgacatcaacccagtcccgattcgtcctcagctcgtccaagaatctccccggctggaaccactctcaatggcggagcgtcgaggtcagcgaagcagccataggacagctgcctgaccaagttgcgcccacgcccagctaccagccggacagcatgttatatgccatatatacatcaggtagtactggtaccccaaagggctgtgttgtggagcacgccgcattcctgagcagtgccgctagctacaccaagctcctgaagatgggtccagacacccggtcgcttctgttctcctccttcgcctttgacgtcagcctgatggagtctcttgcggctctcacagtgggtggctgcatctgcatcgccagtgaacaggcatatgaggcaggtgtcgccagcatgattcaggaagccaagcctaactgggcatctctcaccccatcagtggcaagattgatggagcccagagatgtgccttctctcaagacccttgccctcgtaggggagcctttgagccaggaagatgtcaagacatgggctggaagcctccagcttatgaatggttatggcccgacagaatgcgctgttctctgcctcattaatgccaacatgacgacagagaccaaggctacaaataccggctatggcagtggtgggcttacttgggtcgtcgatcccaacgaccctaacattctcatgccaattggctgccccggtgagctcctcatgcaaggtccgatcctagcccgcggctaccttgaccttcctgaggtgacagcatccgcattcattaccggcctcaagtggtcgccccaggatcggttctacaagactggtgacattgtgcgatacagccccgacggatccatccagtgtttcggccgcaaggaccaacagattaaagtccgtggacagagagtcgaactgggtgagattgaggagaacatcgatacactcgactctgtcagccaggcatgtgtggtgttcccaaagtcagggccatacgagaagaagatcgccgctgtcgttgtgctcgaggagttcgcagctcttgactcagctcctctccacctcatttcagccaagcaacaaaaattcgcaggcatcttggtcaaccaaatgcgtgatgccctatcagagaacctgcctagctacatggtcccagaagcctggatcctcgttaccgctctccccatctcaatttcgggtaagagagaccgctcaaccatcggccgatgggtaacggaaatcgacacaaagcaacaagagcagattgacaacctcctcaccccaaccacgattgagtttgccaagacagacgacgagaagaggctacattccattgtgactgaggtgctcaatattgacccttcgcgggcgggtatcaaccagtcattcatgagcttgggtggcgactctatcagcgcgatgcggttaatagccaagtgtaggagccaaggtatcacactcagcctcaagaagttgatgagcagagccagtctttcgcagttggctacttccatgactgcgaatggtagccaaagtgctacagctcaagtcgcctcggacgacaccgggaaaccattcgacctcacaccaattcagcaacactatgtggatcttcacgctcaatgcccgcctctggatacggaaacagagtttaccggccggcgtcgattccaccaaagcttcttcatgaagctcacaaacacagtcagcgcttcgaatgtgactgtcgcggttcgaggacttgttgagcggcacccaatgctgcaagccacattcggacaagattcgaagggtcggtggcaacagactgtaggcagggctcccagtacagttgattgcatctgggtcagcgacgcccaaaccatgcaagcagcgtcagcaacactcgcttccgccaacagcctcgacgtgatcaacggtcccgtattccgtgcagtcttcttttcgaatgatgttgacggtcaaggacaattgcttttcctgacagcgcaccatctggtgattgacttggtgtcatggagaatcgtgctagaggagctagaacagagccttcgtggccagccactacctgagccaactggattgagcttccaatcatggatgaagctccaagccgaccatgcccggctcaacctcagcccaaaatcggccttgccatttgacgtgcatgccagtaacctctcttactggggtatctcgacaccgtctgagaatacttacggcatgaccaagaccgaaagcttcaacttgagcaaggatcttacctctctactgatcggccaatgtcatgaagtacttcgcaccgatgttgtggatgtactcttggcgtccgcgttccgggctttcaccgacatcttcactgagcgagatggtcccacagtgtttgtcgaaggacatggccgggagccttgggatgagtctatcgacattggttcgactgtcgggtggttcactgccctgtgccccgtccacgtacccaagaaacccgaagacacaccccttgagaccctgcgagaggtgaaggatgtgcgccgcagcattccccagaacggatgggcgtacttcgcgtcgaggtacttgaatgctgatggccgagctgccttccaagacgacttgatggcggaaatcatcttcaactacgctggacaatttcaacaactggaggctgacaaccagttgctcgtccctttcgatggcgaactgcctgaagaagcatctgatgttggccctgacacgccgcgatttgctttgatcgaagtgtccgctgtcattcggaatggtcaattgcagatgtcgttcatcttgaacgatcgcatgaagcacgtcgaccgccttggagaatgggtgcgctcggttcagaagaccctcgagactcttatcagcagtctttgcgaccaaccgcagaaatcggtaccgacgctgagcgacttcccactcctacgtaccacccatggcaggcttgatgatcagatatctgaagcatgcgcccggttcggtgcttcggattggactgaaatggaggatatgttcccttgttcaccacttcagaccgcaatgttactgaacaactctatgaatgccgaacaataccgctcatgcgtgatcttcaacataacttcccctacaccaatcaccgccgaggctctgagcgctggttggagcaaggtcgttgcccaccacccttcgctccgaactgtgttcatggaaaccaagacagataatggggcatttgatcaggttgtcttcaagaacctacaacctcaaatcaacgtcctagatatacagggcgatcttgccctcgctactttgaaagcccgcggcccggcggattggtcaaatggcgctcctcatgtcttgaccgtcattccgacatcagctggcgtgctatgcaagcttgagattagtcatgccctgatggatggcgcgtcaatggatctgctatttggcgacctcgtttcggctttggaagacaatctggatgctgccccgagcccttcttaccacggctacattgcgtatctccagaatcgagatgccagtgattccttgacacattggatgagctacctcaaaggcgctaacccttgtctcatctctcctatcaagtctgttacgcagcctgtcgagatcaacgaggtaacttcaatcgaggtaccaatagttgatggcaccgcacaagccctcaacaactttgtcgacgcccatggaattacacaggcgtcggttatccaggctgcctgggcgatggttctgcgtcgctacacccagtcagaagatgtttcgtttggttacgcagttgcaggccgcaatttgccaattgaaggcatcgagaagaccgttggccccttcatcaacgtccttccgtgcagacttgacatgagcgacgacaagctggatatccgtggcttggccaacaaggtccagcaagacttcttcgagagtctccctttccagcatacctcgcttgcggagatgcagcatgagttgcaacttccgacccgtgcgttgttcgacacggtggtgtcaattcagcgttacaatgaggcgtccatgatctcttcaaacggccttcaaatgaatcttctgcacagccgcgatccaacagaatttactctgactctcaacgctgcgtactcgactgacagcttgggctttgttgtgagatactctccgcaaaacggtgttacaagctctagggcggaaagcatcgcgcacacgttccaagctgccctgagtgcgatccttgccaatccagagcagacacttcggaacgttgacctggtcagcaagcatgaccgtgacattattgaccaatggaatgcccatgtacctgaagcagtcgatcaatgcgtgcaagcaagcattaagctgcgtgcagtttcccaacctgcagctgcagcggtatctgcatgggatggagagctctcttaccaagagctggatgatcagacaaaccgtctcgcgggacacctagtacgcaagggtgtcaaagccggcaccattctgccgttctgcttcgagaaatcgatttggaccactgtggcagttctgtctatcttgaagacaggcgcttcgtttctccctttgaacatcaacgaccccgaagagcgtgtcaataccctcctccaggaggctaatgcacaccttgttctcgcgtcgcctagccaatctcggaagtttgaaggagttgtgaatactctggtcatctctgaatcattcattgcggctctcccaccaactagcgataatgagttgcccacaattgccccgagtagcgttgcctatatcctattcacgtcaggttcaactggcaagcctaagggtgttgttattgagcaccggtcgcttgctagtaacatcgcagagcatgcgcctggtctcggactcaattgctccacgcgtgcgctccaattcgcgtcatactctttcgatgcatcgattacagaaactttggcgacgttggtgtcgggaggctgtgtctgtgtcccgtcggaggaggaaaagacggccgacatcgaaggcgcaatgcgacgcatgcgagtcaacttctcgatgctcacgccttcagtggtacaactgttgtcgcctgagaacctcccatctttggaaacactggcgcttgttggagaagctgtaccaaagagcttgatcgagaagtggtccaagaaactcaatcttcttgttggctacgggccgaccgagacggcagtttttgcgtcgattggaagtctacagggccccaacgacgctgggctgattggcaaagctattggcactcaaacgtgggtggtcgaccccgctaaccatgacttactggcgcctatcggttcactgggcgaactcgttctcagtggcccgacgctagccagggggtacctaaacagtcccgacaagacggaacaggccttcaagacaaccccggcctgggctacccctggtataatcaatcgcgtttacaagactggtgatttggtatactatcgcgacgatggcaatctcatgtacgccggccgtattgacggtcaagtcaagatccatggtcaacgtatggaagtcggcgaagtcgagcaccagatccttgagtgccttccagcgcattcggagacagctgttgctgtgctgaaatccgacaaaacctcaattcagcgcttgatggccttcataagcatcccccagatcgaggctgcgcctgcaccctctccgcgactcgccatcttctctgacaaattaagagatgtgcttttgaagttgcagaaggatctgtccgagaaggtcgccacttacatggtcccctctttgtatattcccatgtcatctctgcccaaatcagcagctggcaaggccgaccgtaaagctatgttgctgctcgccgggtcactatcccaggatcagatcgatctgtactccctacgagcctctggtggacaggaagtttcgaccaaggcggagaaatttgtgcagtccgtctgggcatccatgctggggattccggctgataccatatcatcggatgaccatttcttcaaactaggaggcgactccttatctgccatgaaggttgcatctcaaatgcgcagcgagaatgtctttatctccgtcactgagctcttttcgcatccagttttgtcggcgctggccacactcgtggagtcaaagacgggagcggcggaggtcgtatcggtaagcgacgtatcgcccttcgagcttctgtcagacaagcaatgcgtcatcgacaggatcgagaagaccgtgggagtatccatgcatactgtcgaggatgcgtacccagcaacgcctctccaggaagcgatgttcgcaatcacacaaacgagcgattccgcttacgtcaatcgcatggtcttccagattccttcgcacatcgacttgatccgattcaagaaggcctgggaagccgtgtatgctgtcacgccaattctcagaacagcgctcgtcaataccgaggagcatggtacttgccaggttgtatacaaccgtcctgtgccttggtttgaggctgaatgcaacctcgcaagctacttgtacacggattctcaggggcccatctcggaaggcgatgtcctcaccagatacgcaatcatccatgatggtcccgcacattggttcgtctggacagctcaccatgcggcctacgatggctggtcagctggtgcacttttcgatttggtccacgcagcttatgagagtggtgtcgctgttcgaacaccatcttacagcaacttcatccgatacgtctgcaactcgaacgtgaatgatgtcgatgaatactggagcaccacacttgatggttatacccgaacggatttccctccaattccacacccagattaccgagccagcagtgaccaatcagtcgttcactccattcaactacccgccaccagacagaacgccgatatcacccaagcaaccatcgttcgagccgcttgggctttggtggttggcaagcagagtggaactactgatgtcgtcttcggtatgacccagaatggacggaacacacctgtcgcaggcattgaagagatgcaaggaccaaccatcgcaacagtccccgcacgtgttcgaattgacatctcaaagagcatatcaacatacctgcaagagatgcagcagcaagcagccggctcgatcccttatgagcaagccggactccagaacatccgcactttgaaccaaggagcagaagaggcctgcgacttcaacaacctgctcgtcattcaacccaagggcgaagcgggcggagaagtctggctagagcctgttactggagaactgaacaaattcgacaatttcccactgactattgagtgtgcgattggaaaggagggacatatggagctcttggcccacctcgactcccatctgctctccaaactccaggttgagcgcatcctcttccaatttagccatgtgatccgtcagctttctgaggccaacactgagactctactctctgatgttgagttcttcactcctgaggatcgtgctaccatcttcggtgaatggaactccaacatcccaggcactgttgaagcatctctacaagacagattcgaagagaatgcccgcgaatatccacagaagcaagcaatttactcttgggatgcgttaatgacatattcggagctcgatcagatgtcaacacatcttgcacacaagcttatcgatgcgggtgtgaaacctggggagttcgttccactctgtttcgagaagtcagcgtgggccgttgtttctatgctggcggcgatgaaggctggagcagcctttgtgtcaattgatcccgagcatccagagaatcgtcgtcaggatataatcgaccagaccaatgcccaagtcatgctagcttccgggacagccccacctctatcggtcgaccagaccttggtcgtctcggccgatacactatccggctatcgacaaactaaggactcatctacattgccccccaaagaccctagcaggcttgcctaccttatctttaccagcggtagcactggccgcagcaagggggttatgatgtcacaccgagctgtctgctctagtgctcagatgttagacttggaaggtcttggtttcgcccgggactccagagtgtaccagttcccctcttacgtcttcgacgcatctatcttcaacatcttcgggactctgtctgcaggagccacgctggtggtaccagctgacgaagacaggcttggtgccaacctagccaagtcaatgcgtgacgcggaagtcaccaaagccatcataaccccgaccgtcagtcggttactccaaccagaggatgtaccaactttgcgcaccctcaaactgggaggcgatgctgtataccaagaggatgtcgacaagtgggcttcgaaagtacacatgctgcagggttacggcccagcagaagccactgtctttgtcacctggacagagataactccccaaagcgtacctaacaatgtcggacctgccagcagatccaggctttgggtggtggaccccgacaaccacgacaagctgacacccataggcatggttggcgaaatgctcattgagggccccaccctcgccaacgggtacctgaatgacgaagagaaaaccgctgcggcattcattacaagcccggcatggtgtcataatgagtcaagacgattttacaagacgggagacctagtccgctatgccgataatggttcgttcttgttcatcggtcgcaaagatgcccaagtcaaaatcaatggccagagagtcgagctcggtgagatcgagaaccatctcttcaacatgttgccatcatcccaattcgtcgctctcgttccaaaagccggtccctgcaagaacgttctcactgtactagtggccgcgacaaccgctgactcagtcaagagacttgagtttatcgaagatgcctccacgaaggagtctttgagcaaggctgtctccgaactgcgtgaagccatgccttcccgtgtccccaagtacatggtaccacgtacatggttcgcggtcagcaggctacctcttacttcgtcgggcaaagtcgaccgcaagaatatccagcgctggctggaagagcttgaccccgacgtcgcacggatggaaacactttctcttaagcacgatgccgccgctacaatacccacgaccaagacggagagtgttgtacagcaggctattggccgagtcctcaatctcgcacctgagaacatcccgctcgacaagaccttcatggccttgggaggtgactcgatcactggaatgcaactggtatcccgacttcaatcgtcaagcatcgtgctcagcgtgcgacagctgctccaaagccataccatccgacaccttgcgtcgcttgcacaacccgaaggcgaacgggtggagctagcggatgaagtggttggcgtgcctttcgatttatcgccaattcagaccatgctgattgacaccatcttgcccgatgcaatggcacctcggcgtttcaaccaaagcttccttgtccgcttgagtcacgaggtggatacagggaaattgcgagatgccttcacttaccttgtatctcgccattccatgttgaggtcacgttttgagaagacctcagatggccgctggagccagagaataatgccagagtcagacagcgcataccagctctcaatccatggaacgtctcatgatctgggcaagatcatggcggatagccagagcggcttagatgtcttccaaggaccagtttttgcaggtgatgtgtgcgccgacaatgaaggcaagcaagtcttgttcatggtcgcacatcatcttgccgttgatttggtatcatggcgtatcatccttaccgacctcgaggagtacctgacaacagacaacttcaaggagaaacacaagccgttgtcgttccagacatggctatcactgcaacaaaagaaggcggaggatattcgaccggacactgctcttccctttaccccaccacaagcgcctatcaactactggggggcactcactgaatccaacacctttagcgccgtggaggaacagtcgttcacactcggtgcggatctaacatcgcacctactcaaggactgcagccaagtgctaggaactgacacggttgatatcctcatcggcgccttgacccatgcattctcgaaaacgttccgggatcgcgacctgccgactgtcttcaccgaataccacggtcgcgaatcttgggatgattcaattgatatctcatctaccgtgggttggttcaccaccctggcgcctgtgcatgtcactagccatggcgacgatggtatcaaacctttcgtccggaaagtgaaggattgtcggcgaaagaccccaatgaacgggtggtcttactttgcttcgcgcttcttgagccccgaaggcagagagcgattcgcccagagcggacccatggaaattgtgctgaacttcgtcggacagtaccagcaactcgaacgagccgaaggactcttcgagatggtccctcgcgatgactttggctcggacaccgtcgacgtcagcccagatctggaacgctcctcaatcttcgagatttcgtctggcgtgatgaatggccatctgaatttctcaatgatgtggccacgaagtcttcgcaaccaaagccagattcgacaatggctgaccaataccgaacaatctctgatcgaagctatcaaagaccttcaaggtctcactagagagttgactgtcagcgacttccctctgctctctatggcagatgacgagttcgttagctttgtggaggactctttgccaaagcaccacatcgctgttggggacgttgaagatatttaccctgcgacaccactgcaagaagccttaatgatgtcacgaagtatcgactccgaattatacaccgtacacgccatctaccagctcagctgcaaggatggcgagcctgttgatacggagagactcaagaccgcctggaactccgtggtcagccgtcatcagagtctaagaactgtcttcgttggcgttgtgtccgaccacagtgcactcaatcaagtcgtactgaaggcgatgcctaatacgacagtggaattgacttgcgatactgacgaccaggccctggaaatgctcgaaaagccaaacacactgagcccaagtctagcctgggaggtagaaaccccagcgcagctgatcatctgccatgctatagcatccggcacaacttactgcaagatcgccatcaaccatgcggtcgccgacggtggctccaatggtatcctcatccgggacatcggcctggcctacgaaggcctcctctctgcagagcgtggtccgctatacagtcaatatgtctcttacctccagagccgcgacagacaagcaagcctcgactactggggcgaatatctgcacggtcttgaaccgtgcatggtcaatggtggcacacgctctgggaagaccaaggaactccgtgatcttgaagtcgagatcccacatcccgacaaattgagagctttctctgctgcgaggtctgtcacactcgccaacgtcttccaagcagcatgggccttagtactgcgcaacttcgccggcacagaccaagtctgctatggttacctcaacgccggtcgcgattcacccgtcgacgggattcgcgatgcggtcggactttttgtgaatatgctcgtgagccgtgtcgatttcaccgagaaccttacagcaggagagctagttaagcaggttcacaatgactacatcgacgccctcgatcaccagaactgttcactggccgacatgcaacggatcgccggccatccgctattcaacagcatcatgtcgtaccaggtcgcttcatcttcgtccgaggagacctctagcgatctcaactttgcccaagtcagcgctcatgaccccactgagtaccctcttagtatcagtgtcgcggtttccgacaagtcgatcttcgtcacccttagctactacacagatctggtttcagattttcaggcgcagagtgtggccaccagcttcgagaaggcgctcggcgggttcactgaagcaacactcgaccttcccgcgcgcaacattccaattctcggcgataaccacatctccaagatccaagagtggaacgctgaggggcccaaagcggttgaaggatgcattcaccagatattcgaggaccaagtcaaggcgcaccctgacgccatggcggtgtgttcaaccgagatcagctggacctacagccagttggatgtgctttccactcgcctggcacatcgcctcgccatcctgggtattggacccggcaccatagtcccgtactgcttcgctgcctccccatggacggtggtgatcatgatggctattctcaaatcgggaggtgcctgcgtggctctggatcccaaacatcccatcgatcgtctgcacggcatcatcgaagacgttgacgccgatattgtcattacttcgtctcgccacgcttccaagttcttaccggtggcggcggtattggcgctcggtccggaagaaattactcgcattggcaaaaccgcacccagacggcttggcacgttgccaatcgctcaaccaagtgacgtcgcagttgttcagttcactagtggcagtaccggcaaacccaagggaatcttgctcgaacaccagtcgttatgcaccagtagtgcctactatggtgctgcgatgggctatggcccaggctcgcgcgtcatccaattctcttcctacacctttgatgtcagcatcagcgatatcttcttctctttgatgagaggcggtgctgtttgcgtgcccactgaggatgagaagctcaacgacctggcggactttatcaacaggctcgaggtcaacaccgccgacttgactccatcggtcctcgaagcaacactatctcccgaggcagtcccgaccttgaagacgatatgtctcggaggtgaagcagtaaagcaagacaatctcaatgtatgggctgatcatgtggccctacacaattactacggtccctctgagtgctcagtcgcctgtgttggacgatccaatctctcttccaccgatctggctgccaacatcggagtcggatgtggtgcgctgacatgggtcgttgagagttccaacgcccacaagctcgtaccgcttggaactgtcggcgaacttgtgctcgaaggaccgctcctcgcacaaggttacttgaaactgcccgagaagactaccgaagtgttcttggagaatgttcaatggccaggcttcaaggaacccaaacgcctataccgaacgggcgatctcgtgaggtacaatccagacggaagcctcgactaccttggaaggaaggatacgcaagtcaagattagaggtcaacgtgttgagactggcgagatcgaacaccacgccgccctctgctgtcctccttcaatctctcaactcgcagtggaggcagtaacattgccagggagaagtggtttggcacttgccatgttcgtggcagtcggcgaccagccagatgcctgcatggaagttggcttccctcccgagctacactctgctctccttggcatgaaaaacagtttggcagcacatgtcccatcgtacatgcttccttcaatcttcgttccccttcgcgagattccccggagtctcgctgggaaacgcgaccgtagaactcttcaagagttcgtatcagcactcccggctgacatgattcgaacattctctctcgccgacgaggtcaccaccaagagagagccatcgactgcaattgagcgcagtctacgcggggtctggtccaaggtcctccgcgtgaaagagacggatattggtattgatgacagctttttcaaattgggcggtgactccatctccgccatgcgacttgctaccgctgcgaccaagagcggcctgcaagtttccgtgctggacatcctaacacacaagacaatttccaagattgccgcgctcgccgagaccaaggtcgatgaagcatacgccacccagagcgtcaccagctccgagcgcagtggtgaaaccttgggcaagtcgtctttcttggatgagatgcccgctcccttcgatcccaatgaggtcgaggtcatactgcccacaactagcgcccagaaagaggtgcttcaagcatgcaaggacagcccgaactctgactactaccacatttcccaaatcattgaggttcttccacaagaagtgggaggacgcaaggtagatgcggagctgctacacggagcctggcaacaggtcgtcgaccgccacccttcccttcgcaccgttctcatcaacaatgtaggacaagttatcctcaaacaacacatggccaaggtctcgcacattgaggtggaagaatcagacgacctgcaaaattacccaggccccgtaccactgtgggctgagttcagtccaatgcaccatgtcaccatcgcccagacaccctcgggtagaattctgtgtcgcattaacatacaccatgccatcactgatggcttttctctgatgaccatgttcgaggaattggccagcgcatactctggtacactcgactttggacccggaccgtcgctccaagagtacatctcccaggtcgagtccttggatcgcaaggcctcagcatcttactggaagaccgagatggacgcaatccaaccccggtccttcccacttaccaaatcaatcatcgcgactaacgacgccgcgcgcgctgatctacaagagctgcactgccagatcaatggcgaaaagctgctcgacttctgcctcgataacgagacgaccataccctcactagtgtacgcggcctggggtctcgtcctccaagccatgtgcacagatgatgtcaacggcggacaggacatcctgttcgcctatctctcgagcgcccgtgctatcatcccctcagaggccctgggattcctcgtcaacatcgtgccccaacgcgtccgcgccgcagacctagatgcggcatcactgccgcgcttcgtgcgcaacattcacgcgtccgtgctcgagtcgctacctcactcgcacattgcgccctctgagctcgggcttaaaatcaataccttggtaaacatgcgcaagttcgatgacgcggacatcctcgctgcattccatggcgatgaggctatggagcaactgagcagcgacggtcttgaacctgctgtcaagaaccaacaaccacaacagaaaccacatgaaatcacttttgatgcattcccttcgcccgaccccatggcatacgatgtcgtggtcgcggtctcggaactgggcggcgggacactggatgtcacgctctcgttctggaactcggtcgtggacgaaggcgacgcgcagcgcgctctagattgcttgaagagcgtgctgggcatgatgggacgggagggggagactatgacttgtgctgaagtcgtgggccagttggctgtctgatatgagacaacgaaaaaaaaaaaaaaaaaaaaaaaaaaacatgcacgatgacatttcctttgtagaatagggggaggtgttcaggatactgattgggattggttcttctttggtcgttacgtctcctacgttggtaaaggaagaaaggagttctggttcgtgggttatcttggttctacatctagttaatctctttcatctttcctttgaatattcatacactttaacaaccttc

SEQ ID NO: 4 - protein, 8493 aa, unidentified
MGVSSDTTSHLLEVKTPAMDAGASPKTRRRRALSGAEAQQEFGASAAANLLQQIQAAWSKALGLPTELIGTTQSFASLGGDSVAAMKVVSILSLAGIRVLVKDVMRGKSIQGLASAAKQVAAPATTAASQPPPQSQPEPHSLKEVATCETINNKLNNLKLADERQERLDHLAFKHGSDTEVEDMYPCSPTQLGILIAQAKRPGAYVMRILFELSICKGNDKVDVRRLQKAWQSVVERHASLRTVFVEGNTAESPFEQIVIRRVKADFRVLRIMTDHASQDRLVSLGNHGGALAEFGELGHRLTVIATPSGKHYIRLDISHAVTDGGSVDIMIRDLKLAYGGKLAKGPGPTYRSFIDEILHQPSAHGLQYWSEYLRGAETCQFPTLLDGDMSSSPESELMSAEVNLSSLTKEIADFCRLHEVTLFHLFQVCWAVVLRAYLNKDDVCFGYTASGRDAPIDSIESITGALVSTMLCRVQFDTSKSVTSLIKEVAERTTESLDFQHHSLADVQKAADLPKDLVFNTVIGFQTALSLDSDDTQAGLGVKQLSVYSPTEYDIAVIVDTASEAPSVNLDYWTHRISPAQAEILADTLAQVITAVVSHSDYKIGQLDVVSPNQYQQMVDWNIDSTLEHGNFSQGTSTLHGIFRERALLCPAAPAVVAHDGQLTYSQLDTLSTQLSHHLVSRGVEVETLVPYCFSKSLYTVVAILGILKAGGAAVPLDPNHPPDRLDTILDDTKAAVIVTSPEHSHLFQHRPAAIVSVDDAFLRLLPEVWEAPNAAPTADSAALVVYTSGSTGVPKGVVSIHRAVATNCQAYGTALQMNEKSRVLQFASHTFDASIFDMFAALLLGGCVCIPSEEDRMNDLPGFIERTSCNWILATSTVMSMITPQQAPGLEVLVLGGEPLSRTVLEMWAPFATVFNAYGPAESAIMSTCSPKLTADSQPSLIGKAVTGPVWITDTSDHDRLMPIGCIGEISVQGPGVSRGYLNAPDKTSKVFFDSPVWSHPSFEPYSRLYRTGDLGRLNSDGFVEILGRKDNQVKLRGQRLELGEVEYQIASHDQVKQCTVVKGGKGLCKERLVGLISLHGEPKSMDTQIRFAEDNDNAKAQVVSEIREKLAGALPTYMIPTLWAVLQELPITTSGKIFTKGLQQWVDEMSQEAFVRVTRSSKDGESEKDEESRTKMSGTEQKIQKIWARVLNLPIEAITRSNTFLSVGGDSVTAMRVMSLCRAERLLVIVQEVLRDQTLASLATCAKTMGSTTASDDVEDIYDTPVGLSPIQNLFFATQGVSNRWNQSFMLELRRPIPLESLRGALNSLVKRHSMLRARFQKTSTGTWTQQIPRPDDGVVHQCFTFDTTDISGDVSMVEHANLVQSRLNIEDGRVFTATLMSKGEEQYLYMVAHHLVIDLVSWRIIIQDLEDMLSNTALSNTLATPFLQLTKERIAKLGDATAHQDQSRNVNSAANWSYWDFAEQPNIEEDARKLSFSLDQDVSGQLLGACNNVLRTEPTDLFVAALLNSFAEVFTDRNVPDVFVEGHGRDHEAFQEMDATGTVGWFTAIWPLTIPITWAGQHVDLTQTIRATKDTRRGQEGPGKTFLDSVIAPGSSDGVPMEVIFNYEGQYQQLESSDGLFRMLPLPAPDMASDVQRTALFEITTVVKGGQVMFEFLFNAKMGRQDQIGEWIKKTELTLTKLATELPTRSKMYSLSDFPLLQLDYDNLQTFTQGLEVRLQPLRQRDSSFEIEEAYPCTGIQEGLLLSQGKQSGNYAVHETLEVVSASGTPIDASRLRDAWLQVVSRHPVLRTVFVDHPTDTGYHVQVVLPRVIPEINISTSQSVDEALLLWKNLGKNDYPAGQLMHRFSICSTAGGRALVRFDFSHCILDAQSCQVLFKDLSQAYAGTLDMDGSVPRFSQVLQHMQGRDQASDLTFWQAELTGAEPSLLPNLSDSSDETELHIQPLELGISADAVHTFCKQHSVSPFAILQVAWARVLSAYTAMDKVSFGYLSSGRNAPINGIEDIAGVLVNMLVTHAKVDYSVAAIDAAKDMHRSYTESLDHQYCSLTEVQHALELGGQPLFNTVLDFQRPPEEDDLDTAISLRPLDFHDPTEFALTLHFELNHKSFSGYMSHWASTLSQSYAQSIGEALVLALQTIMAKPDIKLGSIDLISNKTLDKYIDINTPAPVAKSECLHDTFRRRVQMVPDAPAVFSTEMSLTYAELDLLSTKLANELLGMDVGPETNILLCFEKSAWTIVSMMAVWKAGGCVVALDPTHPASRLKGIAEDVAARIILVSPATIDTCRESLDAQIRFIVVDSMNLSAMIDERPNVTPVKPENAAYINFTSGTTGKPKGVVIEHQAIASNVEPLTKAADINNTTRVLQFSSYAWDAFYCETIMPLLSGGCICVANDHERSYDIAEFIRRANCTWALFTPSFARLLTPSDVPNLRTLLLGGEAMSADDVTTWSESVVLKNAYGPCECSIVALMHPRIDAARSGSNLGHRLGQTLWVVDPRNPQRLAPVGSPGELLLGGPPVARGYINDPAKTAAAFISAPAWFTSNAVLGEQGFTSRFYCTGDLVRLNADGTLNFLGRKDGDQVKLRGQRMELGEVEQCIMTSTSTLKQVAAAVISRTGHAGDRQLVAFVNTSDSTDLNSKPQTLPMSDELLKSLQALTTTMATKVPSFMIPSMFIPVNRIPLSASTKIDRKALTELACQIHESSVARYALTGLSEACPPTTPMEISLHTLFATALGVETTSLSIADNFFHRGGDSIKAIKLVAAARLANVNMTVVDVFNHPTVRELAAVANFINSTPDDPLRNDSLEVFVKEMKTEVTNVAIEAHKLKAGQIQDVYPCTELQDAMLSITLGQPGAYVLQLVTSISEDADLEAYIDAWKAVRRNNDILRTRIVSAFGKRYQVVYNDRMIIKNEESLDDYLASDKQKPMAYGDALARFALISEGEKHYLVCTMHHSIYDGWSVSLIMDQLSEAYQWKPLQKPQPFSEYAWHVFNQDRVAAERFWTAELAGATTCDFPRMPSPTYQANARQNAARTMVLQHSQSDIPLAAIIKAAWALLLGQHSGSRDVVFATTNAGRNLDLPGIADMIGPTIATVPVRVRYDSSDSIKDMVTKVHQGSLSMVPFEYLGLQHIRHLDASCEAACQLRSLLVVQPGALDDADILPIPGVENVPVDTGNFFTQPLVVECTLGTNKVKLSATYDDHVLSSAETDRLLAQLEHFIRQLSMNMTKTVGDMDLVCPEDRLDLNSWNANEPPKVNSCVWDVIAKKTQETPTALAISSWDGEMTYDELDRHSTRLAAHLCHSLGVKCETLVPLAFEKSAWAIVAMLAVVKSGGAFVFLDDKLPPARQRFVVTSTQSRFVLSSSKNLPGWNHSQWRSVEVSEAAIGQLPDQVAPTPSYQPDSMLYAIYTSGSTGTPKGCVVEHAAFLSSAASYTKLLKMGPDTRSLLFSSFAFDVSLMESLAALTVGGCICIASEQAYEAGVASMIQEAKPNWASLTPSVARLMEPRDVPSLKTLALVGEPLSQEDVKTWAGSLQLMNGYGPTECAVLCLINANMTTETKATNTGYGSGGLTWVVDPNDPNILMPIGCPGELLMQGPILARGYLDLPEVTASAFITGLKWSPQDRFYKTGDIVRYSPDGSIQCFGRKDQQIKVRGQRVELGEIEENIDTLDSVSQACVVFPKSGPYEKKIAAVVVLEEFAALDSAPLHLISAKQQKFAGILVNQMRDALSENLPSYMVPEAWILVTALPISISGKRDRSTIGRWVTEIDTKQQEQIDNLLTPTTIEFAKTDDEKRLHSIVTEVLNIDPSRAGINQSFMSLGGDSISAMRLIAKCRSQGITLSLKKLMSRASLSQLATSMTANGSQSATAQVASDDTGKPFDLTPIQQHYVDLHAQCPPLDTETEFTGRRRFHQSFFMKLTNTVSASNVTVAVRGLVERHPMLQATFGQDSKGRWQQTVGRAPSTVDCIWVSDAQTMQAASATLASANSLDVINGPVFRAVFFSNDVDGQGQLLFLTAHHLVIDLVSWRIVLEELEQSLRGQPLPEPTGLSFQSWMKLQADHARLNLSPKSALPFDVHASNLSYWGISTPSENTYGMTKTESFNLSKDLTSLLIGQCHEVLRTDVVDVLLASAFRAFTDIFTERDGPTVFVEGHGREPWDESIDIGSTVGWFTALCPVHVPKKPEDTPLETLREVKDVRRSIPQNGWAYFASRYLNADGRAAFQDDLMAEIIFNYAGQFQQLEADNQLLVPFDGELPEEASDVGPDTPRFALIEVSAVIRNGQLQMSFILNDRMKHVDRLGEWVRSVQKTLETLISSLCDQPQKSVPTLSDFPLLRTTHGRLDDQISEACARFGASDWTEMEDMFPCSPLQTAMLLNNSMNAEQYRSCVIFNITSPTPITAEALSAGWSKVVAHHPSLRTVFMETKTDNGAFDQVVFKNLQPQINVLDIQGDLALATLKARGPADWSNGAPHVLTVIPTSAGVLCKLEISHALMDGASMDLLFGDLVSALEDNLDAAPSPSYHGYIAYLQNRDASDSLTHWMSYLKGANPCLISPIKSVTQPVEINEVTSIEVPIVDGTAQALNNFVDAHGITQASVIQAAWAMVLRRYTQSEDVSFGYAVAGRNLPIEGIEKTVGPFINVLPCRLDMSDDKLDIRGLANKVQQDFFESLPFQHTSLAEMQHELQLPTRALFDTVVSIQRYNEASMISSNGLQMNLLHSRDPTEFTLTLNAAYSTDSLGFVVRYSPQNGVTSSRAESIAHTFQAALSAILANPEQTLRNVDLVSKHDRDIIDQWNAHVPEAVDQCVQASIKLRAVSQPAAAAVSAWDGELSYQELDDQTNRLAGHLVRKGVKAGTILPFCFEKSIWTTVAVLSILKTGASFLPLNINDPEERVNTLLQEANAHLVLASPSQSRKFEGVVNTLVISESFIAALPPTSDNELPTIAPSSVAYILFTSGSTGKPKGVVIEHRSLASNIAEHAPGLGLNCSTRALQFASYSFDASITETLATLVSGGCVCVPSEEEKTADIEGAMRRMRVNFSMLTPSVVQLLSPENLPSLETLALVGEAVPKSLIEKWSKKLNLLVGYGPTETAVFASIGSLQGPNDAGLIGKAIGTQTWVVDPANHDLLAPIGSLGELVLSGPTLARGYLNSPDKTEQAFKTTPAWATPGIINRVYKTGDLVYYRDDGNLMYAGRIDGQVKIHGQRMEVGEVEHQILECLPAHSETAVAVLKSDKTSIQRLMAFISIPQIEAAPAPSPRLAIFSDKLRDVLLKLQKDLSEKVATYMVPSLYIPMSSLPKSAAGKADRKAMLLLAGSLSQDQIDLYSLRASGGQEVSTKAEKFVQSVWASMLGIPADTISSDDHFFKLGGDSLSAMKVASQMRSENVFISVTELFSHPVLSALATLVESKTGAAEVVSVSDVSPFELLSDKQCVIDRIEKTVGVSMHTVEDAYPATPLQEAMFAITQTSDSAYVNRMVFQIPSHIDLIRFKKAWEAVYAVTPILRTALVNTEEHGTCQVVYNRPVPWFEAECNLASYLYTDSQGPISEGDVLTRYAIIHDGPAHWFVWTAHHAAYDGWSAGALFDLVHAAYESGVAVRTPSYSNFIRYVCNSNVNDVDEYWSTTLDGYTRTDFPPIPHPDYRASSDQSVVHSIQLPATRQNADITQATIVRAAWALVVGKQSGTTDVVFGMTQNGRNTPVAGIEEMQGPTIATVPARVRIDISKSISTYLQEMQQQAAGSIPYEQAGLQNIRTLNQGAEEACDFNNLLVIQPKGEAGGEVWLEPVTGELNKFDNFPLTIECAIGKEGHMELLAHLDSHLLSKLQVERILFQFSHVIRQLSEANTETLLSDVEFFTPEDRATIFGEWNSNIPGTVEASLQDRFEENAREYPQKQAIYSWDALMTYSELDQMSTHLAHKLIDAGVKPGEFVPLCFEKSAWAVVSMLAAMKAGAAFVSIDPEHPENRRQDIIDQTNAQVMLASGTAPPLSVDQTLVVSADTLSGYRQTKDSSTLPPKDPSRLAYLIFTSGSTGRSKGVMMSHRAVCSSAQMLDLEGLGFARDSRVYQFPSYVFDASIFNIFGTLSAGATLVVPADEDRLGANLAKSMRDAEVTKAIITPTVSRLLQPEDVPTLRTLKLGGDAVYQEDVDKWASKVHMLQGYGPAEATVFVTWTEITPQSVPNNVGPASRSRLWVVDPDNHDKLTPIGMVGEMLIEGPTLANGYLNDEEKTAAAFITSPAWCHNESRRFYKTGDLVRYADNGSFLFIGRKDAQVKINGQRVELGEIENHLFNMLPSSQFVALVPKAGPCKNVLTVLVAATTADSVKRLEFIEDASTKESLSKAVSELREAMPSRVPKYMVPRTWFAVSRLPLTSSGKVDRKNIQRWLEELDPDVARMETLSLKHDAAATIPTTKTESVVQQAIGRVLNLAPENIPLDKTFMALGGDSITGMQLVSRLQSSSIVLSVRQLLQSHTIRHLASLAQPEGERVELADEVVGVPFDLSPIQTMLIDTILPDAMAPRRFNQSFLVRLSHEVDTGKLRDAFTYLVSRHSMLRSRFEKTSDGRWSQRIMPESDSAYQLSIHGTSHDLGKIMADSQSGLDVFQGPVFAGDVCADNEGKQVLFMVAHHLAVDLVSWRIILTDLEEYLTTDNFKEKHKPLSFQTWLSLQQKKAEDIRPDTALPFTPPQAPINYWGALTESNTFSAVEEQSFTLGADLTSHLLKDCSQVLGTDTVDILIGALTHAFSKTFRDRDLPTVFTEYHGRESWDDSIDISSTVGWFTTLAPVHVTSHGDDGIKPFVRKVKDCRRKTPMNGWSYFASRFLSPEGRERFAQSGPMEIVLNFVGQYQQLERAEGLFEMVPRDDFGSDTVDVSPDLERSSIFEISSGVMNGHLNFSMMWPRSLRNQSQIRQWLTNTEQSLIEAIKDLQGLTRELTVSDFPLLSMADDEFVSFVEDSLPKHHIAVGDVEDIYPATPLQEALMMSRSIDSELYTVHAIYQLSCKDGEPVDTERLKTAWNSVVSRHQSLRTVFVGVVSDHSALNQVVLKAMPNTTVELTCDTDDQALEMLEKPNTLSPSLAWEVETPAQLIICHAIASGTTYCKIAINHAVADGGSNGILIRDIGLAYEGLLSAERGPLYSQYVSYLQSRDRQASLDYWGEYLHGLEPCMVNGGTRSGKTKELRDLEVEIPHPDKLRAFSAARSVTLANVFQAAWALVLRNFAGTDQVCYGYLNAGRDSPVDGIRDAVGLFVNMLVSRVDFTENLTAGELVKQVHNDYIDALDHQNCSLADMQRIAGHPLFNSIMSYQVASSSSEETSSDLNFAQVSAHDPTEYPLSISVAVSDKSIFVTLSYYTDLVSDFQAQSVATSFEKALGGFTEATLDLPARNIPILGDNHISKIQEWNAEGPKAVEGCIHQIFEDQVKAHPDAMAVCSTEISWTYSQLDVLSTRLAHRLAILGIGPGTIVPYCFAASPWTVVIMMAILKSGGACVALDPKHPIDRLHGIIEDVDADIVITSSRHASKFLPVAAVLALGPEEITRIGKTAPRRLGTLPIAQPSDVAVVQFTSGSTGKPKGILLEHQSLCTSSAYYGAAMGYGPGSRVIQFSSYTFDVSISDIFFSLMRGGAVCVPTEDEKLNDLADFINRLEVNTADLTPSVLEATLSPEAVPTLKTICLGGEAVKQDNLNVWADHVALHNYYGPSECSVACVGRSNLSSTDLAANIGVGCGALTWVVESSNAHKLVPLGTVGELVLEGPLLAQGYLKLPEKTTEVFLENVQWPGFKEPKRLYRTGDLVRYNPDGSLDYLGRKDTQVKIRGQRVETGEIEHHAALCCPPSISQLAVEAVTLPGRSGLALAMFVAVGDQPDACMEVGFPPELHSALLGMKNSLAAHVPSYMLPSIFVPLREIPRSLAGKRDRRTLQEFVSALPADMIRTFSLADEVTTKREPSTAIERSLRGVWSKVLRVKETDIGIDDSFFKLGGDSISAMRLATAATKSGLQVSVLDILTHKTISKIAALAETKVDEAYATQSVTSSERSGETLGKSSFLDEMPAPFDPNEVEVILPTTSAQKEVLQACKDSPNSDYYHISQIIEVLPQEVGGRKVDAELLHGAWQQVVDRHPSLRTVLINNVGQVILKQHMAKVSHIEVEESDDLQNYPGPVPLWAEFSPMHHVTIAQTPSGRILCRINIHHAITDGFSLMTMFEELASAYSGTLDFGPGPSLQEYISQVESLDRKASASYWKTEMDAIQPRSFPLTKSIIATNDAARADLQELHCQINGEKLLDFCLDNETTIPSLVYAAWGLVLQAMCTDDVNGGQDILFAYLSSARAIIPSEALGFLVNIVPQRVRAADLDAASLPRFVRNIHASVLESLPHSHIAPSELGLKINTLVNMRKFDDADILAAFHGDEAMEQLSSDGLEPAVKNQQPQQKPHEITFDAFPSPDPMAYDVVVAVSELGGGTLDVTLSFWNSVVDEGDAQRALDCLKSVLGMMGREGETMTCAEVVGQLAV

SEQ ID NO: 5 - DNA, 366 bp, synthetic construct
  source 1..366: mol_type = other DNA
gaagttcagctgctggaaagcggcggcggcctggttcagccgggcgggtccctgcgcctgagctgcgcggctagcggctttcgggattccgatgaagatatgggctgggtgcgccaggcacctggtaaaggcctggaatgggtgagctccatttactcttacaacgggtctacctattatgcagatagcgtgaaaggccgctttaccatctcgagagataattcgaaaaacaccctgtatctgcaaatgaacagcctgcgtgcggaagatactgcagtttattattgcgcgagcgaccgtggtggttacaactctttcgactactggggccagggtaccctggtgactgtgagcagcgcggccgca

SEQ ID NO: 6 - protein, 122 aa, synthetic construct
EVQLLESGGGLVQPGGSLRLSCAASGFRDSDEDMGWVRQAPGKGLEWVSSIYSYNGSTYYADSVKGRFTISRDNSKNTLYLQMNSLRAEDTAVYYCASDRGGYNSFDYWGQGTLVTVSSAAA

SEQ ID NO: 7 - protein, 10 aa, synthetic construct
GFRDSDEDMG

SEQ ID NO: 8 - protein, 16 aa, synthetic construct
IYSYNGSTYYADSVKG

SEQ ID NO: 9 - protein, 10 aa, synthetic construct
DRGGYNSFDY

SEQ ID NO: 10 - DNA, 366 bp, synthetic construct
  source 1..366: mol_type = other DNA
gaagttcagctgctggaaagcggcggcggcctggttcagccgggcgggtccctgcgcctgagctgcgcggctagcggctttcgggattccgatgaagatatgggctgggtgcgccaggcacctggtaaaggcctggaatgggtgagctccattgaaactccggacgggtctacctattatgcagatagcgtgaaaggccgctttaccatctcgagagataattcgaaaaacaccctgtatctgcaaatgaacagcctgcgtgcggaagatactgcagtttattattgcgcgagccagccggtttactcttaccatttcgactactggggccagggtaccctggtgactgtgagcagcgcggccgca

SEQ ID NO: 11 - protein, 122 aa, synthetic construct
EVQLLESGGGLVQPGGSLRLSCAASGFRDSDEDMGWVRQAPGKGLEWVSSIETPDGSTYYADSVKGRFTISRDNSKNTLYLQMNSLRAEDTAVYYCASQPVYSYHFDYWGQGTLVTVSSAAA

SEQ ID NO: 12 - protein, 10 aa, synthetic construct
GFRDSDEDMG

SEQ ID NO: 13 - protein, 16 aa, synthetic construct
IETPDGSTYYADSVKG

SEQ ID NO: 14 - protein, 10 aa, synthetic construct
QPVYSYHFDY

SEQ ID NO: 15 - DNA, 366 bp, synthetic construct
  source 1..366: mol_type = other DNA
gaagttcagctgctggaaagcggcggcggcctggttcagccgggcgggtccctgcgcctgagctgcgcggctagcggctttcgggattccgatgaagatatgggctgggtgcgccaggcacctggtaaaggcctggaatgggtgagctccattggtggttacaacgggtctacctattatgcagatagcgtgaaaggccgctttaccatctcgagagataattcgaaaaacaccctgtatctgcaaatgaacagcctgcgtgcggaagatactgcagtttattattgcgcgagcgaaggttactacacttctgctttcgactactggggccagggtaccctggtgactgtgagcagcgcggccgca

SEQ ID NO: 16 - protein, 122 aa, synthetic construct
EVQLLESGGGLVQPGGSLRLSCAASGFRDSDEDMGWVRQAPGKGLEWVSSIGGYNGSTYYADSVKGRFTISRDNSKNTLYLQMNSLRAEDTAVYYCASEGYYTSAFDYWGQGTLVTVSSAAA

SEQ ID NO: 17 - protein, 10 aa, synthetic construct
GFRDSDEDMG

SEQ ID NO: 18 - protein, 16 aa, synthetic construct
IGGYNGSTYYADSVKG

SEQ ID NO: 19 - protein, 10 aa, synthetic construct
EGYYTSAFDY

SEQ ID NO: 20 - DNA, 366 bp, synthetic construct
  source 1..366: mol_type = other DNA
gaagttcagctgctggaaagcggcggcggcctggttcagccgggcgggtccctgcgcctgagctgcgcggctagcggctttcgggattccgatgaagatatgggctgggtgcgccaggcacctggtaaaggcctggaatgggtgagctccattgacgctgaagacgggtctacctattatgcagatagcgtgaaaggccgctttaccatctcgagagataattcgaaaaacaccctgtatctgcaaatgaacagcctgcgtgcggaagatactgcagtttattattgcgcgagcgactctggtgactggttctctttcgactactggggccagggtaccctggtgactgtgagcagcgcggccgca

SEQ ID NO: 21 - protein, 122 aa, synthetic construct
EVQLLESGGGLVQPGGSLRLSCAASGFRDSDEDMGWVRQAPGKGLEWVSSIDAEDGSTYYADSVKGRFTISRDNSKNTLYLQMNSLRAEDTAVYYCASDSGDWFSFDYWGQGTLVTVSSAAA

SEQ ID NO: 22 - protein, 10 aa, synthetic construct
GFRDSDEDMG

SEQ ID NO: 23 - protein, 16 aa, synthetic construct
IDAEDGSTYYADSVKG

SEQ ID NO: 24 - protein, 10 aa, synthetic construct
DSGDWFSFDY

SEQ ID NO: 25 - DNA, 366 bp, synthetic construct
  source 1..366: mol_type = other DNA
gaagttcagctgctggaaagcggcggcggcctggttcagccgggcgggtccctgcgcctgagctgcgcggctagcggctttcgggattccgatgaagatatgggctgggtgcgccaggcacctggtaaaggcctggaatgggtgagctccattgactctccgaacgggtctacctattatgcagatagcgtgaaaggccgctttaccatctcgagagataattcgaaaaacaccctgtatctgcaaatgaacagcctgcgtgcggaagatactgcagtttattattgcgcgagctacgacggttactactggtggttcgactactggggccagggtaccctggtgactgtgagcagcgcggccgca

SEQ ID NO: 26 - protein, 122 aa, synthetic construct
EVQLLESGGGLVQPGGSLRLSCAASGFRDSDEDMGWVRQAPGKGLEWVSSIDSPNGSTYYADSVKGRFTISRDNSKNTLYLQMNSLRAEDTAVYYCASYDGYYWWFDYWGQGTLVTVSSAAA

SEQ ID NO: 27 - protein, 10 aa, synthetic construct
GFRDSDEDMG

SEQ ID NO: 28 - protein, 16 aa, synthetic construct
IDSPNGSTYYADSVKG

SEQ ID NO: 29 - protein, 10 aa, synthetic construct
YDGYYWWFDY

SEQ ID NO: 30 - DNA, 366 bp, synthetic construct
  source 1..366: mol_type = other DNA
gaagttcagctgctggaaagcggcggcggcctggttcagccgggcgggtccctgcgcctgagctgcgcggctagcggctttcgggattccgatgaagatatgggctgggtgcgccaggcacctggtaaaggcctggaatgggtgagctccattgaaggtccgtctgggtctacctattatgcagatagcgtgaaaggccgctttaccatctcgagagataattcgaaaaacaccctgtatctgcaaatgaacagcctgcgtgcggaagatactgcagtttattattgcgcgagcgttacttggtggactcatccgttcgactactggggccagggtaccctggtgactgtgagcagcgcggccgca

SEQ ID NO: 31 - protein, 122 aa, synthetic construct
EVQLLESGGGLVQPGGSLRLSCAASGFRDSDEDMGWVRQAPGKGLEWVSSIEGPSGSTYYADSVKGRFTISRDNSKNTLYLQMNSLRAEDTAVYYCASVTWWTHPFDYWGQGTLVTVSSAAA

SEQ ID NO: 32 - protein, 10 aa, synthetic construct
GFRDSDEDMG

SEQ ID NO: 33 - protein, 16 aa, synthetic construct
IEGPSGSTYYADSVKG

SEQ ID NO: 34 - protein, 10 aa, synthetic construct
VTWWTHPFDY

SEQ ID NO: 35 - DNA, 366 bp, synthetic construct
  source 1..366: mol_type = other DNA
gaagttcagctgctggaaagcggcggcggcctggttcagccgggcgggtccctgcgcctgagctgcgcggctagcggctttcgggattccgatgaagatatgggctgggtgcgccaggcacctggtaaaggcctggaatgggtgagctccattggtacttacgacgggtctacctattatgcagatagcgtgaaaggccgctttaccatctcgagagataattcgaaaaacaccctgtatctgcaaatgaacagcctgcgtgcggaagatactgcagtttattattgcgcgagctacccgcaggctaacgttgctttcgactactggggccagggtaccctggtgactgtgagcagcgcggccgca

SEQ ID NO: 36 - protein, 122 aa, synthetic construct
EVQLLESGGGLVQPGGSLRLSCAASGFRDSDEDMGWVRQAPGKGLEWVSSIGTYDGSTYYADSVKGRFTISRDNSKNTLYLQMNSLRAEDTAVYYCASYPQANVAFDYWGQGTLVTVSSAAA

SEQ ID NO: 37 - protein, 10 aa, synthetic construct
GFRDSDEDMG

SEQ ID NO: 38 - protein, 16 aa, synthetic construct
IGTYDGSTYYADSVKG

SEQ ID NO: 39 - protein, 10 aa, synthetic construct
YPQANVAFDY

SEQ ID NO: 40 - DNA, 19189 bp, Aspergillus Fumigatus
  source 1..19189: mol_type = genomic DNA
atggcgcagtcaaccgtctcatgctatttacctaactttggtgctacctgtgatggcccgaaacgtcctctttccttgaaactgcgaacagatgctctccaaagctctggattgctgtctgcatggcgcgaagggcaattgacccagttgttgcaggcctcgtgggccctaattctacatcgctacacaggttctgaggatatttgctttggctaccatcagattggtaacgtgtctcaggatctactacagtctttagaagccgtggatcctgccctttgcaaagtatcagtcaaagaaggcatctccctgaaaatgtttttcgaccaattcaagacacataactctcccgacagtcctctcgagatagacagaagctcacgcaaagcctctgaaaatgctcggctttacaacactatcttaatgatacaaatctgtcatgattccaacggaacttcacccgccatcccactaccatccagcctgccaatagcgcttcctcaggaggtgagtactgagagactggtcctttatcttcccgtgcaaccttctgaaacatatttgcagagtcgggttcgacttcatgtcaaagttctacaggacgaggtaaacattttcttcgaatggtggaacaatgacatgcccactgcgcaaatgaaaagcattgctggtgcttttgggcacatcctaacagatctccttgcagcaaacgatacagcagtggacgatcttgacatctttccagaaaatgattggtctcgggtatgcagcttcaattcagtgcttccaaaaaagcacgaacgctgcatccatgaaatgatctatgaccggactcttctccagccagagaatgaggcggtctgctcctgggatggtagccttacctacaaagaattggatttgctttcgtccaaggttgcatatgatcttcaacaaagaggagttggcccagaggtctgcgtggccctttgtttcgagaagtcggtaagtgctgcaggccaaatgatagagcttcccgcgtaccgtcatttcattatctgatttgacatactcggtcgagctctgccctcaatctgcagtgctgatagaaaacatagaaatggtataccgtggcgatgcttgctgtcctgaaagctggcggtgctttcgtacccatggacccaagtcatcctacagcacggttacaatctctcgtggaaggcgtgcaggcgcatattatgctttgctctcgaagtcaaacaggcaagctccaaacagtggccgaaacgctgataccattagatgaggaaactgtcgatggccttccagatcttcccacaagtactttctcctcgacgacagtaaagagctcaaacgccgcatatgttatttttacctccggctctactggacaaccaaaggtgcgtctccaccaattccaatccggtcaccttggtgctaaccccagaacagggcacattgctggagcatagagcatatgtttcaggtgcgctggctcatggaccagtctttggcctcaattcatcaactcgtgttttgcaattcgcatcgcatgcatttgacgcaagtttagtcgatatcctcagctcgttaatattcggctcttgcatctgcatccctagcgaggaagcacgcctgaatgatattgctggcgtgatcaacgagatgaaagtgaatcacgcatcacttacaccatcatttgttggattcctcgatcctgcagctgttccagggttagaaagccttgttctagctggtgaagcaatgtctccgcaacatttggcgacatggtctcatatcaagcttgtgaacggatacggcccaacagagagttctgttgcggctgccctcaatcctaatatgtcttcttcatcagattgcagggatattgggttgccagtcggagtgcgcttctgggtcgtgaaccctgccaaccatgaccagcttgtgcccgttggttgccccggtgagctggtactggaaggaccgacgcttgcgcgatgttacatcaataacccacagaagacgagcgattcatttattttcaacccttgttggacgaagcgagatcctaacggtggctcagaccgaaggttttacaagaccggtgatttggtaaggtacaactccgaatccggatcattaacgtatataggacggaaagacgcacaagtcaagttccatggccagagagttgagcttggagagattgagagtcagctgagcgcagacacagacatcaagcactgcacagtgttgcttcccaagtctgggttcgcacaaggaaaacttgtgactgtcgtgtcactgtccgctggccctggacaggcactggaggccgatgcagtgccccttaagcttatcgagcatcgcgagaagcttcgatatgtcaagagtattcaggaacgattatcaattaggctgccgacatacatggtgcccggagtatggctatgtgtcgaggctttgccgatgctcgtgtctggtaaactggaccggaaatctattgcaacctgggttgcgagcatgtcagaagacccagaagtacatgcaactgaggccgccaacgctcgtgccgctaactcgacggaggatcaactggtgtcgatttggagtcgtgtgctcaatgtacccaaggatcgcatcagcgtcgacgaaagctttttgagtctaggaggagattccattgcagcgataacctgtgttggccattgcaagaagcaaggcattggtctcactgtgcaggagatacttcgcagcaaatccatcagagaactagccactcgtgtcaagggagtgactcaaccggtagcttatcacgaaatgattgaggaaccttttgggctttcaccaatacagaaactgcatttcatggtccgtaaggaaggacagggctatttcaatcaaagtgtagtcactcgcatcgaccgtcagatcaacgaccaagatatgcgccgcgcggtcgaggcagtggtcatgcgccactccatgctccggagccggctagtcgatcccagtactggcaattctctacagctgcgcattactgaggacgtcgctggctcgtaccgctggcgaacacattatatgaccgcgcagaacgagattgagaatgctattgcggagagccagttgtgcatcaatgccttcgtgggccctgtttttgcagttgacttctgctatgttgacgaggactcgcacaacctcctgtccctggtggctcatcatctggtcgttgacatagtctcgtggcgtatcattctggaggacttggaagatttcctgctaaaccctcaaggctttgttttacagaatagctcccttccatttcagacatggtgccgcctgcaagacgagcaatgtgagtctgtggctttcgagaatgatgtgcaattagaggatcttcctgctccagaccttgcctactggggaatggagcatcgtcagatgacgtatggcgacgtgatctgtgagacattcgagcttgatcccggcagcactcagtcgattttgcttgaatgtcatcagagtctacgaacagagcctgttgacctgttcctcgctgctcttgtccactcatttggacagactttcccagagcgtaccctgcccgtgatctacaatgaaggtcatggcagagaagtgtgggattctagtttagacatatcccggactgtgggctggtttacgaccctttacccaatcttcgtacaggaaatcgtctccgaggatccggcccgaactgttgcccgtgtcaaagatctgcgtcggcaggtgtctgataatggccgacagaagttcgcaagtcgcatgttcacaggcaaaggacagcagacatgccgacaccactaccccctggaaatgactttcaactacgtggggcagcatcgcgatctgcagaagcaggacggtcttttccagcttatgggacacatggctggtgaggctggccagggtggaggtgcagcagattttggcgaagagactccacgattcgcacttctggaaatttcagcattggtagtgcaaggccaactccgttttacgttctctttcaaccgattcatgcggcatcaaagtggcattcacgcctggatatcacgttgccatcagctgcttgcttctctcggccagaagctacagtctctggctcctcagcccacactgagtgatttcccaatgctctctctcacctacgaggaactcgacaagatggtatcagcgaagctaccaatcgccggtattacctcccttgatcttgtcgaggacgtgtatccttgctcgcgcatgcaacagggtattttgctcagccagtcccgaaacacctcggtctatgctgtgcatgatactttcgaagtgaaaggcgtcggaatcaaacctagtgtggatcggctgatcatcgcttggcagaaggttgtttcgcgtcatgcaatgctaagaacagttttcttggagaacctgacaagccaggaccttttttgccaagtcgttctcaaggaatacaacccagctcccgcactgctgagctgctccgaagaacgtgatgttttgccaacctttgataatcaacagcccgtgaactatcgtgacccccgtcccgctcatcgttttagtatctgcgagacagcaaatggcaaactgttttgccgacttgaaataagccatgccgccatggatgggacttcaatctctctgatcgttcgagatctgcaatcggcgtacagtggtcagcttcaggaagaccgcaaacccatgttcaaggattacatgcggtacttgcagagctgctctcatagtgctggcctgaattactggctcccctatttatccgatgtcaagccgtgccatttcccggtgctcaatgatggaagaccttcaaacaagcggctgcaagtaattcgtctcgacttctcgtctttgaaagaacttcaggcactatgtgagagctgtggcttgacgctttctacggcgtttagtacggcttggggacttacgctccgctcgttttgcggttccaacgaagtctgcttcagctacatggcttctttgcgggatgtctcggttgatgaaattggttcggttgtcggaccggtcattaatcttctggcttgtcgaatgaaggtcacagaggatgtctgtcttgaagacgtcctgcatcaagtgcaaaacgattacatggaaagtctgccttacagacacacttccctcatcgatatacagcatgccctgaaactctcagacaccattttgctcaactcaggcatctcctaccggaaacttcccccgaagaccctcagcaacagggatgagatgcgacttgtggaggtgggaaaaatccatgatcccgcggaattcccggtgtatgtcaacatcgaggccacggatgatgttgcatacatcgacctcaactactggacgacctctctgtctgaaggccaggcacagaatgttgccagcacctttttacagtccctcgagaatatcattcaccatcacgacgagaaaatttgccgcttggatcaattgagcgcacagaacaaacaacaaatcgctgtctggaacaacactcttcccaaggctgttgagaagtgtatccatgagattctcgaggacaaggtcaagcagtgcccggaagctgtcgctattgcagcttgggatggaaacctcacttatgccaagctcaatgagttgtcctcgcttcttgcgttctatctgacaaaactgggcgtgggacctggtctccttgtgccaattgacttggacaaatcctcgtggcaaattgttgctatcttagcggttctgagagccggtggtatttgccttccagtcgacgcagctcaaccatatgagttcattgaaaagctgttgattgacaaagacattcaggtcgctcttgcgtccccaaacaaagcgcagcttctggaacgcacaattccttatgtcgtccccgtgggaaggtcactttttgactacctccctcgttttgatgatatgccgcacgtctcacataaggcaatggaccatgcttacgttgtgttcactggcggtagtgtcaaggaaccaaaaggtgttatgctccagcatcttacagttttgacacgggctgagaatttcgcatcggctctggaactgaataaagcgaccaaggtgtaccagtccgcgacgtatacatctgacatgttcctcaatgtgctttttggtacaatgatgcgaggtggttgtgtttgtattccagccaacgatggattcaacaacctcccacgatcgatcaatgcctcccgagcaaacacagttgttatgactccgtcactcgcatctctgttgcagccatctgaggtccctgaggtgcagctactggcactatatggtgagatcctgacaaaccaggtcagaactatttggtcagaaaaagttcgcacgcattctttgtatggagcagccgagtgctcttcaagttgcattcatgcttctgactgccaaaccttgggtgagacaaggaaccttggacttgcggctgggtgcataacttggcttgttaatctttcggaccacgaccttctggttccgattggtagtgtcggtgaagttgtcattgagggtcccgtcatcgcttctggttacttgttgcataacgggcatgtcaagggaggcttcattgaaaatcctgtttggagaatgaactttgagcgtgacgaacccagcaatgacctcgagaaacgtgacgaaagctcaactctgacccgacggatgttcaagaccggggacttggcccgctacaattcggatggcatattggtctacatgggaagaaaggaaagacagacccagagattgcaagcggatatatgggacgtccaacagtgcatcgacacgttttcgttacctggccatccttgcgttgtggagccgattcgctgcttagatgacgatgaatccgtggagcacctggctgtctttgtccaattcgcctcaactcacttggagaaggctgatggccagagatctgtaatcggacaaccctcctcgcaattttttgattctgtcaccaaaatgcacacctatctcctgagtgtcctgcctgtgactcaagtcccgaggttgtacattcctgttccaagcatgcccttaacatcgactggtctgcttgaccgttggttcctgagaaatgaggctcagaacctccctgcgcagacgcgcatcgaatttgacctgaaaagctttcatgatttttggcgggttgaattagcccatccgaagccttctccttcccagctactcccttcatcgacgagtgctacccataggagctctggtaccagcacgtacgaatggaacggccacattgaatggcgtgatatcagcaaattggaaagtgcaagtctcgaggccgcgcttcttgccgcatgggcactcactatatccggctacactcgatcggacgatgtcatctttggagaattgcttcttgagcaggattcgtcagggctggattctacttccgataaagcggcgccagtggtcgttcctagaagattacaaatcgctgaagatatgagcattgcggaacttatgagaaagacccaagagcgcctagtagcggcttctcccttccaaagagctggacttcaacgcattagaaacgtcagcgcggatacttcgcgtgcgtgcagctttaataaccttttctgctttacaagatttgactgcaaggtgcagactcttgctctcgcatatcctctcgggatcttttgtatagtcgcgaactcggagctccagttgagcgcttgctacgacgaacagattttgtctgctccccaggttgagcggattttagtacaattcgcacgctacgttgaatacttgaaggcggatctccgctctcaagagacgatcggtgacatggctctccgcaagaatcagacttcttacttgtctagccctgagactgtctactggaggaagtaccttgctgatgtcgagtcctgtgtattcccttctttgaacccagacggtgaaagaagtggattcagctcggcaaaactggcgattgaaaatctggctgatttacgccgcctttgccagaagattgaagtaaccgaggacattgtgctacagctcgtttggggattggtcctgcgatgctataccggttcagaagaagtctgctatggttactaccaggcatcaccacaacccgaggggtcgaagtacctgaaagtactcccaagcaggtttcttcttaaggacgactcagaccttgagtcaattgcacagcagagaaagagtgagctggatgaagccatggagcaccccatctcccagatagaactgcagcttgaacttggctttgactggtattcgctcttgaacactgtctttaaattcgacagatttgcggagctccccaatgacaataacagtacattagacctgctcaacgacaccgagaaagggatctggactattgttgtcaaccctagattttcatttgtatcggcagatatcctcttcgaataccggacagacgccttgtcagaagccaacatcacgagtgttgttgattgcttccagcacattctcgaagagattatcaacaatgatcctgtcggccataagataggggacatcaatttcttcggcgaacgtgcttgtcaacaagtacgagagtggaacgctgcgctaccagagagacctgatagatgcgcacatgagataattgaacaacaggttctgagccaccctacctcgcctgcaatctgctcatgggacggagaatttacttacgaacaactcgaccgtctatcaaccaaattggcaaagcatcttgtttgcctgggtgtcaagcccgaaatcttcgttggattgtgctttgagaagtccgcttgggcagtcatcgcacaagttgcggtcctgaaagccggaggggcatttgcatctctggatcccagtcacccggacgcaagacttaggggcttggtcgacgatattggcgctcatatctttctttgctctgccaaatatctcgacaaggctcgccagatatcccgcgccgcctatatcgtgagtgaagaaacactcgctgaactgccagacgtatcgtctactgcgtcgatgactcggcccagcatccacaacgcagcctacgctatttttacctccggaacaacaggcaagccaaaagtgactgttcttgagcacattgctctcagcgtgtcttcaccagctttcgctcgtagcatgggaatggacacgactactcgtgctctgcagttctccagttatacttttgatgtgagcataaaggagattatcattgtcctcatgacgggaggttgtgtttgcgttcccagtgatgaagagcgcatgaacgacctgtccggtgccatcaggagactgaacgccaacttcatcagttgtcctccgtcagtttcaaacacgatccaacccgagagtgtcccatcagttaaaactgttgtgatgggaggcgaaaagatgactgcgagccatatcgaccgttggggcgataggtttgtcatcaacgcgtacgggccctcggagtcgacagttatggcaacgatgagcgtcaaagtggatgaagcaggagtacgcgtcaacaacgattgcaactccatcggtgctgcgatttgcggccgcacttgggtggttgaccctaacaactatcagcgtcttctgcctatcggggctgtgggtgagctggtacttgagggctgcaacgttgcccgtggttacctcaacaatgaccagaagacaaaagagtcatttatcagcgaccctgcatggacgaaggccccaggtttgaaggaactattcaagaggaaggagcgcatgtaccggactggtgatcttgtccgctataaccccgacggcacaatttgcttcatctcgcgcaaggacacccagatcaagttcaacggccagcgaattgaactggaagaaatcgaacaacaatgcatcagttttctttctggaggcactcaggtggccgtcgaggttgtggagcccgaatcgaaggcagttgcaagatccatcgcagctttctttaccgttgacaatcaatctggtcaagacagacctgatttggaatctcaattgctcgttcctatgtcggaagcaacacgcgaaaaggtccagaagcttagggaggccttgattaaggctctgccaccaattatgataccaaggctcttcttcccagtctcccacctgcctttctcaaactccggaaagctcgatcgcaagaagctccgggcaaccgttgagactctacccaaagatcagctcaaatcttacgctacactgacggctggctccagacaagcgtctgatgagggagttgaggggacactcagaagcctctgggaagaagctctgggtcttgcgtcaggttcggtcagtgctgaagacagctttttcagcttaggtggtgattctttctccgccatgaagctcgttggagccgccaattcccagggaatctctctcacatttgccgacgtttacgaggatccagtctttatgaacatggccaagcgctgtggcatgttacagggaaggtccggccgacagacagtgactccgtttagcctgctgcccgcctcagtcgaccgtgaacagctcttggaggaagtagcggaacaatgtggcgtgcctcgagcatccattgtcgatctctacccatgcagccctgtccaggagggtctcctcactttgtcagtcaagcagaacggtgcttatattgctcagccaatcttccggctctcagagggaatagatctcgacatgttcaaagctgcttggcaacaggttgtggatgagctggatatcttgcggactcgtattgtgcatactgagtctctgaactttttgcaggctgttattgacaaagaagagatctcttgggcttcagcaaccaccctggatgaattgacagccgagagccccgagctgccgcgacacaatggcgggcggctgactggttatgcgattgccgcgtcgcagactggccggtacttctgttggacaattcatcacgcgctgtacgacggatggagtatcccgctcgttctacgaagagtggaggaggtttacacgaattccacagctagcgctcgaacagttccctacagcttatttataaactatctgctggaacggagcatggcagactctgacgagtattggaagtctcaactggccaacctatcctgttctccattcccacaaagcaggaatcccctgcctgactctgttcgagtaggaaataggcatcacagcagcatgaagatctctcgagccgccagcggagtggatcttactatccccgagcttattcgtgctgcgtgggcgatcgtcgtctctgcccatactggatccagtgatgtatgcttcggcgagacgctcatgggaagaaatatcgacctcgctggtgttacagatattgctggacccgttcttaccactgtccctacccgcattcaagttgacaatgagctgccaattacccagtatctggagaatatgcaccatctgacgaccacaatgcttcctcatcagcattcaggactgcagcaaattcgaaaactcaacagtgacacagcatcggcttgtgaatttcaaaatctgcttgtcatccagaccggagagggccagttgaacaaggccttgtgggtggctgaacccatccaaaccagtggagacttcttcactcatccacttgtcgtcgaatgcaaggtcgatacttctgaggtgtcgattacgatgcaccatgacgaaatcgtactgaacagctggcagactgagaagctcataggtcaattcagcttcgtgctcgagcagctgctctccattaacaagggggagacaaggaagttgagcgagctcgagattttcagtccacttgatagcaaggaagtcgcactatggaacaagaggcaccctgaggccgttgagaaatgtgctcatgatatcatttccgagagatgctctacccaccccgacgctccagctgtctgcgcttgggatggcgaggtctcgtacaaagagatgtacacacttgcctcgtcttttgcgtcctatctagcttgtcgtggcgttggcccagagactctggtgccaatttgcctggataagtctctgtgggcaatcatcacaattctgggaatcttgattgctggtggtgcctatgtaccactcgatcctgctcatccaacatctagacatgaagaaatcctcactgaagtggatgctcgtattctgatatgctcccctcaataccagagccgttactctagcattgtcaaaacgataattcccgtcagcaaggagacgatcagagcttattttgccctcaattaccaggcgaaaggactcaggcgtgtcacgccgttcaatatggcctatgcaatctttacctcaggaagtactggtcgcgccaagggcattatcatcgaccaccgcgcacttgcaagcagtgccatggcattcgggccgattgtgcacttgaatgagacttcccgtgcattccaatttgcgtcccttacctttgatgcagctgtcatggaaattctagctactctgatgcatggaggatgcatttgtatcccaagtgaggatgagcgcctgaatgatgtcgccggcgccattcgacgtatgaatgtcacatggacttttctgactccgtcgatagctagcattatcgagccatcaaccgttccctcactggaagtgcttgcctgtggaggagaaaaattgtcacgcgaagtcgtgacaaaatgggcgcatcgcgttaaactcatcaatggatatggacccacagagacaaccatcttcgctgtcttgaataacgtgtccccgactaccgatcctgcttgcattggttacggaataccatgcactttgacctgggttgtcgatcccgaaaaccacgatcgacttaccccattgggagccatcggagaacttgccttggaaggtccagctctggcaagggagtatttgaagaaccccaagaagactgccgaggcattcgtggatgagccagcgtggatgaagcatttccagtcgaccctgccgtctccgcgaagaatatacaagactggagaccttgtccggtataaccctgacggatcggtggaatacatcagccgcaaagactaccaagtcaagcttcatggccagcgcatggagctcggtgaaattgagcatcggttacatgaggacgaccgcgtccgtcatgccattgtcatccttcccaaggagggtcttcttaaggggcgtctggtcacgattctctccctgaactctctgaaatcaggttcgagcatcatctccgataatgcatgtgagcttatcagtcgtgaggatctggcgagagtcgcgtattctgagctcatcacgattcagaagaatctcgaagcccagctgccgatttacatggttcctcagacctgggctgttatcaagaagttaccaatgctcgtttctggaaagctggatagaaagaagattactcactggattgagaatatcgacgagccaacatatgacaggatcatgcaagactacgacaacattaagagaggtcatgttgaggacagcgtcaatgaagataagagcactgccgccaagatcttgcaagacatctacgcccaagtacttaacctcccttcaaacaaggtagaccccaagcggtcgtttgtgagcctaggtaagtggatcattgtccgtctcatgaagaagagaggaaaagaggctgactggtaaacacaggcggtgatagtattactggaatggctgtcatttcccgagctcgcaaacaagggctcaacctgacacttcacaagatccttcaaagcaagtctatcgttgagctgattcaagcagcggaagttgagacctcgagcattcaagtcgaggagaaggccaacaaatacttttccctttctcctatccaaaatttgtacttcaaatcagcacgcaccttcaaggaaacaggtcgcttcaatcagggtatgactgtgcgcgttactcgaaaggttgagccaaacgttgtcaaggatgcactcaaggcagttgccagccagcattccatgcttagggcaagattcagcagatctgcaaacggaaaatggcagcaacggattaccaatgtacgtgcaaaccaactcactgacgtggtcttaaggagctgaccgatcacacaggatatcgagtcatctgttcgtgttggtattcactcagtcatgagctcccatgagatgctgggcaagattgccaatactcagtcgtctctggatatcgagaatgggccgatcattgcggccgatctgttcactgttaacggcgaacaagtccttttcctggtcgccaatcacctgtgtgtcgatatggtttcatggcgcattatccttcaagacatacaggaggtcatcgaagctggctcgctgtcttctgagaaacccttttcgttccagagctggtgcgaactgcaactcgaaaacagcagatcagaagcagacaaggccaagcttccgtttgccattgagcctccgaaccttagctactggggcatggagagcgtgcccaatcattacggacaaatcaggatggaatccttcgttgtgggcgaagacactacttcgtttattctgggtgactgccacgagatgctgcgcacagaaacaatcgatatcctgcttgctgcggttgcgcagtcattccgtcgggtctttacagatcgcagaatgccaaccatttataacgagggtcacggtcgtgaatcgtgggattcgaaccttgatctctcacggactgttgggtggttcaccacgttgtgtccactacaggtggacgaatgctcaggtaaatatcttctcgaaaattctagagcgacacgctaatattagggtcagatttcgtcgacacgacaaaacgtgtgaaggacctaaggcggaagatcaaagacaacggccgatcttacttcgctcgcagccttctccaggccaacaataccgagccatcggacttcccagttccgttggaaatcgttttcaactatcttggtcgtttgcagcaactggagcgcgacgactcgcttttcaagcactatggggaggcgttcgacgaggagaagttcagattagccggagatatgggatcagataccccccggtttgccctcctggagatttccgcacttgtcgtcaacgacaagctgcaggtttcattcacgtacaaccggcaaatgcagcgtgaaagccagatcttccaatggatttcagagtgtagaagagtacttgagattgatgtgcttcgcttcaaggacaccgtgccggaacccaccctcagcgatttccccttgctccctataacgtatgatgggctcaagaagctcacgagcacaacgctgccaagggcaggggtcaagaccttcagccaagtagaggatatctatccttgctcatcagtgcaagagggtattctcctgagccagctccgggatcccagtgcgtacatgttccatgtgatcttcgaagtgcgctctcctggaggaagcggcaaggtcgatccaaacatgcttcgaagcgcatggtcagcagttatcaatcgacacccaatcctgagaaccctgttcatcgatagtaactacgcaaacggtaccttcgatcagctggtgctcaggaaagtcgatgaaggagccatcattctccattgcaatgattccgacgcacttgtcaagctcgatacaatcaaactcagtgaaatcaacgctggacgatgcccaaagctactacatcaattgacagtctgcagcactgattcgggacgagtgcttatcaaattggagatgaatcatgctatcatagatggcggatcagttgacctgcttttgagggacctagcaatggcgtacaagcatcagctgccagaaggatccggtccgcacttcagtgactacatcaaattcgtccgcgggaaatctcaaagccaagctttatctcactggagacaatacttgtccgatgcacatccctgccatctcacgttctccgaaggaacaggtggtagtcgtcaattaggatcagtgatggtgccgttcagccggtattctgagctgcaacaattctgcgagaagaactccgtgacactcgcaaatcttactcttgctgcatgggcgatcgtcttgcaaagctttacgggctcgaatgatgtttgctttggttacccttctgctggtcgtgacgccccagttcccgggatccaagacgcagtcggaatattcctcaacatgctctgctgccgagtcaggttctcaccagggaagactctgttggaggtttcaaagacagtgcaagatgactatatcaagaaccttccataccaggattgttccctcgcaagtattcagcatgaacttgggcaaaaggagctgtttaacacgacaatctcgattcaaaaccaccacgctgtctcagaagaatctggaaatgacttgctctcttttgatgtacagacggcgcatgacccgacagaggtaaggtttacccaaactatttttatatttgatagaagctgatttctgcaatagtatcctgtcaccgtgaacgtcgagactgccaagggccacgaagggatccttctgcgatactggaccgatgctgtgccagaagacaaagcaaacaatcttgcaaatgccattgcccatattttcagctgctttattgacaagccgtctcagctggtctcagagctggacctccgtgggggacaattgatgaagggcggtcagttcattgacagcaagtcccttcaggaacttatcgatcgccgtgtcactgaaataatcagtcaaatgctcaaggaaggaactctggccataccggcagctgacaatggaaagggccagttcaagggcacgacccgggcccaacctgctaaagttacaataaaggcgagaggtatgcatcgtgaaagagatctgtccgactcgacggcaaccctcaccgaggatcactcaaaattgatggaaccagaaaagagattatggaaaatttggtgctcagcattgggcctggcatctgacactatccaacgccaggcgagcttcttcaaattgggtggtgatagtataactgccatgaagatggtcagcgctgcgcgcgaagacgggctgacactgaccatggccgatgttttcaacaatcctgtctttgaggacatgctggcggccatcagtgcttccaacagttcgtctgctttggaacctgactctccggccgacagcaacaatgagaaacctgctgaacctcctaggttggttgagttagagcggaacccacccccaccggagatttcactcttgaaaaccgttcctttgaacgacagtgctctgcaagatggaatttgcccgaagattggcgtgtttaaaggaggaattgccgatgttttgcctgttacggacttccaagccatgtcgataacggctacattgttcaagtcccgctggatgttgaactacttctttttcgacggacgaggatctctggatctgagaaggttgagagagagcctcttgcgcgttgtcgatgctttcgacattcttcgaacagtctttgtctgtttcaatggccaatttttccaagtggtgctcagaaagatacggccaaacatctttgttcatgaaactgagaagaacttggatgagtatactgagtacctacaacagcaagatcgcgagcaggaggcgcggcaaggcgaacagtatgtgaagttctacatcgtgaagaagaagggcagcaaccaccaccggatcttgattagaatgtcgcacgctcaattcgacggactatgcctgcctaccatcatgtcagccattaaattgggatacgagggcagtactctgccccctgctccttcctttgcgaactacatgcgcatgctctcgggagcaatcactccagaccactatcagcattggacaaacctgttgaagggatcgaaaatgacgcaggtaattcgaaggaccgaggagaatacctatcgttatattggtgcttttagagagcaaagaaagacactggagatccagccctcggtcctggaaaatgtcaccattgctaccgtcatgcaagcagcatgggcggttacgctggccaagctgtccgctcagtcagatgttgtcttcggtctcacgatcagcggcagaaatacttcaatcccaggcattgaaaacactgtcggtccatgcctgaatgtgattccgattcgagtcacatttaaagagggctggactggagttgatctgttccgctacctacaagatcagcagatagccaatatgcccttcgaggccttgggtttcagggagatcatcagacgctgtactgactggcccagttcgacatactttaccacctcggtgttccaccagaatgtcgagtacgagggccagatgcagttagacaacactacatacagaatgggaggcgcgggtgtcgtcgacaacttcactgacatgacattgttctccaagtcatccagcgacggcaaactgggcgtctctctgggctattcagacaaaggccccatcggccccaaatttgctgccaaggtcctaagcatggtctgcgacaccgtgcaaagcctgttggccaactcgcgtgttgggcttccctccccatctatgctgcgctcattaccttgtcaatccgtccacgacgtccccggaatgaccgatgagatgtttttgtccacacatctgaaggaccggagtatctcggatatcctcgtccactcggacgtcgtgacgcaagcctggcagcaagtcctccctcgcaataatgctgacgagcctcaatcgtcattccagctcgactcgtccttctttgaactgggaggtgatgttttcgacatggctcaggtcgtctggcttctcgagcaggaaggactgcaggttcacatcgaggacctccttgaacacccgaccttccttgggcagatggcagtcttgacactgcacaactcacggacgagcgattccatggaagaaattgtgcccgtcgaggagatccccctacctgcagcgagaacaggcacttcgagttctctaggcaaagccttgactctggcgaagaaggtcacaaggtggagcgcactgtcggctcgagggtga

SEQ ID NO: 41 - protein, 6269 aa, Aspergillus Fumigatus
MAQSTVSCYLPNFGATCDGPKRPLSLKLRTDALQSSGLLSAWREGQLTQLLQASWALILHRYTGSEDICFGYHQIGNVSQDLLQSLEAVDPALCKVSVKEGISLKMFFDQFKTHNSPDSPLEIDRSSRKASENARLYNTILMIQICHDSNGTSPAIPLPSSLPIALPQEVSTERLVLYLPVQPSETYLQSRVRLHVKVLQDEVNIFFEWWNNDMPTAQMKSIAGAFGHILTDLLAANDTAVDDLDIFPENDWSRVCSFNSVLPKKHERCIHEMIYDRTLLQPENEAVCSWDGSLTYKELDLLSSKVAYDLQQRGVGPEVCVALCFEKSKWYTVAMLAVLKAGGAFVPMDPSHPTARLQSLVEGVQAHIMLCSRSQTGKLQTVAETLIPLDEETVDGLPDLPTSTFSSTTVKSSNAAYVIFTSGSTGQPKGTLLEHRAYVSGALAHGPVFGLNSSTRVLQFASHAFDASLVDILSSLIFGSCICIPSEEARLNDIAGVINEMKVNHASLTPSFVGFLDPAAVPGLESLVLAGEAMSPQHLATWSHIKLVNGYGPTESSVAAALNPNMSSSSDCRDIGLPVGVRFWVVNPANHDQLVPVGCPGELVLEGPTLARCYINNPQKTSDSFIFNPCWTKRDPNGGSDRRFYKTGDLVRYNSESGSLTYIGRKDAQVKFHGQRVELGEIESQLSADTDIKHCTVLLPKSGFAQGKLVTVVSLSAGPGQALEADAVPLKLIEHREKLRYVKSIQERLSIRLPTYMVPGVWLCVEALPMLVSGKLDRKSIATWVASMSEDPEVHATEAANARAANSTEDQLVSIWSRVLNVPKDRISVDESFLSLGGDSIAAITCVGHCKKQGIGLTVQEILRSKSIRELATRVKGVTQPVAYHEMIEEPFGLSPIQKLHFMVRKEGQGYFNQSVVTRIDRQINDQDMRRAVEAVVMRHSMLRSRLVDPSTGNSLQLRITEDVAGSYRWRTHYMTAQNEIENAIAESQLCINAFVGPVFAVDFCYVDEDSHNLLSLVAHHLVVDIVSWRIILEDLEDFLLNPQGFVLQNSSLPFQTWCRLQDEQCESVAFENDVQLEDLPAPDLAYWGMEHRQMTYGDVICETFELDPGSTQSILLECHQSLRTEPVDLFLAALVHSFGQTFPERTLPVIYNEGHGREVWDSSLDISRTVGWFTTLYPIFVQEIVSEDPARTVARVKDLRRQVSDNGRQKFASRMFTGKGQQTCRHHYPLEMTFNYVGQHRDLQKQDGLFQLMGHMAGEAGQGGGAADFGEETPRFALLEISALVVQGQLRFTFSFNRFMRHQSGIHAWISRCHQLLASLGQKLQSLAPQPTLSDFPMLSLTYEELDKMVSAKLPIAGITSLDLVEDVYPCSRMQQGILLSQSRNTSVYAVHDTFEVKGVGIKPSVDRLIIAWQKVVSRHAMLRTVFLENLTSQDLFCQVVLKEYNPAPALLSCSEERDVLPTFDNQQPVNYRDPRPAHRFSICETANGKLFCRLEISHAAMDGTSISLIVRDLQSAYSGQLQEDRKPMFKDYMRYLQSCSHSAGLNYWLPYLSDVKPCHFPVLNDGRPSNKRLQVIRLDFSSLKELQALCESCGLTLSTAFSTAWGLTLRSFCGSNEVCFSYMASLRDVSVDEIGSVVGPVINLLACRMKVTEDVCLEDVLHQVQNDYMESLPYRHTSLIDIQHALKLSDTILLNSGISYRKLPPKTLSNRDEMRLVEVGKIHDPAEFPVYVNIEATDDVAYIDLNYWTTSLSEGQAQNVASTFLQSLENIIHHHDEKICRLDQLSAQNKQQIAVWNNTLPKAVEKCIHEILEDKVKQCPEAVAIAAWDGNLTYAKLNELSSLLAFYLTKLGVGPGLLVPIDLDKSSWQIVAILAVLRAGGICLPVDAAQPYEFIEKLLIDKDIQVALASPNKAQLLERTIPYVVPVGRSLFDYLPRFDDMPHVSHKAMDHAYVVFTGGSVKEPKGVMLQHLTVLTRAENFASALELNKATKVYQSATYTSDMFLNVLFGTMMRGGCVCIPANDGFNNLPRSINASRANTVVMTPSLASLLQPSEVPEVQLLALYGEILTNQVRTIWSEKVRTHSLYGAAECSSSCIHASDCQTLGETRNLGLAAGCITWLVNLSDHDLLVPIGSVGEVVIEGPVIASGYLLHNGHVKGGFIENPVWRMNFERDEPSNDLEKRDESSTLTRRMFKTGDLARYNSDGILVYMGRKERQTQRLQADIWDVQQCIDTFSLPGHPCVVEPIRCLDDDESVEHLAVFVQFASTHLEKADGQRSVIGQPSSQFFDSVTKMHTYLLSVLPVTQVPRLYIPVPSMPLTSTGLLDRWFLRNEAQNLPAQTRIEFDLKSFHDFWRVELAHPKPSPSQLLPSSTSATHRSSGTSTYEWNGHIEWRDISKLESASLEAALLAAWALTISGYTRSDDVIFGELLLEQDSSGLDSTSDKAAPVVVPRRLQIAEDMSIAELMRKTQERLVAASPFQRAGLQRIRNVSADTSRACSFNNLFCFTRFDCKVQTLALAYPLGIFCIVANSELQLSACYDEQILSAPQVERILVQFARYVEYLKADLRSQETIGDMALRKNQTSYLSSPETVYWRKYLADVESCVFPSLNPDGERSGFSSAKLAIENLADLRRLCQKIEVTEDIVLQLVWGLVLRCYTGSEEVCYGYYQASPQPEGSKYLKVLPSRFLLKDDSDLESIAQQRKSELDEAMEHPISQIELQLELGFDWYSLLNTVFKFDRFAELPNDNNSTLDLLNDTEKGIWTIVVNPRFSFVSADILFEYRTDALSEANITSVVDCFQHILEEIINNDPVGHKIGDINFFGERACQQVREWNAALPERPDRCAHEIIEQQVLSHPTSPAICSWDGEFTYEQLDRLSTKLAKHLVCLGVKPEIFVGLCFEKSAWAVIAQVAVLKAGGAFASLDPSHPDARLRGLVDDIGAHIFLCSAKYLDKARQISRAAYIVSEETLAELPDVSSTASMTRPSIHNAAYAIFTSGTTGKPKVTVLEHIALSVSSPAFARSMGMDTTTRALQFSSYTFDVSIKEIIIVLMTGGCVCVPSDEERMNDLSGAIRRLNANFISCPPSVSNTIQPESVPSVKTVVMGGEKMTASHIDRWGDRFVINAYGPSESTVMATMSVKVDEAGVRVNNDCNSIGAAICGRTWVVDPNNYQRLLPIGAVGELVLEGCNVARGYLNNDQKTKESFISDPAWTKAPGLKELFKRKERMYRTGDLVRYNPDGTICFISRKDTQIKFNGQRIELEEIEQQCISFLSGGTQVAVEVVEPESKAVARSIAAFFTVDNQSGQDRPDLESQLLVPMSEATREKVQKLREALIKALPPIMIPRLFFPVSHLPFSNSGKLDRKKLRATVETLPKDQLKSYATLTAGSRQASDEGVEGTLRSLWEEALGLASGSVSAEDSFFSLGGDSFSAMKLVGAANSQGISLTFADVYEDPVFMNMAKRCGMLQGRSGRQTVTPFSLLPASVDREQLLEEVAEQCGVPRASIVDLYPCSPVQEGLLTLSVKQNGAYIAQPIFRLSEGIDLDMFKAAWQQVVDELDILRTRIVHTESLNFLQAVIDKEEISWASATTLDELTAESPELPRHNGGRLTGYAIAASQTGRYFCWTIHHALYDGWSIPLVLRRVEEVYTNSTASARTVPYSLFINYLLERSMADSDEYWKSQLANLSCSPFPQSRNPLPDSVRVGNRHHSSMKISRAASGVDLTIPELIRAAWAIVVSAHTGSSDVCFGETLMGRNIDLAGVTDIAGPVLTTVPTRIQVDNELPITQYLENMHHLTTTMLPHQHSGLQQIRKLNSDTASACEFQNLLVIQTGEGQLNKALWVAEPIQTSGDFFTHPLVVECKVDTSEVSITMHHDEIVLNSWQTEKLIGQFSFVLEQLLSINKGETRKLSELEIFSPLDSKEVALWNKRHPEAVEKCAHDIISERCSTHPDAPAVCAWDGEVSYKEMYTLASSFASYLACRGVGPETLVPICLDKSLWAIITILGILIAGGAYVPLDPAHPTSRHEEILTEVDARILICSPQYQSRYSSIVKTIIPVSKETIRAYFALNYQAKGLRRVTPFNMAYAIFTSGSTGRAKGIIIDHRALASSAMAFGPIVHLNETSRAFQFASLTFDAAVMEILATLMHGGCICIPSEDERLNDVAGAIRRMNVTWTFLTPSIASIIEPSTVPSLEVLACGGEKLSREVVTKWAHRVKLINGYGPTETTIFAVLNNVSPTTDPACIGYGIPCTLTWVVDPENHDRLTPLGAIGELALEGPALAREYLKNPKKTAEAFVDEPAWMKHFQSTLPSPRRIYKTGDLVRYNPDGSVEYISRKDYQVKLHGQRMELGEIEHRLHEDDRVRHAIVILPKEGLLKGRLVTILSLNSLKSGSSIISDNACELISREDLARVAYSELITIQKNLEAQLPIYMVPQTWAVIKKLPMLVSGKLDRKKITHWIENIDEPTYDRIMQDYDNIKRGHVEDSVNEDKSTAAKILQDIYAQVLNLPSNKVDPKRSFVSLGGDSITGMAVISRARKQGLNLTLHKILQSKSIVELIQAAEVETSSIQVEEKANKYFSLSPIQNLYFKSARTFKETGRFNQGMTVRVTRKVEPNVVKDALKAVASQHSMLRARFSRSANGKWQQRITNDIESSVRVGIHSVMSSHEMLGKIANTQSSLDIENGPIIAADLFTVNGEQVLFLVANHLCVDMVSWRIILQDIQEVIEAGSLSSEKPFSFQSWCELQLENSRSEADKAKLPFAIEPPNLSYWGMESVPNHYGQIRMESFVVGEDTTSFILGDCHEMLRTETIDILLAAVAQSFRRVFTDRRMPTIYNEGHGRESWDSNLDLSRTVGWFTTLCPLQVDECSGSDFVDTTKRVKDLRRKIKDNGRSYFARSLLQANNTEPSDFPVPLEIVFNYLGRLQQLERDDSLFKHYGEAFDEEKFRLAGDMGSDTPRFALLEISALVVNDKLQVSFTYNRQMQRESQIFQWISECRRVLEIDVLRFKDTVPEPTLSDFPLLPITYDGLKKLTSTTLPRAGVKTFSQVEDIYPCSSVQEGILLSQLRDPSAYMFHVIFEVRSPGGSGKVDPNMLRSAWSAVINRHPILRTLFIDSNYANGTFDQLVLRKVDEGAIILHCNDSDALVKLDTIKLSEINAGRCPKLLHQLTVCSTDSGRVLIKLEMNHAIIDGGSVDLLLRDLAMAYKHQLPEGSGPHFSDYIKFVRGKSQSQALSHWRQYLSDAHPCHLTFSEGTGGSRQLGSVMVPFSRYSELQQFCEKNSVTLANLTLAAWAIVLQSFTGSNDVCFGYPSAGRDAPVPGIQDAVGIFLNMLCCRVRFSPGKTLLEVSKTVQDDYIKNLPYQDCSLASIQHELGQKELFNTTISIQNHHAVSEESGNDLLSFDVQTAHDPTEYPVTVNVETAKGHEGILLRYWTDAVPEDKANNLANAIAHIFSCFIDKPSQLVSELDLRGGQLMKGGQFIDSKSLQELIDRRVTEIISQMLKEGTLAIPAADNGKGQFKGTTRAQPAKVTIKARGMHRERDLSDSTATLTEDHSKLMEPEKRLWKIWCSALGLASDTIQRQASFFKLGGDSITAMKMVSAAREDGLTLTMADVFNNPVFEDMLAAISASNSSSALEPDSPADSNNEKPAEPPRLVELERNPPPPEISLLKTVPLNDSALQDGICPKIGVFKGGIADVLPVTDFQAMSITATLFKSRWMLNYFFFDGRGSLDLRRLRESLLRVVDAFDILRTVFVCFNGQFFQVVLRKIRPNIFVHETEKNLDEYTEYLQQQDREQEARQGEQYVKFYIVKKKGSNHHRILIRMSHAQFDGLCLPTIMSAIKLGYEGSTLPPAPSFANYMRMLSGAITPDHYQHWTNLLKGSKMTQVIRRTEENTYRYIGAFREQRKTLEIQPSVLENVTIATVMQAAWAVTLAKLSAQSDVVFGLTISGRNTSIPGIENTVGPCLNVIPIRVTFKEGWTGVDLFRYLQDQQIANMPFEALGFREIIRRCTDWPSSTYFTTSVFHQNVEYEGQMQLDNTTYRMGGAGVVDNFTDMTLFSKSSSDGKLGVSLGYSDKGPIGPKFAAKVLSMVCDTVQSLLANSRVGLPSPSMLRSLPCQSVHDVPGMTDEMFLSTHLKDRSISDILVHSDVVTQAWQQVLPRNNADEPQSSFQLDSSFFELGGDVFDMAQVVWLLEQEGLQVHIEDLLEHPTFLGQMAVLTLHNSRTSDSMEEIVPVEEIPLPAARTGTSSSLGKALTLAKKVTRWSALSARG

(19) United States
(12) Patent Application Publication
(10) Pub. No.: US 2026/0242430 A1
(43) Pub. Date: Aug. 20, 2026

(54) NOVEL FUNGAL DERIVED COMPOUNDS

(71) Applicant: Aalborg Universitet, Aalborg Øst (DK)

(72) Inventors: Teis Esben Søndergaard, Randers NV (DK); Peter Kristensen, Tranbjerg (DK); Klaus Ringsborg Westphal, Sæby (DK); Trine Aalborg, Gistrup (DK); Balázs Delényi, Aalborg (DK); Thea Laulund Lunden, Aalborg (DK)

(21) Appl. No.: 19/161,431

(22) PCT Filed: Feb. 29, 2024
(86) PCT No.: PCT/EP2024/055185

§ 371 Date: Sep. 2, 2025

(30) Foreign Application Priority Data
Mar. 3, 2023 (DK) PA202370118

(51) Int. Cl. C07K 14/37; A61K 39/00; C07K 16/14; C12N 9/22; C12N 15/11; C12N 15/90; G01N 33/569

(52) U.S. Cl. CPC C07K 14/37; C07K 16/14; C12N 9/226; C12N 15/11; C12N 15/902; G01N 33/56961; A61K 39/00; C07K 2317/565; C07K 2317/569; C07K 2317/92; C12N 2310/20; G01N 2333/37; G01N 2469/10

(57) ABSTRACT

The present invention relates to compounds of Non-Ribosomal Peptide Synthetase and methods of analysing them. In particular the present invention relates to the use of these compounds in vaccines and antigen-binding molecules targeting these compounds.

### TECHNICAL FIELD OF THE INVENTION

The present invention relates to compounds of Non-Ribosomal Peptide Synthetase and methods of analysing them. In particular, the present invention relates to the use of these compounds in vaccines and antigen-binding molecules targeting these compounds.

### BACKGROUND OF THE INVENTION

Fungi produce several secondary metabolites (SM) to endure changing surroundings, and one of the major tasks is to adapt to alterations in humidity and to maintain the turgor pressure. Fungi have developed an arsenal of safeguards build into the fungal cell wall and plasma membranes to prevent premature sporulation under unsuitable growth conditions, to break of surface tension raising the hyphae from aquatic environment and to protect the fungi from flooding. A growing collection of orthologous, though highly diverged, Non-Ribosomal Peptide Synthetase (NRPS) genes have been coupled to fungal surface hydrophobicity in five filamentous ascomycetes, and could be a new addition to the group of universally occurring SM.

An improved method of identifying these compounds would be advantageous, and in particular a more efficient and/or reliable method of identifying the NRPS would be advantageous.

### SUMMARY OF THE INVENTION

The inventors of the present invention have surprisingly realized that these secondary metabolites, referred to herein as compounds, are present in almost all fungi, that they are present at the extracellular surface of these fungi, and that these compounds are accessible for specific antigen recognition, such as by antibodies.

As these compounds are present at the cellular surface, and since their structures are not readily derivable, i.e. due to their non-ribosomal production site, representative gene sequences cannot be found—the present inventors have devised an ingenious novel way of targeting fungi.

This novel targeting of fungi can thus be used to generate vaccines and interventions to combat infections as well as novel ways of detecting the fungi. We thus present herein methods of identifying and analysing these compounds, the compounds themselves, their use as vaccines and antigen-targeting molecules with specificity against such compounds.

Thus, an object of the present invention relates to a method of identifying compounds produced from NRPS in fungi. In particular, it is an object of the present invention to provide novel compounds derived from fungi.

Thus, one aspect of the invention relates to a method of analysing the compound produced by a fungus-derived Non-Ribosomal Peptide Synthetase (NRPS), wherein the method comprises:
a. Providing a first fungus, such as Apiospora, and a second fungus such as the same species as the first fungus, such as the same strain as the first fungus, such as Apiospora;
b. genetically modifying the expression of the NRPS in the first fungus, such as knocking out or overexpressing the NRPS gene;
c. growing the first fungus and the second fungus under conditions suitable for the production of compounds, such as compounds of NRPS;
d. recovering extracts from the first fungus and the second fungus;
e. analysing the extracts; such as by LC-MS and/or NMR spectroscopy; and
f. comparing the analyses of the extracts between the first fungus and the second fungus, thereby identifying compounds of the NRPS.

Another aspect of the present invention relates to a compound identified by the method as described herein.

Another aspect of the present invention relates to a compound produced from the Non-Ribosomal Peptide Synthetase (NRPS).

Another aspect of the present invention relates to a compound, such as a secondary metabolite produced from a Non-Ribosomal Peptide Synthetase (NRPS) of a fungus, such as the Non-Ribosomal Peptide Synthetase 4 (NRPS4) of Apiospora.

Another aspect of the present invention relates to the compound as described herein for use as a vaccine.

Another aspect of the present invention relates to the use of the compound as described herein as a surfactant.

Another aspect of the present invention relates to an antigen-binding molecule having affinity against the compound as described herein, such as an antibody, or an antigen-binding fragment thereof.

Another aspect of the present invention relates to an antigen-binding molecule having affinity against the extracellular surface of Apiospora, such as an antibody, or an antigen-binding fragment thereof.

Another aspect of the present invention relates to use of the antigen-binding molecule as described herein in a method of detecting a fungus, such as Apiospora, such as an in vitro method.

Another aspect of the present invention relates to the use of the antigen-binding molecule as described herein in the treatment of a non-human infection, such as an infection on trees, such as a tree selected from the group consisting of eucalyptus and bamboo.

Another aspect of the present invention relates to a genetically modified fungus, such as Apiospora, such as an Apiospora overexpressing the NRPS4, such as wherein the NRPS4 gene has been knocked out.

Another aspect of the present invention relates to a host cell, such as a fungus, comprising the NRPS4 gene of Apiospora, with the proviso that the NRPS4 gene of Apiospora has been artificially introduced into the fungus.

Another aspect of the present invention relates to a method of producing the compound as described herein, wherein the method comprises:
a. Providing an NRPS gene from a fungus, such as the NRPS4 gene of Apiospora;
b. inserting a promoter upstream of the NRPS gene and/or upstream of the NRPS cluster specific transcription factor, such as a constitutively active promoter, thereby providing an activated NRPS gene;
c. Introducing the activated NRPS gene into a suitable host cell, such as a fungus, such as Apiospora;
d. Growing the host cell under conditions that
   a. allows for the production of the enzyme encoded by the NRPS4 gene; and
   b. allows for the production of the compound;
e. recovering the compound, such as by LC, HPLC.

Another aspect of the disclosure is thus a method of selecting antigen-binding molecules with specificity towards an NRPS derived compound, the method comprising:
a. Providing a library for selecting antigen-binding molecules, such as a library comprising phages;
b. Providing a fungus expressing a compound as described herein, and providing a purified version of the compound;
c. Contacting the library with the fungus;
d. Contacting the fungus with the purified version of the compound;
e. Retrieving antigen-binding molecules with specificity against the compound, such as retrieving phages with specificity against the compound.

### BRIEF DESCRIPTION OF THE FIGURES

FIG. 1 shows antiSMASH prediction of modular structure and A domain substrates of the Ap. Arundinis (AAU773) NRPS4. PCP domains, C domains, A domains, and E domains are indicated.

FIG. 2 shows sectional view of the disrupted NRPS4 gene (cyan) in the Ap. arundinis (AAU773) KO::NRPS4 strain genome assembly with gene annotation.

FIG. 3 shows inactivation of NRPS4 in Ap. arundinis (AAU773) abolishes surface water repellence. A) Comparison of 7- and 30-day old cultures of WT and KO::NRPS4 cultures and stereo (top right) and light (bottom right) microscope images of 30-day old mycelium with conidia (bar=10 μm). B) Time-study of surface hydrophobicity with methylene blue dyed water droplets. Asterisks indicate wrapping of the plate in parafilm.

FIG. 4 shows droptest on Aspergillus fumigatus. 50 μl of water add as a droplet on either WT (left) and Pes1 Knock Out mutant (Right). The phenotype is similar to the phenotypes in Fusarium graminearum and Apiospora arundinis.

FIG. 5 shows Commassie stained SDS gel from expression and purification of the antibodies AP1, AP2, AP3, AP4, AP7 and AP8 in lane 1 of all gel the MW marker was loaded. A) Ni-NTA purification of AP1 in lane 2-5 fractions 3-6 from elution, AP2 in lane 6-9 fractions 3-6 from elution, B) Ni-NTA purification of AP3 in lane 2-5 fractions 3-6 from the elution, AP4 in lane 6-9 fractions 3-6 from the elution, and C) Ni-NTA purification of AP7 in lane 2-7 fractions 5-10 from the elution, AP8 in lane 8-11 fractions 5-8 from the elution.

FIG. 6 shows octet measurements on the AP1 antibody. The curve fitting was performed under the assumption that the concentration of Apiosporin was 100 nM. A), and B) respectively shows the association and dissociation performed on KO fungi, C) and D) respectively shows the association and dissociation performed on WT fungi.

FIG. 7 shows octet measurements on the AP2 antibody. The curve fitting was performed under the assumption that the concentration of Apiosporin was 100 nM. A), and B) respectively shows the association and dissociation performed on KO fungi, C) and D) respectively shows the association and dissociation performed on WT fungi.

FIG. 8 shows octet measurements on the AP3 antibody. The curve fitting was performed under the assumption that the concentration of Apiosporin was 100 nM. A), and B) respectively shows the association and dissociation performed on KO fungi, C) and D) respectively shows the association and dissociation performed on WT fungi.

FIG. 9 shows octet measurements on the AP4 antibody. The curve fitting was performed under the assumption that the concentration of Apiosporin was 100 nM. A), and B) respectively shows the association and dissociation performed on KO fungi, C) and D) respectively shows the association and dissociation performed on WT fungi.

FIG. 10 shows identification of Apiosporin after immunoprecipitation with the selected sdAbs AP1, AP2 and AP7. The mass spec analysis identify Apiosporin by their typical masses indicated in the figure.

FIG. 11 shows immunofluorescent staining using AP1 as the primary antibody and and ALFA tag antibody as the secondary (Alexa 647), in addition the calcofluor white stain was used to visualize the cell wall of KO fungi. Pictures were taken at a 100× magnification. A) cell wall, B) Apiosporin specific sdAb, C) merge. No clearly staining in septa. Scalebar 20 μm.

FIG. 12 shows immunofluorescent staining using AP1 as the primary antibody and and ALFA tag antibody as the secondary (Alexa 647), in addition the calcofluor white stain was used to visualize the cell wall of WT fungi. Pictures were taken at a 100× magnification A) cell wall, B) Apiosporin specific sdAb, C) merge. Staining is clearly shown in septa between two hyphae. Scalebar 50 μm.

The present invention will now be described in more detail in the following.

### DETAILED DESCRIPTION OF THE INVENTION

### Definitions

Prior to discussing the present invention in further details, the following terms and conventions will first be defined:

### Fungus

The term a “fungus” or “fungi” or “funguses” is to be understood as any member of the group of eukaryotic organisms that includes microorganisms such as yeasts and molds, as well as mushrooms. These organisms are classified as a kingdom, separately from the other eukaryotic kingdoms, which by one traditional classification include Plantae, Animalia, Protozoa, and Chromista. A characteristic that places fungi in a different kingdom from plants, bacteria, and some protists is chitin in their cell walls.

### Amino Acids

“amino acids” are understood in the present context as comprising both proteinogenic amino acids and non-proteinogenic amino acids. In particular, amino acids of the present invention can common proteinogenic amino acids,
The common forms of amino acids have a zwitterionic structure,
with —NH₃⁺ (—NH₂⁺— in the case of proline) and —CO₂⁻ functional groups attached to the same C atom, and are thus α-amino acids. With the exception of achiral glycine, natural amino acids have the L configuration, and are the only ones found in proteins during translation in the ribosome.

The L and D convention for amino acid configuration refers not to the optical activity of the amino acid itself but rather to the optical activity of the isomer of glyceraldehyde from which that amino acid can, in theory, be synthesized (D-glyceraldehyde is dextrorotatory; L-glyceraldehyde is levorotatory).

In particular embodiments, the compound of the disclosure comprises an alternating D/L alpha architecture in the backbone.

### Degenerate Sequence

The term “degenerate sequence” is understood as a nucleotide sequence encoding the same sequence of amino acids as the sequence of which it is a degenerate of, i.e. by the use of different codons. Thus, two degenerate sequences will encode the same protein.

### Subject

The term “mammal” comprises humans of all ages, other primates (e.g., cynomolgus monkeys, rhesus monkeys); mammals in general, including commercially relevant mammals, such as cattle, pigs, horses, sheep, goats, mink, ferrets, hamsters, cats and dogs, as well as birds. Preferred mammals are humans.

### Sequence Identity

The term “sequence identity” is here defined as the sequence identity between genes or proteins at the nucleotide, base or amino acid level, respectively. Specifically, a DNA and an RNA sequence are considered identical if the transcript of the DNA sequence can be transcribed to the corresponding RNA sequence.

Thus, in the present context, “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide at the corresponding position in the second sequence, then the molecules are identical in that position. The percent identity between the two sequences is a function of the number of identical positions shared by the sequences (i.e., % identity=#of identical positions/total #of positions (e.g., overlapping positions)×100). In one embodiment, the two sequences are the same length.

In another embodiment, the two sequences are of different length and gaps are seen as different positions. One may manually align the sequences and count the number of identical amino acids. Alternatively, alignment of two sequences for the determination of percent identity may be accomplished using a mathematical algorithm. Such an algorithm is incorporated into the BLASTN and BLASTX programs of (Altschul et al. 1990). BLAST nucleotide searches may be performed with the NBLAST program, to obtain nucleotide sequences homologous to a nucleic acid molecule of the invention. BLAST protein searches may be performed with the BLASTX program, to obtain amino acid sequences homologous to a protein molecule of the invention.

To obtain gapped alignments for comparison purposes, Gapped BLAST may be utilized. Alternatively, PSI-Blast may be used to perform an iterated search that detects distant relationships between molecules. When utilizing the BLASTN, BLASTX, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted. An embodiment of the present invention thus relates to sequences of the present invention that has some degree of sequence variation.

It should be noted that embodiments and features described in the context of one of the aspects of the present invention also apply to the other aspects of the invention.

All patent and non-patent references cited in the present application, are hereby incorporated by reference in their entirety.

### Nonribosomal Peptide-Synthetase

The compounds of the present invention, such as nonribosomal peptides, are synthesized by a specialized nonribosomal peptide-synthetase (NRPS) enzyme. The NRPS genes for a certain enzyme are arranged in gene clusters in fungus. The enzymes are organized in modules that are responsible for the introduction of one additional amino acid. Each module consists of several domains with defined functions, separated by short spacer regions of about 15 amino acids. There are also NRPS enzymes that serve as a scaffold for other modifications to the substrate to incorporate unusual amino acids.

The modules of the NRPS of the present disclosure will comprise the domains A, PCP, and C. To provide the specific stereochemistry of the compounds as presented herein, every other module will comprise an E-domain.
A: Adenylation,
PCP: Thiolation and peptide carrier protein with attached 4′-phospho-pantetheine,
C: Condensation forming the amide bond, and
E: Epimerization into D-amino acids,

The modules are responsible for the production of the compound, and the modules as well as the functioning of the NRPS will typically follow the structure of a starting stage where an amino acid is loaded, elongation stages, where additional amino acids are loaded and attached to the previous amino acid via the condensation domains, and optionally configured as D-amino acids, and lastly a termination stage where the production of the compound is terminated. NRPS often follows the collinearity rule, that the number of modules reflects the length of the peptide in linear NRPSs, but iterative modules can be found within the NRPS family.

The NRPS of the invention will comprise between 3-10 modules, such as any of 3, 4, 5, 6, 7, 8, 9, or 10 modules. Preferably, the modules will be identified as modules with alternating C-A-PCP-E and C-A-PCP organizations.

Most importantly, the NRPS of the present invention comprises a domain architecture wherein an epimerization (E) domain is present in alternating modules.

As such, all identified orthologues as presented in example 6 have these alternating E-domains, and the identification of this pattern in the model is a part of the method to identify homologous NRPS.

As presented further down below, the NRPS may be from any fungus.

As presented by examples 1-5 Apiospora produces a compound as presented herein. NRPS in Apiospora may be referred to as NRPS4. Thus, in a specific embodiment, the NRPS may be from Apiospora.

The inventors have identified the specific NRPS in Apiospora arundinis AAU773. In one embodiment of the present disclosure, the NRPS4 is encoded by the gene according to SEQ ID NO: 1, or a degenerate sequence thereof. In one embodiment of the present disclosure, the NRPS4 is encoded by the cDNA according to SEQ ID NO: 2, or a degenerate sequence thereof.

Since some sequence variance will be expected when investigating other strains of Apiospora, it is of relevance that some sequence variety may exist in these genes and proteins.

In one embodiment of the present disclosure, the NRPS4 is the protein according to SEQ ID NO: 4, or a variant thereof having at least 80% sequence identity. Such as at least 85% identity, such as 90% identity, such as 95% identity, such as 99% identity, such as even 99.9% identity to the NRPS4 according to SEQ ID NO: 4.

In another embodiment, the sequence identity is assessed upon the individual domains. As such, any domain of an NRPS may show least 80% sequence identity, such as at least 85% identity, such as 90% identity, such as 95% identity, such as even 99% identity to the corresponding domain as present in the NRPS4 according to SEQ ID NO: 4.

However, when comparing between different fungi, the variance will be much larger and vary depending on the relatedness of the fungi in comparison. For species not closely associated phylogenetic sequence identity levels between NRPS homologues of those of the present invention will likely be too low to infer homology. For instance, an analysis between the NRPS in Apiospora and Aspergillus yielded a sequence identity score of 35%, which could be matched in a comparison between the Apiospora NRPS4 and any other NRPS in Aspergillus with an E domain. I.e. protein identity comparisons cannot for all homologues conclusively predict homology. Typically though it is expected that the highest amount of identity can be found between A-domains.

As presented by example 7, Pes1 was identified in Aspergillus Fumigatus to an NRPS according to the invention, and thus produces a compound as presented herein. Thus in another embodiments of the inventio, the NRPS is encoded by the cDNA according to SEQ ID NO: 40, or a degenerate sequence thereof.

In one embodiment of the present disclosure, the NRPS is the protein according to SEQ ID NO: 41, or a variant thereof having at least 80% sequence identity. Such as at least 85% identity, such as 90% identity, such as 95% identity, such as 99% identity, such as even 99.9% identity to the NRPS according to SEQ ID NO: 41. In another embodiment, the sequence identity is assessed upon the individual domains. As such, any domain of an NRPS may show least 80% sequence identity, such as at least 85% identity, such as 90% identity, such as 95% identity, such as even 99% identity to the corresponding domain as present in the NRPS according to SEQ ID NO: 41.

In one embodiment of the present disclosure, the NRPS is the protein according to TERG_01444, or a variant thereof having at least 80% sequence identity. Such as at least 85% identity, such as 90% identity, such as 95% identity, such as 99% identity, such as even 99.9% identity to the NRPS according to TERG_01444. In another embodiment, the sequence identity is assessed upon the individual domains. As such, any domain of an NRPS may show least 80% sequence identity, such as at least 85% identity, such as 90% identity, such as 95% identity, such as even 99% identity to the corresponding domain as present in the NRPS according to TERG_01444. TERG_01444 is hereby incorporated by reference.

### Methods of Analysing and Producing

As presented, the disclosure provides methods of analysing the compounds derived from a specific NRPS.

One aspect of the invention relates to a method of analysing the compound produced by a fungus-derived Non-Ribosomal Peptide Synthetase (NRPS), wherein the method comprises:
a. Providing a first fungus, such as Apiospora, and a second fungus such as the same species as the first fungus, such as the same strain as the first fungus, such as Apiospora;
b. genetically modifying the expression of the NRPS in the first fungus, such as knocking out or overexpressing the NRPS gene;
c. growing the first fungus and the second fungus under conditions suitable for the production of compounds, such as compounds of NRPS;
d. recovering extracts from the first fungus and the second fungus;
e. analysing the extracts; such as by LC-MS and/or NMR spectroscopy; and
f. comparing the analyses of the extracts between the first fungus and the second fungus, thereby identifying compounds of the NRPS.

The skilled person will understand that the method finds use for any fungi, such as any of the fungi disclosed herein.

The method finds use in different ways, and many methods are known to modify the expression of proteins, thus in one embodiment of the present disclosure, the knocking out is achieved by gene editing, such as site-directed mutagenesis, such as the use of CRISPR/Cas9. In another embodiment of the present disclosure, overexpression is achieved by inserting a promoter upstream of the NRPS gene and/or upstream of the NRPS cluster specific transcription factor, such as a constitutively active promoter.

Since it is vital that the NRPS comprises an E-domain in every other module, the method may also include a step of identifying an E-domain in every other module of the NRPS.

Such a method may also include to submit the genome to antiSMASH for biosynthetic gene cluster prediction and/or to AUGUSTUS for gene prediction. Additionally, it may thus be advantageous to compare the domain architecture of the predicted NRPS genes, and finally identify the NRPS displaying alternating epimerization domains.

Thus the method may essentially also be referred to as a method of analysing the compound produced by a fungus-derived Non-Ribosomal Peptide Synthetase (NRPS), wherein the NRPS comprises an E-domain in every other module.

### Compound

The method introduced above can thus be used in the analysis and identification of compounds produced from a specific NRPS.

The present disclosure thus also provides this compound, hence another aspect of the present invention relates to a compound identified by the method as described herein. Preferably, the compound is an isolated compound, meaning that the compound is substantially free of any impurities or contaminants.

A further aspect of the present invention relates to a compound produced from the Non-Ribosomal Peptide Synthetase (NRPS). Preferably, the compound is an isolated compound.

Still another aspect of the present invention relates to a compound, such as a secondary metabolite, produced from a Non-Ribosomal Peptide Synthetase (NRPS) of a fungus, such as the Non-Ribosomal Peptide Synthetase 4 (NRPS4) of Apiospora.

Since the compounds, when produced in its natural form in the fungus is referred to as secondary metabolites, the compound of the present disclosure can also be referred to as secondary metabolites. In one embodiment of the present disclosure, the compound is a secondary metabolite. In one embodiment of the present disclosure, the compound is a non-ribosomal peptide.

The compound may also be provided as a salt. In one embodiment of the present disclosure, the compound is a salt thereof, such as a pharmaceutically acceptable salt thereof. In another embodiment, the compound is lyophilized.

As presented above, the compounds of the present disclosure are not produced by the ribosome, however are primarily composed of amino acids. In one embodiment of the present disclosure, the compound comprises amino acids, such as proteinogenic amino acids and/or non-proteinogenic amino acids. In one embodiment of the present disclosure, the compound comprises a mixture of proteinogenic amino acids and non-proteinogenic amino.

Since the compounds resemble peptides, their backbone will mostly consist of peptide bonds. In one embodiment of the present disclosure, at least two of the amino acids, such as all amino acids, are coupled by peptide bonds formed between the α-nitrogen atom of one amino acid and the carbonyl carbon of a second amino acid. In one embodiment of the present disclosure, the compound is a peptide.

However as can be seen from the compounds of Formula I-VI, the backbone may also comprise at least one amino acids bonded in a different way. Thus in another embodiment, at least one amino acids is bonded via a non-peptide bond to the compound.

The compound of the present disclosure is likely produced as a linear peptide, which may be made cyclic additionally. In one embodiment of the present disclosure, the compound is cyclic, i.e. forming a ringstructure. In one embodiment of the present disclosure, the compound is linear. Since the ring-structure may be opened again by the breakage of the bonds in the backbone, a number of linearized compounds can be produced therefrom.

A vital aspect of the compounds of the present disclosure is the alternating structure present in the backbone of the compound. In one embodiment of the present disclosure, the amino acids in the compound are positioned in a stereochemistry according to the general formula (L-D)ⁿ and/or (D-L)ⁿ, wherein n is an integer from 3-10, such as any of 3, 4, 5, 6, 7, 8, 9, or 10, and D and L represent amino acids in either a D or L conformation. Thus, when a compound is comprised of 6 amino acids the compound will either follow the structure L-D-L-D-L-D, or D-L-D-L-D-L. It is thus given, that when a compound comprises an uneven number, such as seven, of amino acids, and is present in a cyclic form, at least one pair of amino acids will be joined together in either a D-D or a L-L configuration, however when counting from the first to the last amino acids, such as the seventh, the structure L-D-L-D-L-D-L, or D-L-D-L-D-L-D will be apparent. In one embodiment of the present disclosure, each consecutive amino acid has sidechains in an opposing enantiomer in relation to the previous amino acid. In one embodiment of the present disclosure, the compound comprises an D/L alpha architecture in the backbone.

As presented by example 4, the absolute stereochemical configuration was solved by Marfey's method resulting in a D/L-alpha-architecture alternating in a backbone-cyclic peptide.

Since NRPS' can comprises a varying number of modules, the resulting compounds thereof can comprise a varying number of amino acids. In one embodiment of the present disclosure, the compound comprises 3-10 amino acids, such as any of 3 amino acids, such as 4 amino acids, such as 5 amino acids, such as 6 amino acids, such as 7 amino acids, such as 8 amino acids, such as 9 amino acids, or such as 10 amino acids, preferably such as 7 amino acids.

Since additional bonds or chemical groups in the compound may be produced or inserted by other enzymes, it it also possible that the compound is processed further, after having been assembled by the NRPS. In one embodiment of the present disclosure, the compound have been further modified by at least one additional enzyme.

As presented by examples 1-4 a number of specific compounds have been identified in Apiospora, thus in one embodiment of the present disclosure, the compound is a compound represented by Formula I-VI selected from:

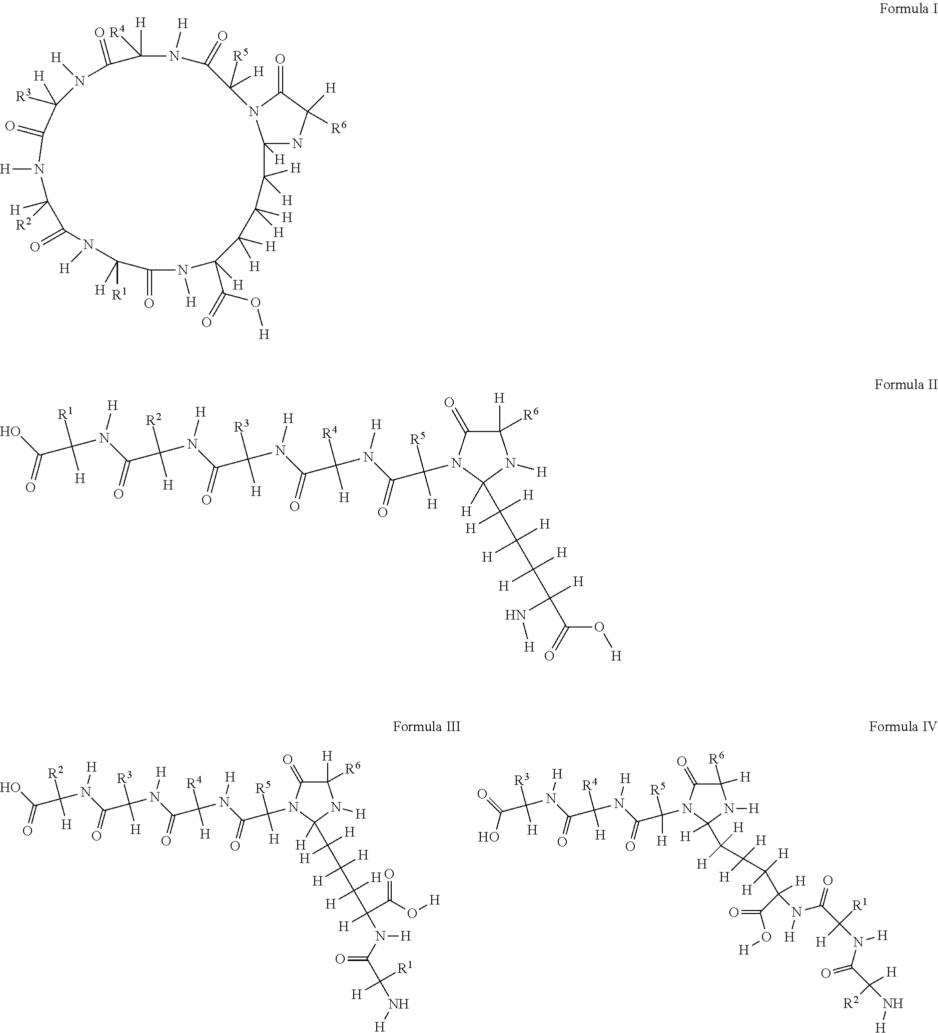

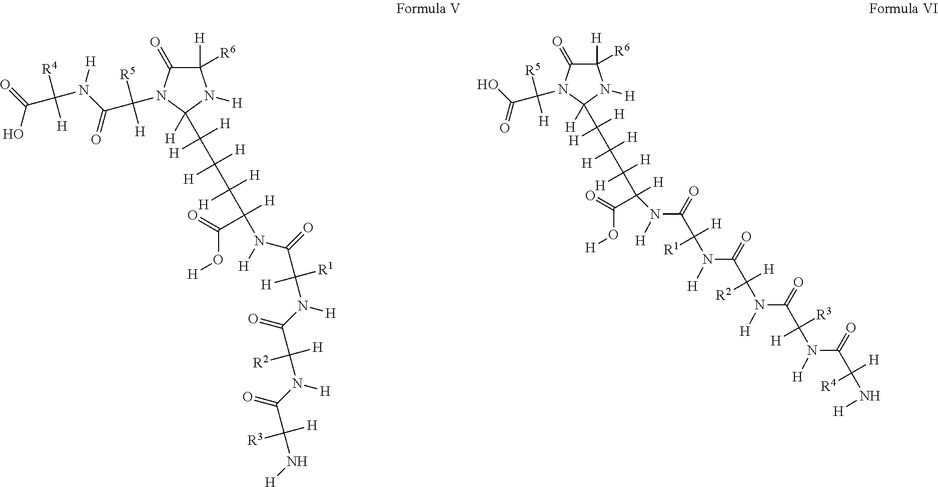

wherein R¹ is L-Tyrosine or D-Tyrosine; R² is L-Leucine or D-Leucine; R³ is L-Alanine, D-Alanine, L-Leucine or D-Leucine; R⁴ is L-Leucine or D-Leucine; R⁵ is L-Leucine or D-Leucine; and R⁶ is L-Leucine or D-Leucine, and the Lysine is a L-Lysine or a D-Lysine, with the proviso that when R¹, R³, R⁵ are L-enantiomers, R², R⁴, and R⁶ are D-enantiomers, or when R¹, R³, R⁵ are D-enantiomers, R², R⁴, and R⁶ are L-enantiomers.

Even more specific embodiments is thus a compound represented by Formula VII or VIII selected from:

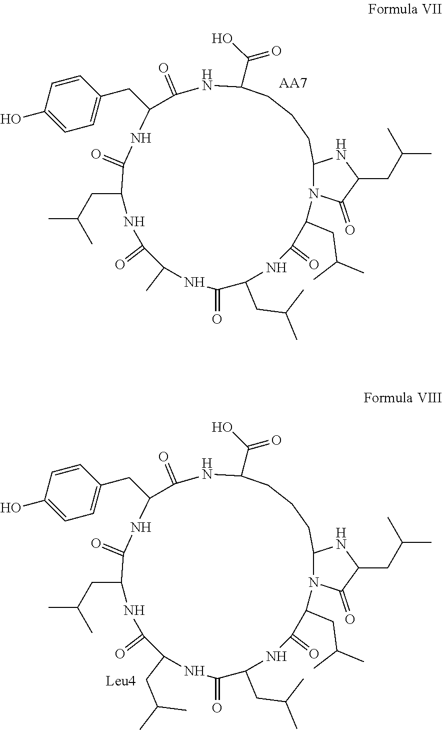

with the proviso that when R¹, R³, R⁵ are L-enantiomers, R², R⁴, and R⁶ are D-enantiomers, or when R¹, R³, R⁵ are D-enantiomers, R², R⁴, and R⁶ are L-enantiomers.

### Fungi of the Disclosure

The NRPS of the present disclosure are found in almost all fungi, and as introduced above are responsible for controlling the response to humid environments. Thus, the methods presented herein finds use in all fungi. A simple test to identify the capability of a fungus to response to humid environments is to use the water droplet retention time study presented in example 3. In one embodiment of the present disclosure, the fungus is able to repel water, such as in a water droplet retention time study. In another embodiment, the fungus is not able to repel water when the NRPS gene has been knocked out.

As described in the example 6, all ascomycetes fungi comprise the NRPS. Thus, In one embodiment of the present disclosure, the fungus is any ascomycetes fungi such as
Any Fusarium species, such as F. graminearum and F. avenaceum;
any Aspergillus species such as A. fumegatus and A. flavus;
any trichophylon species such as T. rubrum;
any Penicillium species such as P. chrysogenom and P. rubens;
Any Cochliobolus species such as C. heterotrophus, all Chaetomium species such as C. globosum.

The currently only known fungus which does not express a compound as disclosed herein is Neurospora crassa, thus in one embodiment of the present disclosure, the fungus is not Neurospora crassa. Thus, in a specific embodiment NRPS are found in all fungi, with the proviso that Neurospora crassa does not comprise a relevant NRPS.

It is very relevant to identify compounds of fungi that causes disease in mammalians, thus all mammalian disease-relevant fungus are of interest. In one embodiment of the present disclosure, the fungus is a mammalian disease-relevant fungus, such as a human disease-relevant fungus, such as a fungus selected from Aspergillus, Trichophyton, Candida, and Fusarium.

Specifically to name some of the infections caused by these fungi; Aspergillus may cause infections to the lungs, Trichophyton may cause infections on the skin, hair and nails, Candida may cause candidiasis, thrush, vaginitis, and Fusarium is involved with causing irritations due to their presence on contact lenses.

As presented by examples 1-5, Apiospora produces a compound as presented herein. In one embodiment of the present disclosure, the fungus is Apiospora. In another embodiment of the present disclosure, the Apiospora is Apiospora arundinis. In a specific embodiment of the present disclosure, the Apiospora arundinis is Apiospora arundinis AAU773. NRPS in Apiospora may be referred to as NRPS4.

The inventors have identified the specific NRPS in Apiospora arundinis AAU773. In one embodiment of the present disclosure, the NRPS4 is encoded by the gene according to SEQ ID NO: 1, or a degenerate sequence thereof. In one embodiment of the present disclosure, the NRPS4 is encoded by the cDNA according to SEQ ID NO: 2, or a degenerate sequence thereof.

Since some sequence variance will be expected when investigating other strains of Apiospora, it is of relevance that some sequence variety may exist in these genes and proteins.

In one embodiment of the present disclosure, the NRPS4 is the protein according to SEQ ID NO: 4, or a variant thereof having at least 80% sequence identity. More details are presented above.

As presented by example 7, Aspergillus produces a compound as presented herein. In one embodiment of the present disclosure, the fungus is Aspergillus. In another embodiment of the present disclosure, the Aspergillus is Aspergillus Fumigatus. NRPS in Aspergillus may be referred to as Pes1. In one embodiment of the present disclosure, the NRPS is encoded by the cDNA according to SEQ ID NO: 41, or a degenerate sequence thereof.

As presented by example 8, Trichophyton produces a compound as presented herein. In one embodiment of the present disclosure, the fungus is Trichophyton. In another embodiment of the present disclosure, the Trichophyton is Trichophyton rubrum. NRPS in Trichophyton may be referred to as TERG_01444.

### Uses and Vaccines

As presented above, a much striking finding by the present inventors is that the compounds as presented above, are responsible for providing a resistance against humid environmenst (example 3), and are thus present at the surface. As seen in example 5, these compounds are accesible to antibodies.

Thus, antibodies against these compounds can be produced. Furthermore, recombinant antibodies can be produced, for use in therapy and detection. Additionally, this show that these compounds can be bound by antibodies both when inserted in a cell membrane, however also when present in solution. These compounds are also present on the extracellular surface of fungus showing that the epitopes are accesible to the immune system, and that vaccines can be generated by using NRPS-derived compounds as presented herein.

Thus, another aspect of the present invention relates to the compound as described herein for use as a vaccine.

It is thus evident, that one of such uses is for combating viral infections. In one embodiment of the present disclosure, the vaccine is a vaccine against a fungus infection. In another embodiment of the present disclosure, the fungus infection is an infection with the fungus of which the compound is originally derived, such as where the NRPS is present in the genome of the fungus of which the compound is originally derived.

To facilitate a reaction towards the immune system, the compounds may be conjugated to compounds readily recognized by the immune system. In one embodiment of the present disclosure, the compound is coupled to an adjuvant, such as a toxin.

Due to the compound's ability to present both hydrophilic and hydrophobic properties, another important aspect of the invention is to salvage these properties and use the compounds as surfactants.

Another aspect of the present invention relates to the use of the compound as described herein as a surfactant. In one embodiment, the surfactant is an emulsifier.

### Antigen-Binding Molecules Targeting the Compound

Also provided herein are the antigen-binding molecules capable of binding to the compounds as presented herein. Preferably, the antigen-binding molecules are isolated antigen-binding molecules.

Another aspect of the present invention relates to an antigen-binding molecule having affinity against the compound as described herein, such as an antibody, or an antigen-binding fragment thereof.

Another aspect of the present invention relates to an antigen-binding molecule having affinity against the extracellular surface of a fungus, such as Apiospora, such as an antibody, or an antigen-binding fragment thereof.

In one embodiment of the present disclosure, the extracellular surface of a fungus, such as Apiospora, is a compound as described herein, present on the surface of the fungus. In another embodiment of the present disclosure, the antigen-binding molecule can bind to a solubilized form of the compound, such as when the compound is not being bound to a cellular membrane, such as the membrane of a fungus.

In particular the antigen-binding molecule may also be an antigen-binding portion thereof.

Many different types of antigen-binding molecules exist, and thus it is not intended to be limited to the specific arrangement providing the specificity. In one embodiment of the present disclosure, the antigen-binding molecule is a molecule selected from the group consisting of an antibody, a scFv, a single domain antibody (sdAb) or nanobody, a VHH, an isolated single variable domain, an affibody, a DARPin, a monobody, an anticalin, an affilin, an affimer type 1 molecule, an affimer type 2 molecule, an affitin, an alphabody, an anticalin, an avimer, a fynomer, a kunitz domain peptide, a nanoclamp, and an aptamer.

The antigen-binding molecule may also be coupled with other binding specificities and thus in another aspect the disclosure relates to a multi-specific antibody, such as a bispecific antibody, comprising at least one of the antigen-binding molecules or antigen-binding portions thereof as described herein.

In a preferred embodiment, the antigen-binding molecule is an antibody or a fragment thereof, such as a scFv, capable of binding the compound as presented herein.

In one embodiment of the present disclosure, the antigen-binding molecule comprises:
a heavy chain variable region comprising a CDR 1 region, a CDR 2 region, and a CDR 3 region; and
a light chain variable region comprising a CDR 1 region, a CDR 2 region, and a CDR 3 region.

In one embodiment of the present disclosure, said antigen-binding molecule is selected from the group consisting of:
a) an antigen-binding molecule comprising:
   a CDR 1 region according to SEQ ID NO: 7, a CDR 2 region according to SEQ ID NO: 8 and a CDR 3 region according to SEQ ID NO: 9;
b) an antigen-binding molecule comprising:
   a CDR 1 region according to SEQ ID NO: 12, a CDR 2 region according to SEQ ID NO: 13 and a CDR 3 region according to SEQ ID NO: 14;
c) an antigen-binding molecule comprising:
   a CDR 1 region according to SEQ ID NO: 17, a CDR 2 region according to SEQ ID NO: 18 and a CDR 3 region according to SEQ ID NO: 19
d) an antigen-binding molecule comprising:
   a CDR 1 region according to SEQ ID NO: 2, a CDR 2 region according to SEQ ID NO: 23 and a CDR 3 region according to SEQ ID NO: 24;
e) an antigen-binding molecule comprising:
   a CDR 1 region according to SEQ ID NO: 27, a CDR 2 region according to SEQ ID NO: 28 and a CDR 3 region according to SEQ ID NO: 29;
f) an antigen-binding molecule comprising:
   a CDR 1 region according to SEQ ID NO: 32, a CDR 2 region according to SEQ ID NO: 33 and a CDR 3 region according to SEQ ID NO: 34; or
g) an antigen-binding molecule comprising:
   a CDR 1 region according to SEQ ID NO: 37, a CDR 2 region according to SEQ ID NO: 38 and a CDR 3 region according to SEQ ID NO: 39.

In a preferred embodiment of the present disclosure, said antigen-binding molecules are single domain antibodies.

In a specific embodiment of the present disclosure, said antigen-binding molecule is selected from the group consisting of:
a) the single domain antibody according to SEQ ID NO: 6;
b) the single domain antibody according to SEQ ID NO: 11;
c) the single domain antibody according to SEQ ID NO: 16;
d) the single domain antibody according to SEQ ID NO: 21;
e) the single domain antibody according to SEQ ID NO: 26;
f) the single domain antibody according to SEQ ID NO: 31; or
g) the single domain antibody according to SEQ ID NO: 36.

In a specific embodiment of the present disclosure, said antigen-binding molecule is encoded by nucleic acids selected from the group consisting of:
a) the nucleic acid according to SEQ ID NO: 5;
b) the nucleic acid according to SEQ ID NO: 10;
c) the nucleic acid according to SEQ ID NO: 15;
d) the nucleic acid according to SEQ ID NO: 20;
e) the nucleic acid according to SEQ ID NO: 25;
f) the nucleic acid according to SEQ ID NO: 30; or
g) the nucleic acid according to SEQ ID NO: 35.

We thus also provide the nucleic acids encoding these antigen-binding molecules, and methods of producing the antigen-binding molecules. Thus in a specific embodiment, the present disclosure relates to vectors, such as plasmids, comprising any of the nucleic acids selected from the group consisting of SEQ ID NO: 5, SEQ ID NO: 10, SEQ ID NO: 15 SEQ ID NO: 20, SEQ ID NO: 25, SEQ ID NO: 30, SEQ ID NO: 35, host cells comprising the nucleic acids or vectors.

Another aspect of the present invention relates to use of the antigen-binding molecule as described herein in a method of detecting a fungus, such as Apiospora, such as an in vitro method.

Another aspect of the present invention relates to the antigen-binding molecule for use as a medicament.

Another aspect of the present invention relates to the antigen-binding molecule for use in the treatment of an infection with a mammalian disease-relevant fungus, such as a human disease-relevant fungus, such as a fungus selected from Aspergillus, Trichophyton, Candida, and Fusarium.

The antigen-binding molecules may also be coupled to other compounds and thus specifically be able to deliver compounds. Such compounds can both be used for mammalian treatment, as well as treating infections plants and trees. In one embodiment of the present disclosure, the antigen-binding molecule is an antibody-drug conjugate, such as coupled to a toxin or a medicament. In one embodiment of the present disclosure, the antigen-binding molecule is coupled to a toxin.

Another aspect of the present invention relates to the use of the antigen-binding molecule as described herein in the treatment of a non-human infection, such as an infection on trees, such as a tree selected from the group consisting of eucalyptus and bamboo. In a particular embodiment of the present disclosure, the non-human infection is thus an infection with an Apiospora, having infected a plant or a tree.

The antigen-binding molecules as described above, may advantageously be selected as presented in example 5, by the use of a library. i.e. by first selecting against the fungus, and secondly in a competitive selection procedure where the bound phages of the fungus are washed off by addition of the compound as disclosed herein. Another aspect of the disclosure is thus a method of selecting antigen-binding molecules with specificity towards an NRPS derived compound, the method comprising:
a. Providing a library for selecting antigen-binding molecules, such as a library comprising phages;
b. Providing a fungus expressing a compound as described herein, and providing a purified version of the compound;
c. Contacting the library with the fungus;
d. Contacting the fungus with the purified version of the compound;
e. Retrieving antigen-binding molecules with specificity against the compound, such as retrieving phages with specificity against the compound.

### Modified Microorganisms and Methods of Producing the Compound

The NRPS responsible of providing the compounds according the present disclosure may be artificially moved between different fungi, such as to express an NRPS from one species, in another species, or even entirely move it to another genus. Thus, another aspect of the present invention relates to a genetically modified fungus, such as Apiospora, such as an Apiospora overexpressing the NRPS4, such as wherein the NRPS4 gene has been knocked out. Another aspect of the present invention relates to a genetically modified fungus, such as Aspergillus, such as an Aspergillus overexpressing the NRPS, such as wherein the NRPS gene has been knocked out. Another aspect of the present invention relates to a genetically modified fungus, such as Trichophyton, such as an Trichophyton overexpressing the NRPS, such as wherein the NRPS gene has been knocked out.

A KO fungus may preferably display a lowered ability to respond to humid environments, thus in one embodiment of the present disclosure, the mycelium of the genetically modified fungus, such as Apiospora, such as Aspergillus, such as Trichophyton, does not repel water, such as when studied in a water droplet retention time study.

Another aspect of the present invention relates to a host cell, such as a fungus, comprising the NRPS4 gene of Apiospora, with the proviso that the NRPS4 gene of Apiospora has been artificially introduced into the fungus. Another aspect of the present invention relates to a host cell, such as a fungus, comprising the Pes1 gene of Aspergillus, with the proviso that the Pes1 gene of Aspergillus has been artificially introduced into the fungus. Another aspect of the present invention relates to a host cell, such as a fungus, comprising the TERG_01444 gene of Trichophyton, with the proviso that the TERG_01444 gene of Trichophyton has been artificially introduced into the fungus.

Another aspect of the present invention relates to a host cell, such as a fungus, comprising the NRPS gene of a fungus, with the proviso that the NRPS gene of the fungus has been artificially introduced into the fungus.

Another aspect of the present invention relates to a host fungus, comprising the NRPS gene of a different fungus, with the proviso that the NRPS gene of the different fungus has been artificially introduced into the host fungus.

It may be advantageous to express the compounds as described herein in large amounts, thus in another aspect of the present invention relates to a method of producing the compound as described herein, wherein the method comprises:
a. Providing an NRPS gene from a fungus, such as the NRPS4 gene of Apiospora;
b. inserting a promoter upstream of the NRPS gene and/or upstream of the NRPS cluster specific transcription factor, such as a constitutively active promoter, thereby providing an activated NRPS gene;
c. Introducing the activated NRPS gene into a suitable host cell, such as a fungus, such as Apiospora;
d. Growing the host cell under conditions that
   a. allows for the production of the enzyme encoded by the NRPS4 gene; and
   b. allows for the production of the compound;
e. recovering the compound, such as by LC, HPLC.

The skilled person will understand that the method finds use for any fungi, such as any of the fungi disclosed herein.

The skilled person will be able to design the method, so as to obtain a compound having properties in, such as purity, that the skilled person so desires. I.e. when the compound is for injection into a mammal, such as human, a high purity is sought after, whereas when a compound if for use as a surfactant, it may be provided in a less pure quality.

The invention will now be described in further details in the following non-limiting examples.

### EXAMPLES

Fungi: Apiospora arundinis (Isolate AAU773) full genome sequenced by Nanopore technology.

### Example 1

Identification of NRPS4 in Apiospora arundinis

### Aim:

To identify the NRPS 4 gene in a new strain of Apiospora arundinis (AAU773)

### Methods:

A whole-genome assembly of Ap. arundinis AAU773 was submitted to antiSMASH for biosynthetic gene cluster prediction and AUGUSTUS for gene prediction. The domain architecture of all predicted NRPS genes were compared to known NRPS4 homologues, where only one displayed similar alternating epimerization domains found to be conserved in homologues. BLASTp was performed to establish homology, but did not produce conclusive evidence of phylogeny.

### Results:

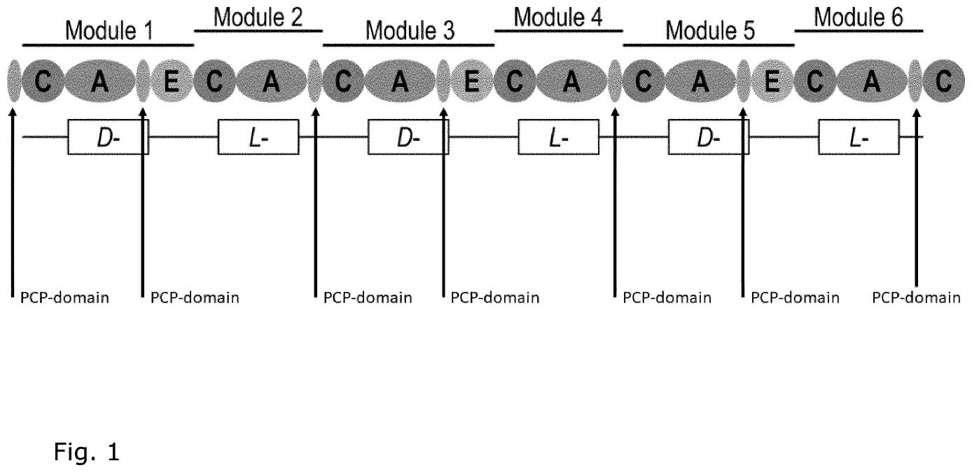

The modular structure of NRPS4 in Ap. Arundinis (AAU773) includes an initial PCP domain followed by six modules with alternating C-A-PCP-E and C-A-PCP organizations and a single C-terminal C domain. The antiSMASH substrate specificity analysis predicted alternating L- to D-isomerizations by E domain-holding modules (See FIG. 1).

### Conclusion:

NRPS4 is encoded in 25,182 nt gene_12562 of Apiospora arundinis (AAU773) (position 1483306; 1584550 in in-house assembly).

### Example 2

Generation of Apiospora arundinis (AAU773) NRPS4 Knock-Out Strain by CRISPR/Cas9 Genome Editing

### Aim:

To inactivate the NRPS4 biosynthetic gene in Apiospora arundinis by generating a knock-out strain.

### Methods:

For gene knock-out in Apiospora we used a plasmid-free cloning system using commercially available Cas9 and RNA components from Integrated DNA Technologies to preassemble RNP complexes in vitro and introduce these directly into the Apiospora protoplast by PEG-mediated transformation, thereby not relying on exogenous promoter and terminator sequences for Cas9 endonuclease activity, but only for expression of antibiotic resistance conferring enzyme activity.

Enzymatic Protoplastation of Ap. Arundinis (AAU773)

Ap. arundinis (AAU773) mycelium was harvested from a five-day old YPG liquid culture by filtering through double-layered autoclaved nappy liner and rinsing with a small amount of KC buffer (60 g/L KCl, 2.2 g/L citric acid monohydrate, pH 6.2). The mycelium was washed in 40 mL KC buffer in a 50 mL Falcon tube by 30 sec vigorous shaking and centrifugation at 4000 rcf, 4° C. for 8 min. Agglomerates of mycelium were homogenized by adding four 3 mm glass beads and glass sands to the Falcon tube and bead beating by vortexing for 2 min. The suspension was settled to pellet glass beads and large remaining agglomerates, and the mycelium suspended in the top layer was decanted to a new 50 mL Falcon tube. The volume in the bead beating tube was adjusted to 20 mL with KC buffer, and the homogenization repeated twice. The mycelium homogenate was pelleted by centrifugation at 4000 g, 4° C. for 8 min to separate cells from lysate, and the supernatant was discarded. The mycelium was resuspended in 7.5 mL of 0.2 μm sterile filtered 25 mg/mL Lysing Enzyme from Trichoderma harzianum (Glucanex) (Sigma Aldrich, Cas no. L142-10G), 65 mg/mL VinoTaste PRO® maturation 6-10 g/hl (novozymes) in KC buffer and the reaction was incubated horizontally in a 50 mL Greiner tube at 30° C., 150 rpm for 3 hrs. Protoplastation progress was monitored by light microscopy. Following confirmation of adequate protoplast concentration, the suspension was chilled on ice for 5 min to stop the reaction. Protoplasts were separated from mycelial debris into a fresh 50 mL Falcon tube by filtering through double-layered autoclaved nappy liner and rinsing with a small amount of cold KC buffer. The total suspension volume was adjusted to 20 mL with KC buffer, and protoplasts were pelleted by centrifugation at 600 g, 4° C. for 5 min. The supernatant was discarded, and the protoplasts resuspended gently in 10 mL KC buffer and 10 mL cold STC buffer (5.5 g/L CaCl2·2H2O, 219 g/L D-sorbitol, 10 mL/L Tris-HCl (pH 7.5)). Protoplasts were pelleted by centrifugation at 600 g, 4° C. for 5 min and resuspended in 20 mL STC buffer. Protoplast concentration was determined by hemocytometer (Bürker, 0.100 mm, Marienfeld) and protoplasts pelleted by centrifugation at 600 g, 4° C. for 5 min. Protoplasts were resuspended in STC buffer to final concentration of 2·107/mL. One volume of STC cryopreservation buffer (20% (w/v) glycerol in STC buffer) was added for every one volume of protoplasts. 500 mL aliqouts were frozen to −80° C.

### Generation of Homology-Directed Repair Template DNA by Overlap-Extension PCR

The NRPS4 knock-out mutant was prepared by RNP-mediated CRISPR/Cas9 in vivo gene editing using a selection marker-based homology directed repair (HDR) approach. To inactivate the gene, a crRNA targeting a 5′ end proximal exon of the gene was designed and a linear donor DNA (dDNA) repair template with 1.1 kb homology regions flanking the programmed cut-site was prepared. The dDNA included a hygromycin phosphatase expression cassette placed in-between the homology bands, facilitating genomic integration of the resistance marker gene at the Cas9 cut-site and disruption of the NRPS4. The dDNA repair template was constructed from three DNA fragments by a single overlap extension PCR reaction using 36 bp overlaps between each of the annealing fragments to function as primers for the overlap extension elongation. The three fragments were 1 kb homology regions flanking the target integration site, which were amplified from genomic DNA of Ap. arundinis (AAU773), and the hygromycin resistance cassette, amplified from purified plasmid pRF-HU2E. The 36 bp overlap regions were introduced to the homology band amplicon through primer overhangs. Overlap extension PCR was performed in two reactions, 1) primer-free fragment fusion by overlap extension, and 2) amplification of the fusion gene with standard PCR. The fusion gene dDNA product was purified from agarose gel and sequenced by nanopore sequencing.

### RNP Assembly and Protoplast Transformation

For RNP assembly, Cas9 Nuclease V3 was diluted in 1× Cas9 activity buffer (20 mM HEPES, 150 mM KCl, 8.2 mM MgSO4, 0.1 mM EDTA, 0.5 mM DTT, PH 7.5, 0.2 μm sterile filtered) to final concentration 1 μg/μL. RNP complexes were prepared as 1.5 μL of 33 μM gRNA duplex, 0.75 μL of 1 μg/μL Cas9, 11 μL 1× Cas9 activity buffer (final volumes 13.25 μL). RNP suspension was incubated at ambient temperature for 5 min to allow complex formation.

The RNP complex suspension was co-transformed with 1 μg dDNA into 200 μL 1*10⁷/mL Apiospora arundinis protoplasts by PEG-mediated transformation, by adding 25 μL 60% PEG 4000 buffer (60% (w/v) PEG 4000, 50 mM CaCl2, 10 mM Tris-HCl, pH 7.5) was added to all transformations, which were then incubated on ice for 50 min. Following incubation, another 1250 μL 60% PEG 4000 buffer was added to each transformation. Tubes were then incubated at ambient temperature for 20 min to allow uptake of RNP and DNA into protoplasts. The total volume of each transformation reaction was adjusted to 3 mL with 2×STC buffer. Each total transformant suspension was then plated on osmotic recovery plates (YPG with 1 M D-sorbitol, and supplemented with 300 μg/mL hygromycin B for selection marker-based HDR clonings), forming a homogeneous liquid surface layer. The plates were dried for 30 min and incubated in the dark at 25° C. to allow regeneration of cell wall and formation of transformant colonies. After 3 days, positive transformants were purified by restreaking onto YPG with 300 μg/mL hygromycin B.

### Whole-Genome Sequencing of Positive Transformants

Transformants were verified by Nanopore whole-genome sequencing. High-molecular weight genomic DNA was purified from liquid cultures of Apiospora arundinis (AAU773) KO::NRPS4 and was then nanopore sequenced using a MinION flow cell. A DNA library was prepared from ≈1500 ng Ap. Arundinis (AAU773)

KO::NRPS4 genomic DNA, following the Native barcoding DNA (with EXP-NBD104, EXPNDB114, and SQK-LSK109) protocol (ONT) for barcoding and library preparation. The MinION flow cell, displaying 900 active pores, was loaded and sequencing was performed on the MinION sequencer for 42 h, obtaining 927.95 K reads, covering 7.74 Gb. Guppy was used to basecall the raw data, sort according to barcode, trim adaptor sequences, and generate fastq data files. Filtlong was used to filter <10 kb reads and discard 10% lowest quality read bases. The filtered reads were mapped onto a modified whole-genome sequence of Ap. arundinis (AAU773), holding the HygR gene insert at the targeted integration site, using Minimap2. CLC Genomics Workbench 20 was used to visualize the mapped genome. A genome assembly was generated from the Guppy fastq files. Filtlong was used to trim reads to ≥10 kb and ≥80% mean quality score threshold. An initial assembly was formed using Minimap2, to map reads onto each other, and Racon (v1.3.3) and Medaka (v1.0.1) to polish the assembly.

### Results:

The KO::NRPS4 genome was assembled to 15 contigs spanning 45.94 Mb in total. The N50 of 5.30 Mb and N99 of 0.75 Mb indicate good contiguity of the assembly, also apparent in the number of contigs. Annotation showed that the insert had been incorporated correctly at the target locus and that no ectopic integration events had occurred. Subsequent cultivation of the mutant on non-selection medium also confirmed that the generated mutant was genetically stable.

### Conclusions:

We successfully prepared an NRPS4 knock-out strain of Apiospora arundinis (AAU773) by targeted integration of a hygromycin resistance cassette in an upstream exonic region of the gene.

### Example 3

### Phenotype of Apiospora Knockout Strain

### Aim:

To determine the phenotype of the Ap. arundinis (AAU773) KO::NRPS4 strain.

### Methods:

Triplicate YPG plates of Ap. arundinis (AAU773) KO::NRPS4 and WT were inoculated with mycelium and incubated in the dark in a ventilated drawer at room temperature, to avoid entrapment of condensation. To assay the surface hydrophobicity, a 30 μL droplet of methylene blue in nucelase-free water was added onto the mycelial surface on days 7, 11, 14, 18, 21, 25, 30, and 35 over a 35-day period. Droplets were incubated on the mycelium for 15-45 min prior to photographing the plates. An additional set of triplicate plates of KO::NRPS4 and WT Ap. arundinis were incubated top-up in the dark in a standard incubator at 25° C. for 30 days, allowing lid condensation. Plates were photographed intermittently, and mycelium was analyzed in light microscope and stereo microscope after 30 days.

### Results:

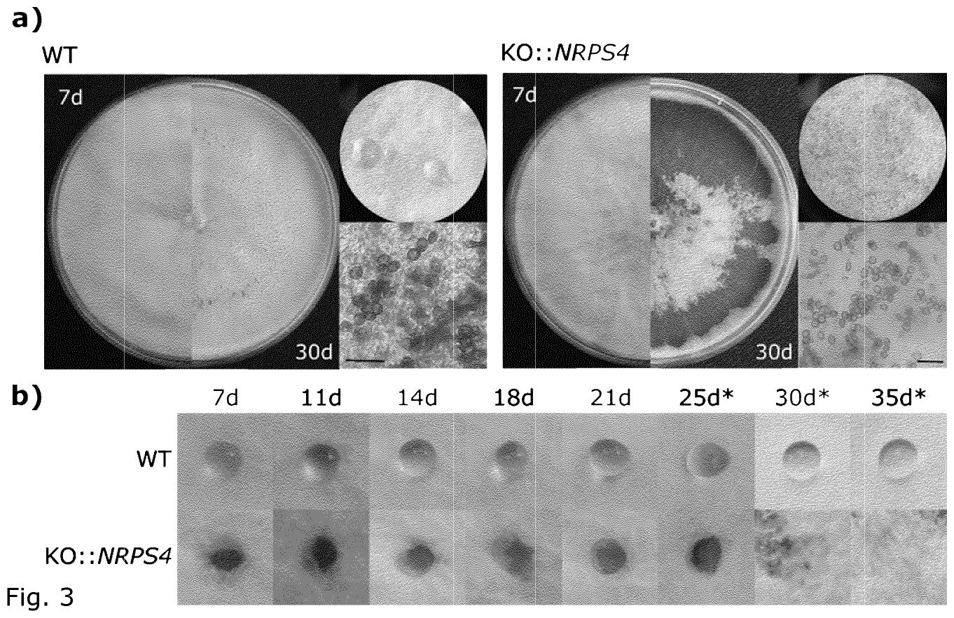

After seven days, the mycelial structure of the mutant appeared fluffy and was brittle to handle compared to the denser WT mycelium (FIG. 3a). Prolonged incubation produced a collapsed mutant mycelium, the collapse spanning the entire surface after 30 days. The mycelium was wet to handle, and inspection under stereo microscope showed that the surface properties of the KO::NRPS4 mutant were aberrated as the mycelium had soaked in water from the humidity and condensation droplets. The diminished water repellence of the mutant mycelium, indicates that the hydrophobic surface properties of the mycelium, and possibly the spores, was lost a result of NRPS4 inactivation. The WT remained unaffected by the surface retained water droplets, concordant with a notable reduction of surface hydrophobicity in the mutant. Under light microscope, there were no apparent morphological differences between the WT and KO::NRPS4 conidia, both strains successfully producing dark brown, globose conidia along conidiophore mother cells. However, whereas the cell wall structures of individual hyphae could be discerned in the WT strain with nested clusters of conidia in dry, airy mycelium, the mycelium of the mutant appeared rather as a dense mass holding dispersed clusters of conidia.

The alteration of surface hydrophobicity imposed by NRPS4 knock-out was further examined in a water droplet retention time study (FIG. 3b), where the cultures' ability to resist the surface tension of methylene blue dyed water droplets on their surfaces was tested over a 35-day period. Over the entire assay duration, the droplets were able to penetrate the KO::NRPS4 mycelium, and absorption could be almost immediate. Contrary to this, the WT completely repelled the droplets, which were retained on the mycelial surface until dried out (approximately 10 days in a ventilated drawer). Inactivation of NRPS4 hence completely abolishes surface hydrophobicity of the mycelium in young cultures and the ability to repel water is not gained by means of compensation as the culture ages.

### Conclusion:

The surface of the Ap. arundinis (AAU773) mycelium is unable to repel water in the KO::NRPS4, whereas water droplets can be completely retained on the WT surface.

### Example 4

### Derivation of Apiosporin

Aim: To Identify the Composition and Structure of the Compounds Produced from NRPS4

### Methods:

Cultivation of A. arundinis (AAU773)

A total number of 204 plates of LB agar medium were inoculated with A. arundinis (AAU773) and incubated at 25° C. in the dark for 14 days.

### Extraction of Compounds

After cultivation the plates were dismantled into pieces of approximately 5×5 mm and transferred to ten 5 L bluecap flasks, covered with ethylacetate:dichlormethane:methanol (3:2:1) with 1% formic acid, extracted ultrasonically for 45 min and filtered through Miracloth (Merck). After filtration two distinct phases were observed and the phases were separated in a separation funnel. The 600 mL brown light phase was phase separated once again after addition of 200 mL ethyl acetate and 100 mL dichlormethane before rotary evaporation to almost dryness. A volume of 10 μL was transferred to a HPLC-vial for analysis. The remaining liquid was removed by lyophilisation. The extracts were analysed by HPLC-HRMS on a C6-phenyl column with an initial concentration of solvent B of 10% increasing to 99% over ten minutes and kept at 99% for five minutes. The approximately 3.2 L yellow heavy phase was rotary evaporated to a volume of approximately 7 mL.

### Purification of Selected Compounds

The compounds of interest from the yellow heavy phase were isolated with Waters AutoPurifier HPLC with QDa mass spectrometer. Using a preparative C8 column (19×250 mm, 5 μm, XBrigde OBD, Waters) with a flow of 20 mL/min with an initial gradient of solvent B of 30% increasing to 80% from the 1.5 minute mark to the 10 minute mark, increasing to 99% over four minutes and kept at 99% for one minute. A total of eleven different masses were selected for isolation by single ion monitoring of [M+H]+ _0:5 m=z. Before lyophilisation, pH was adjusted to pH 6-8. The lyophilized samples were reconstituted in methanol until completely dissolved. The samples were analysed by Waters AutoPurifier HPLC/MS on a C6-phenyl column (4.6×250 mm, 5 μm, Gemini, Phenomenex) with an initial concentration of solvent B of 20% increasing to 99% over 15 minutes and kept for five minutes. Two fractions containing compounds of (referred to as apiosporin A) 814.5 [M+H]+ and one of two fractions containing compounds of (referred to as apiosporin B) 856.5 [M+H]+ were purified on a Waters AutoPurifier HPLC/MS using a preparative C8 column (19×250 mm, 5 μm, XBrigde OBD, Waters) with a flow of 20 mL/min and an initial concentration of solvent B of 35% increasing to 60% from the 1.5 minute mark to the 13 minute mark and increasing to 99% over two minutes. The purified fractions were lyophilised.

### Structure Elucidation

The lyophilised purified peptides were dissolved in 600 μL DMSO-d6 and analyzed with BRUKER AVIII-600 MHz NMR spectrometer with a cryogenically cooled triple resonance 5 mm probe with z-gradient. Spectra were calibrated using solvent signals of 1H (2.50 ppm) and 13C (39.5 ppm). Spectra recorded for structure elucidation were 1H-NMR, 13C-NMR, TOCSY (90 ms mixing time), COSY, ROESY(250 ms mixing time), [1H, 13C]- and [1H, 15N]-HSQC, and [1H, 13C]- and [1H, 15N]-HMBC. Experiments were run at a temperature of 308.1 K. Concentrations of the compounds were determined with PULCON. Data was processed and analyzed in TopSpin v.3.6.1 (Bruker) and Computer Aided Resonance Assignment (CARA) v.1.8.4.2.

### Marfeys Reaction.

From each sample of apiosporin A, 0.1 mL was diluted 1:10 with water (HiPerSolv CHROMANORM, VWR), flash frozen in liquid nitrogen and lyophilized. The dried samples were reconstituted in 1 mL 6 M HCl (Sigma-Aldrich) and hydrolyzed at 110° C. for 24 h in a N₂ atmosphere. The hydrolyzed samples were dried under a flow of N₂ and reconstituted in 0.1 mL water (HiPerSolv CHROMANORM, VWR). The samples, as well as 5 μmol of each amino acid standard (I-Leu, d-Leu, I-Ala, d-Ala, I-Tyr, d-Tyr, I-Lys, d-Lys) were derivatized with FDAA (Sigma-Aldrich). The derivatized amino acids were analyzed by HPLC.

### Results:

By comparing the extracts of cultures of WT and KO::NRPS4 cultures, we were able to identify the specific compounds that are produced from NRPS4 in Apiospora. A number of compounds were produced however in particular fractions containing compounds of (apiosporin A) 814.5 [M+H]+ and fractions containing compounds of (apiosporin B) 856.5 [M+H]+ were investigated in more detail, since they were found to be abundant.

Comparison of the 1H-NMR spectra of the four apiosporin A compounds, revealed similar overall features, yet with differences in the chemical shifts.

The TOCSY spectra suggested the presence of seven spin systems, but only five of these were revealed in the N^{H}-section of the spectrum. For the remaining two, no N^{H} shifts were observed, and the spin systems were revealed in the H^{α} section. Combined with COSY, ROESY, [¹H,¹³C]-HSQC and -HMBC as well as [¹H,¹⁵N]-HSQC and -HMBC, several of these spin systems were identified as the proteinogenic amino acid leucine, one was identified as alanine and one was identified as tyrosine while the last system was identified as a lysine resembling non-proteinogenic amino acid.

In order to determine the order of the amino acids in the peptide chain, a sequential walk based on ROE correlations of N^{H} and H^{α} was conducted. The ROE correlations between N^{H} and H^{α} protons in all six compounds revealed the backbone amino acid sequence Leu2-Leu3-(Ala/Leu) 4-Leu5-Tyr6-Lys7. The residual connection between Leu1 and Leu2 was discovered through HMBC correlations between C^{α} of Leu1 and H^{α} of Leu2. The HMBC correlations confirmed the sequence from the ROE correlations as well as suggesting a correlation between Lys7 He and Leu1 C through Leu1 N. This link between Lys7 He and Leu1 N was also suggested by the presence of weak [¹H,¹³C]-HMBC signals between Lys7 H^{ε} and Leu1 C^{α}, and Leu1 H^{α} and Lys7 C^{ε}.

Another key observation regarding elucidation of the final structures of apiosporin were the observations of a strong [¹H,¹³C]-HMBC signal between Leu2 H^{α} and Lys7 C^{ε}. The presence of this signal suggested a bond between Leu2 N and Lys7 C^{ε}. The only way to satisfy both observations as well as comply with the molecular formula is through a 4-imidazolidinone ring between the backbone of Leu1 and Leu2, and the sidechain of Lys7. This completes the cyclic structure of apiosporin.

Thus, in summary, the structures of the most abundant compounds were identified and the NRPS4 of Apiospora mainly produce apiosporin A and apiosporin B:

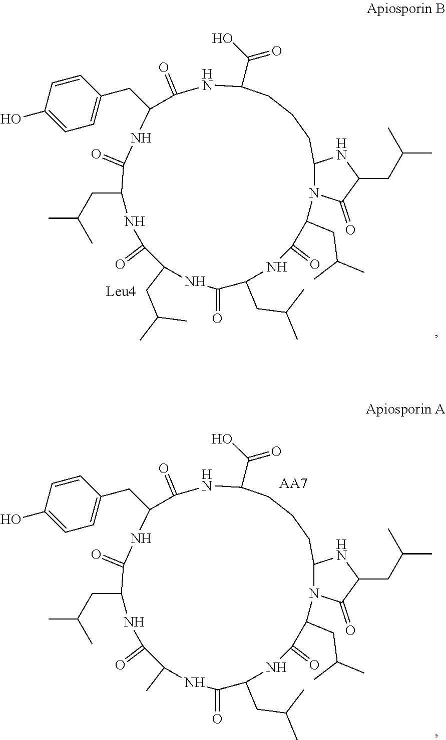

with an alternating D/L alpha architecture in the backbone. The compounds can also be linearized, and can thus be represented by the formulas I-VIII as described herein.

### Conclusion:

The structures of the most abundant products of NRPS4 in Apiospora were derived, and was able to be represented by general formulas describing both compounds.

### Example 5

Selection of Single Domain Antibodies (sdAbs) on Apiospora arundinis (Isolate AAU773)

Aim: the aim is to generate antibody fragments that specifically recognise Apiosporin presented on the surface of Apiospora arundinis (Isolate AAU773). By using an in vitro recombinant antibody generation platform building on phage display (Mandrup OA, Friis NA, Lykkemark S, Just J, Kristensen P (2013) A Novel Heavy Domain Antibody Library with Functionally Optimized Complementarity Determining Regions. PLOS ONE 8 (10)), it will be possible to
1) generate antibody fragments forming the basis for future development of immunotherapies against fungal diseases; and
2) validate that Apiosporin is presented on the surface of Apiospora arundinis (Isolate AAU773) and identify which part of the peptide will be optimal for vaccine development.

Material used for selection: The selection of sdAbs is based on a single framework library with a diversity of around 10e8 different sequences. The library is described in the reference cited above, and contain randomisation in the CDR2 and CDR3 of the variable heavy chain constituting the sdAbs. The variability is constructed using trinucleotide synthesis to mimick the diversity observed in naturally occurring human immunoglobulins.

Apiospora arundinis (Isolate AAU773) were grown on an agar plug of 4 mm in diameter.

### Description of the Selection Procecure:

One day prior to selection, an overnight culture of the TG-1 bacteria were established by picking a colony from TG-1 growing on minimal plate and inoculating it into 50 ml 2×TY media. The TG-1 culture was incubated to reach an OD600 of 0.4, which is the point were the bacteria would be in the exponential growth phase and expressing the F-pilus. The TG-1 bacteria strain is a suppressor E. coli strain and it would read the amber stop codon (TAG) between the sdAb gene and the geneIII in the phagemid, thereby producing phage particles with sdAb antibody fused to the N-terminal of pIII.

Selection was carried out on Apiospora arundinis (Isolate AAU773) presented on a plug of agar.

Two Apiospora arundinis (Isolate AAU773) was mixed with the human Predator sdAb antibody library in two separate selection experiments (100 ul with a concentration of 10e12 phage/ml) and incubated on a rotation wheel for 1 hr. Unbound materials were removed by pipetting of the supernatant. 1000 ul PBS were added and incubated for 5 min, supernantant removed as before. The washing was repeated 7 times.

One of the two selections (A) were used for specific elution by adding apiosporin (0.1 mg/ml adding 1 ml) while the other (B) were treated in the same manner but without adding apiosporin.

Following 1 hour of incubation of (A) and (B) on a rotation wheel, the plugs were collected and the supernatant containing apiosporin specific eluted phage particles (A) or non-specifically eluted phage (B) were retrieved.

On half of the elution volume from each selection were saved, while the other half were incubated on an agar plug containing Apiospora arundinis (Isolate AAU773) KO fungi allowing depletion of phage particles presenting sdAbs binding non-specifically to Apiospora arundinis (Isolate AAU773). After 1 hour incubation the plugs were retrieved and the supernatants collected.

To remove phage particles not presenting an sdAb on their surface, each supernatant were incubated by adding 50 μl trypsin-PBS (10 mg/ml trypsin stock solution) and incubation for 10 min. 100 μl of the eluted phages were added to 200 μl TG1 culture with an OD600 of 0.4 (the exponential growth phase) and allowed to infect for 30 min at 37° C. The infected bacteria were plated on TYE plates containing 100 μg/ml ampicillin and 1% glucose and grown overnight at 37° C.

The bacterial colonies were counted and retrieved by scaping in 2 ml 2×TY containing Amp.

From the homogenous population of bacterial colonies a 50 ml culture of 2×TY/Amp/Glucose were inoculated to reach an OD600 of 0.5.

The KM13 helperphage were added to a final concentration of 10e9 phage/ml. After incubation for 30 min at 37 C the bacteria were retrieved by centrifugation at 4000 g for 10 min. The bacterial pellet was resuspended in 50 ml 2×TY/Amp/Kan, and phage rescue were performed by incubation overnight at 30 C shaking at 190 RPM.

After overnight incubation the supernatant were retrieved by centrifugation at 4000 g for 30 minutes. The phage particles were precipitated from the supernatant by addition of ⅕ volume of a solution containing 20% PEG600 and 2.5 M NaCl. The precipitated phage was collected by centrifugation at 4000 g for 30 min. Phage particles were resuspended in 1 ml PBS constituting the polyclonal output from the first selection.

A second and third round of selection were performed by following the same protocol as above.

### Results:

| | |
|---|---|
| | Number of |
| Elution | colonies formed |

First round of selection:

| | | |
|---|---|---|
| Apiospora arundinis | (B) | 4920 |
| (Isolate AAU773) knock out | | |
| Apiospora arundinis | (B) | 6450 |
| (Isolate AAU773) wildtype | | |
| Apiospora arundinis | (A) | 12860 |
| (Isolate AAU773) knock out | | |
| Apiospora arundinis | (A) | 12640 |
| (Isolate AAU773) wildtype | | |

Second round of selection

| | | |
|---|---|---|
| Apiospora arundinis | (B) | 157600 |
| (Isolate AAU773) knock out | | |
| Apiospora arundinis | (B) | 75800 |
| (Isolate AAU773) wildtype | | |
| Apiospora arundinis | (A) | 259600 |
| (Isolate AAU773) knock out | | |
| Apiospora arundinis | (A) | 294000 |
| (Isolate AAU773) wildtype | | |

(A) indicates specific elution by adding Apiosporin (0.1 mg/ml adding 1 ml)
(B) indicates elution in PBS.

When eluting specifically with Apiosporin, compared to just eluting in PBS, we see an increased number of antibodies being eluted in the first and second round.

In the second round we see an enrichment of binders for all elutions, this indicate specific binding to cell wall presented apiosporin. The increased number of antibodies being eluted with PBS alone could indicate that an enrichment of antibodies binding to other targets on the fungi is also seen.

### Conclusion:

The results show two major findings:
Firstly, antibodies against Apiosporin can be produced. This highlights that recombinant antibodies can be produced, and be used in therapy and detection. Additionally, this show that the NRPS-derived compounds can be bound both when bound in a cell membrane, however also when present in solution.
Secondly, Apiosporin is present on the extracellular surface of Apiospora arundinis showing that the epitopes will be accesible to the immune system, and that vaccines can be generated by using NRPS-derived compounds

### Example 6

Aim: To identify the presence of the NRPS identified in example 1 across other fungi

Methods: To further identify the presence of NRPS having alternating E-domains in other fungi, we conducted the method as described for example 1, on a large collection of different fungi and thus investigated a large number of species from different fungi selected from Fusarium, Aspergillus, trichophylon, Penicillium, and Cochliobolus.

### Results:

All Fusarium species were found to have the gene encoding the NRPS, such as F. graminearum and F. avenaceum.

All Aspergillus species were found to have the gene encoding the NRPS, such as A. fumegatus and A. flavus.

All trichophylon species were found to have the gene encoding the NRPS, such as T. rubrum.

All Penicillium species were found to have the gene encoding the NRPS, such as P. chrysogenom and P. rubens.

All Cochliobolus species were found to have the gene encoding the NRPS, such as C. heterotrophus, all Chaetomium species such as C. globosum.

Thus, all fungi, in particular all ascomycetes fungi, comprises the NRPS having alternating E-domains.

### Conclusion:

Thus, we can safely conclude that we have identified the gene responsible for providing surface repellence against water, and thus the fungi's ability to respond to humid environments, and in particular that the compounds responsible for providing this effect will be a compound comprising amino acids with an alternating D/L alpha architecture in the backbone.

### Example 7

### Phenotype of Aspergillus Knockout Strain

### Aim:

To determine the phenotype of the Aspergillus Fumigatus KO::Pes1 strain.

### Methods:

The methods as disclosed in examples 2 and 3 were used to study the knockout of an NRPS in Aspergillus Fumigatus. The mutants are verified by full genome sequencing.

### Results:

The analysis revealed that the Pes1 gene in Aspergillus Fumigatus encodes an NRPS as identified in previous examples.

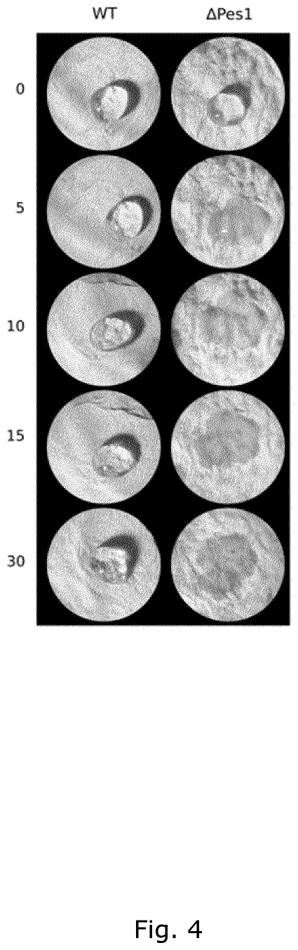

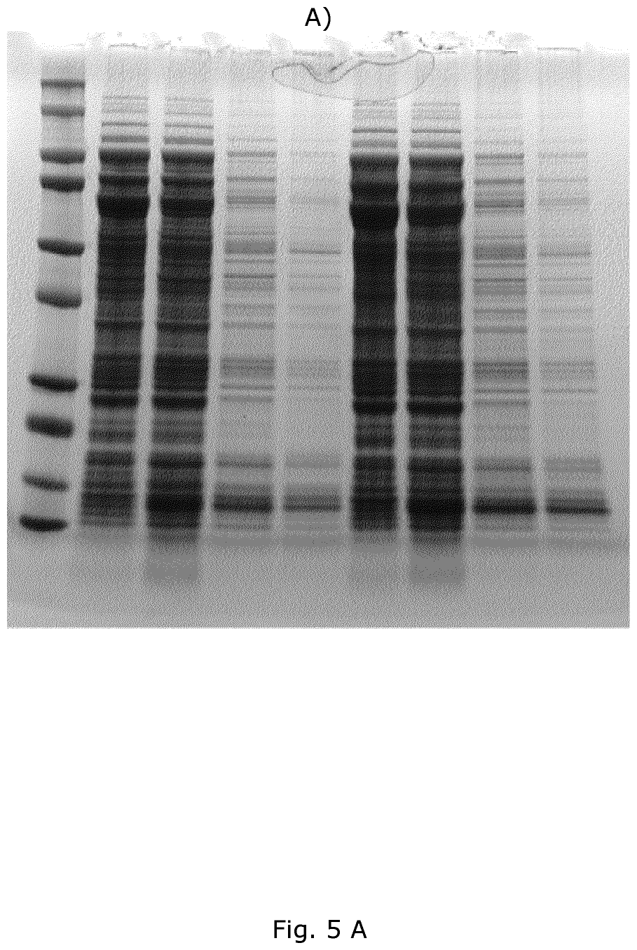

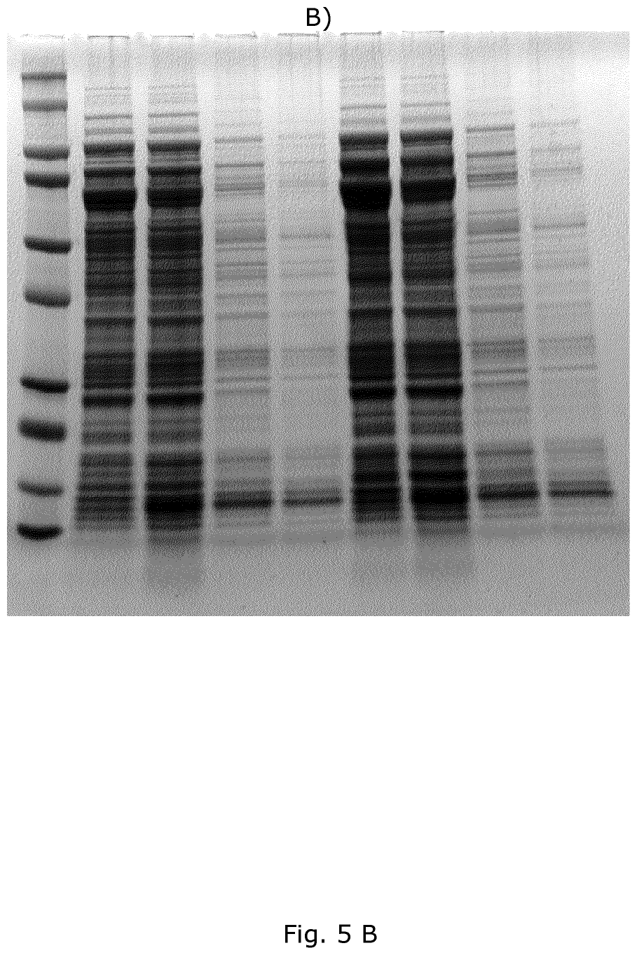

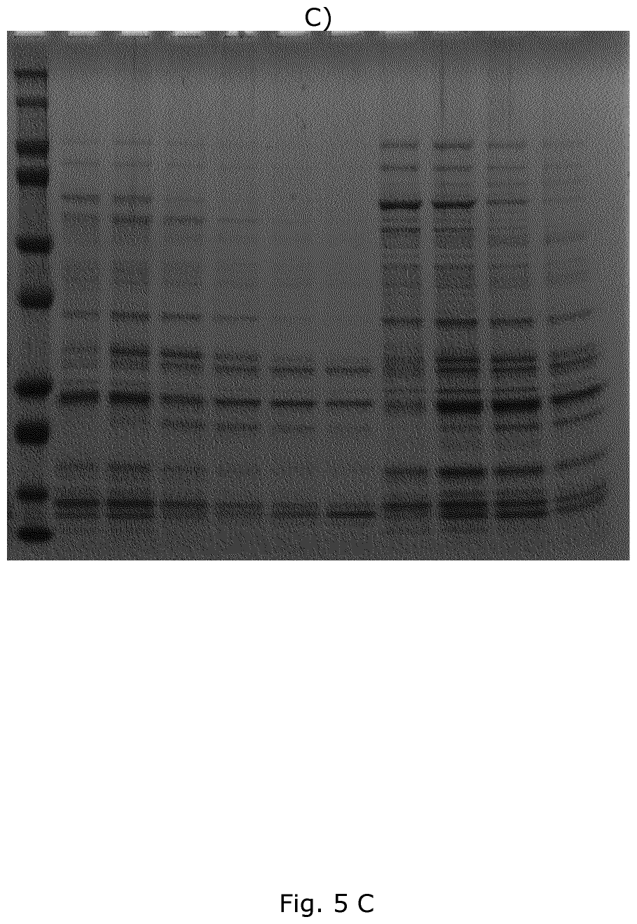

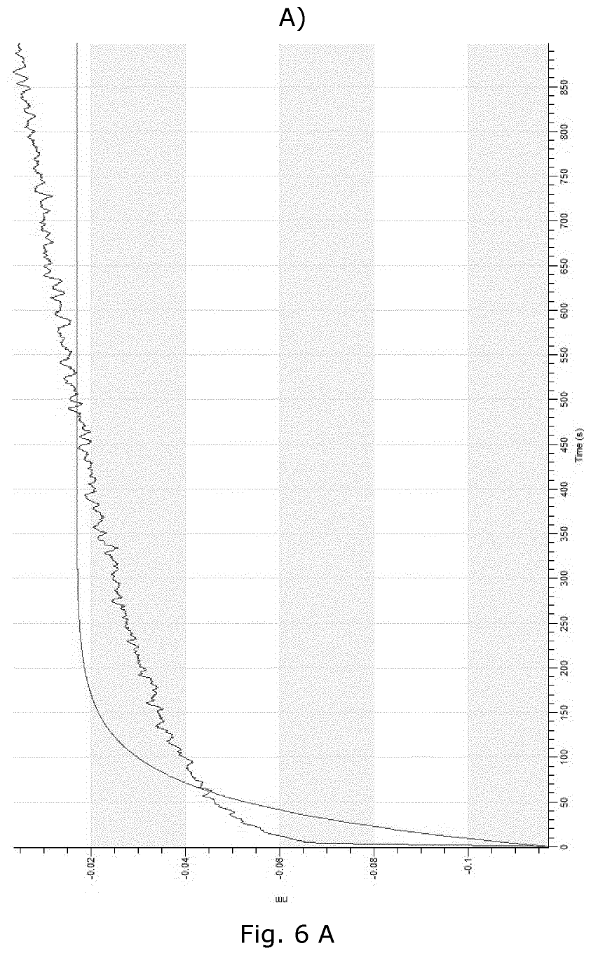

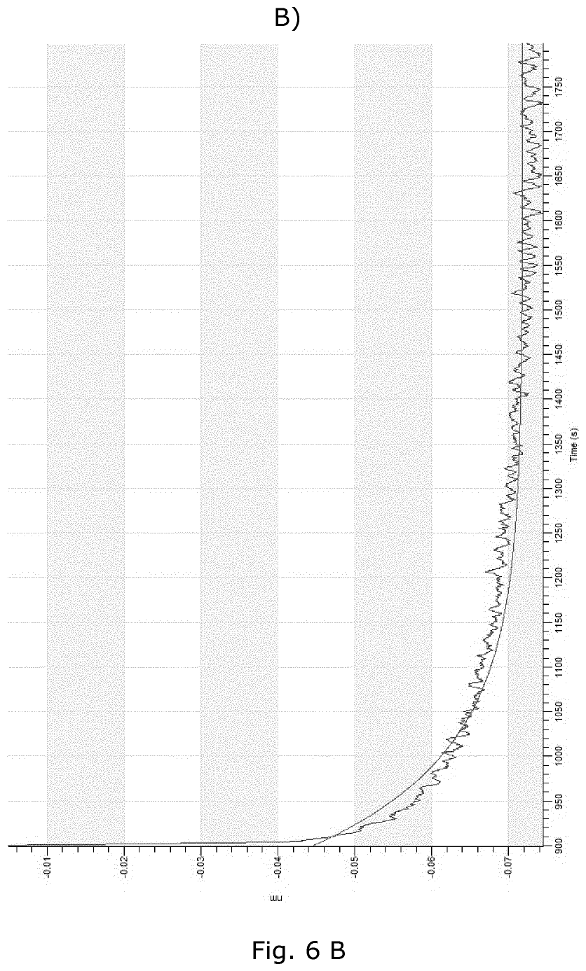

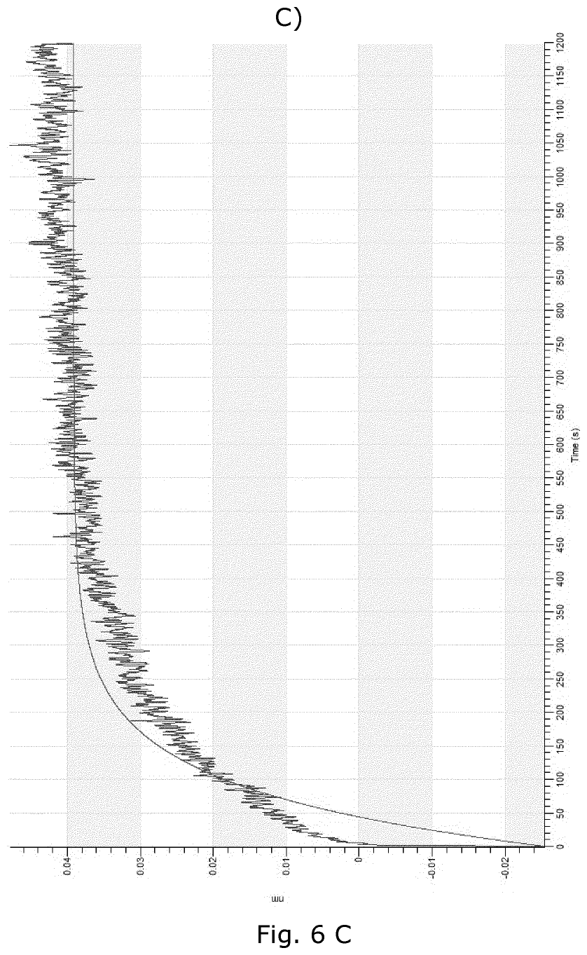

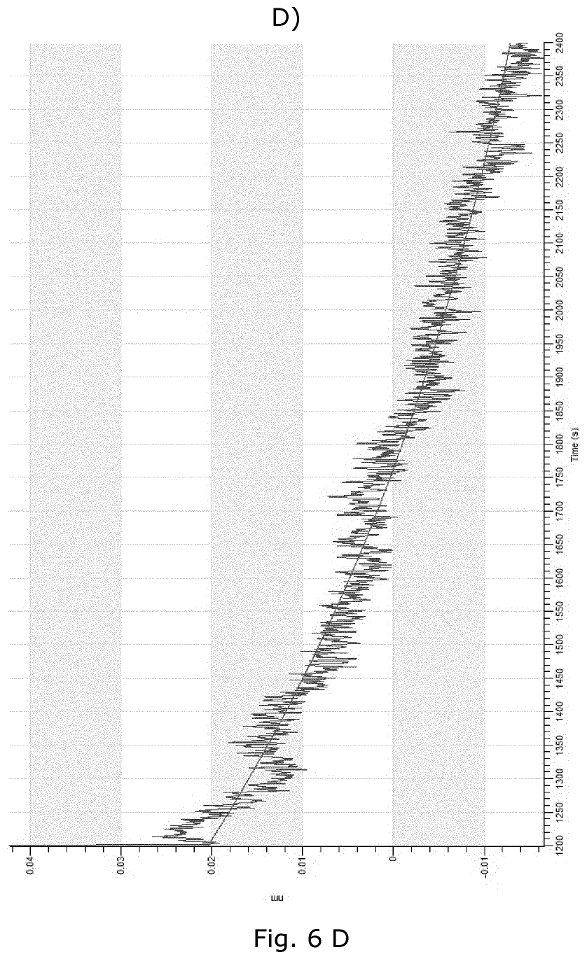

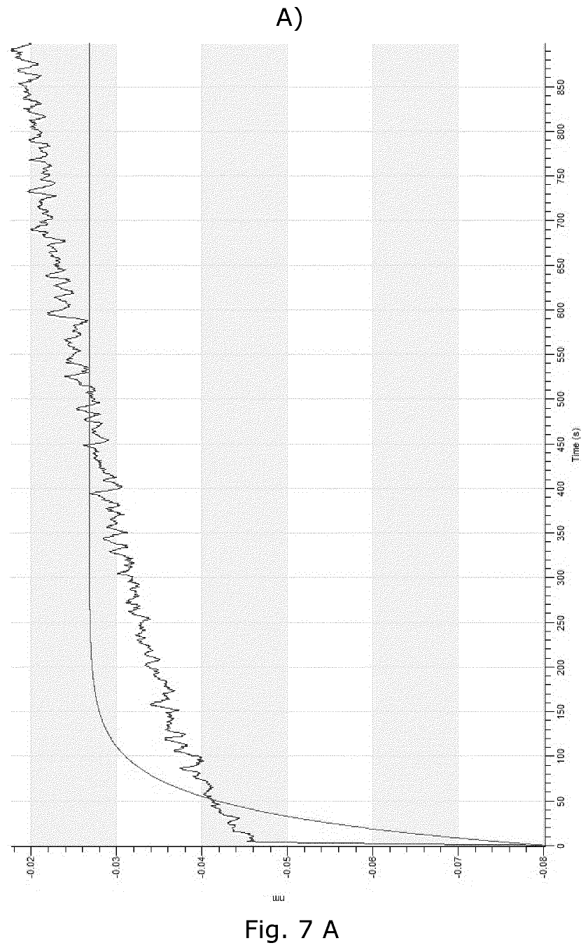

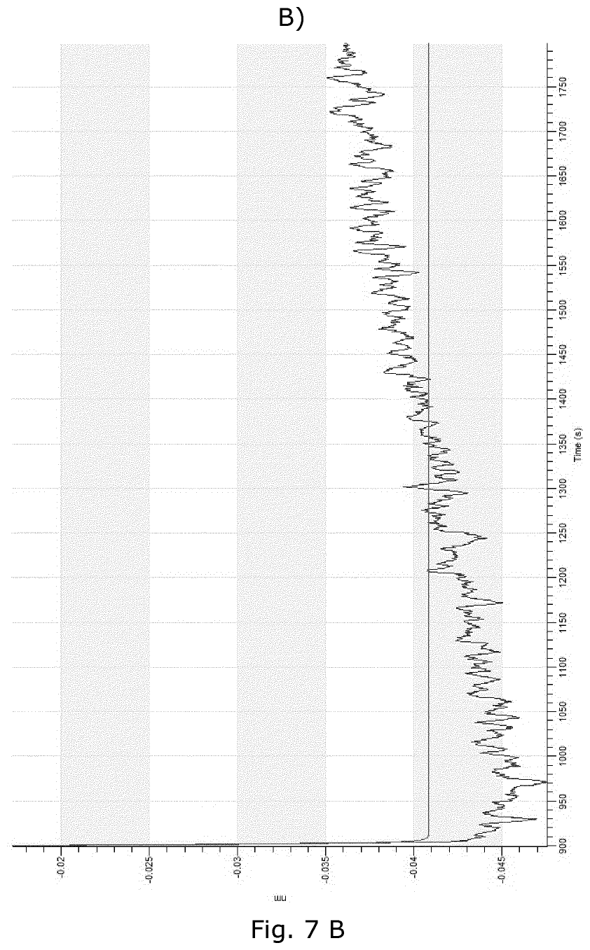

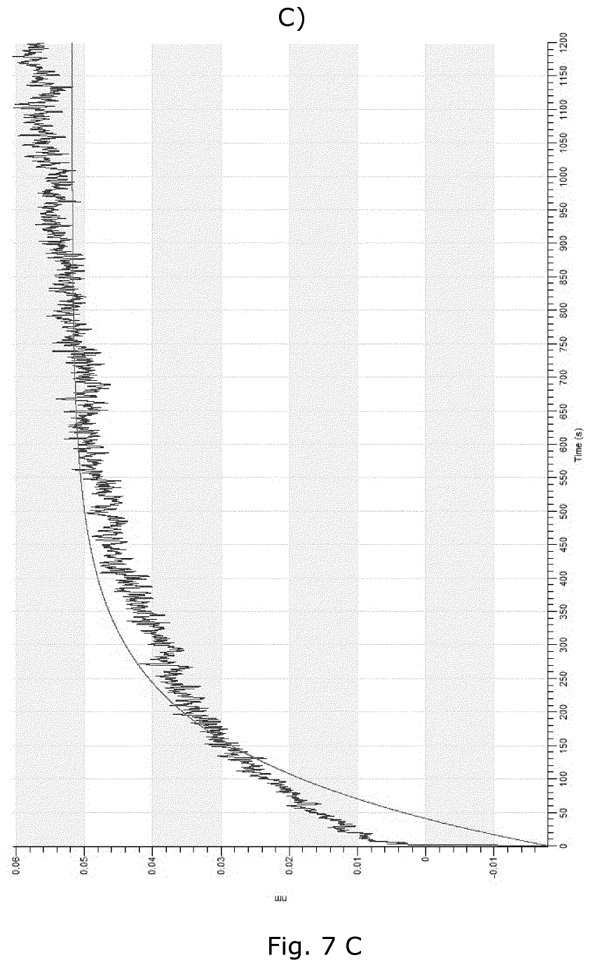

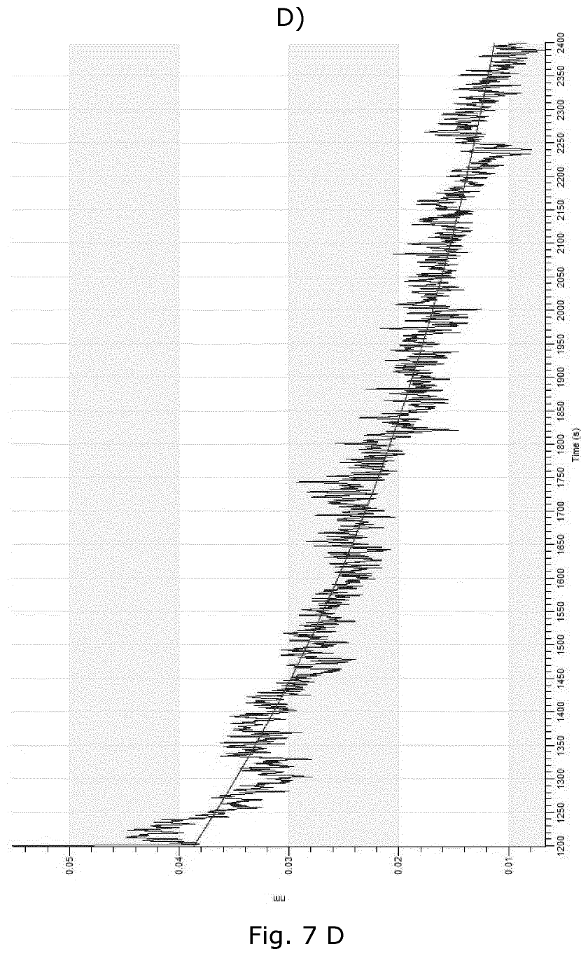

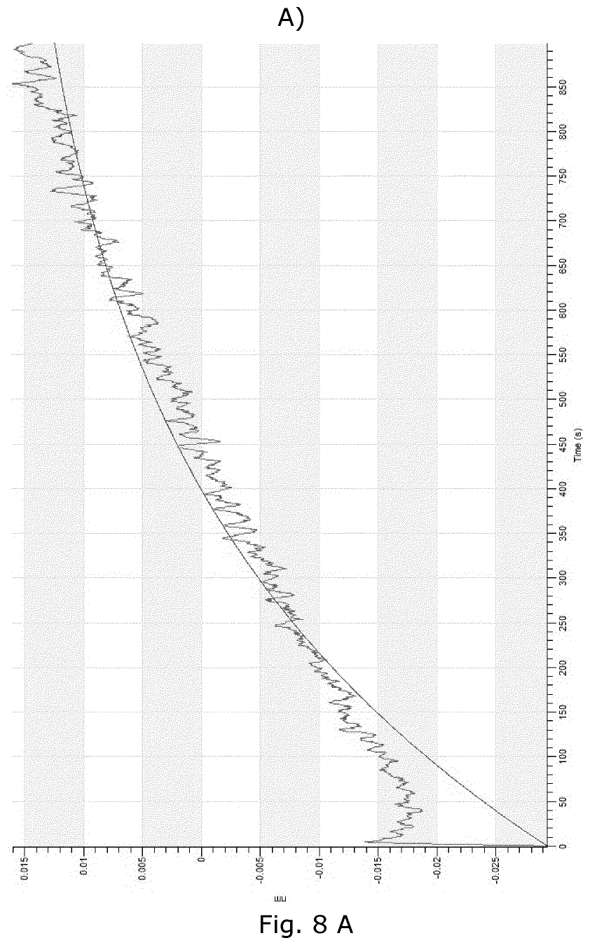

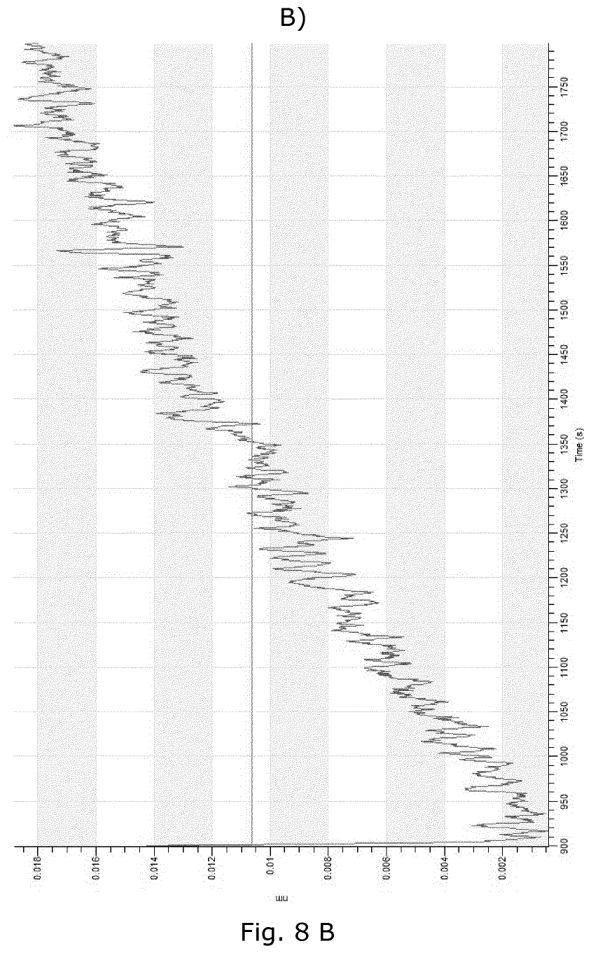

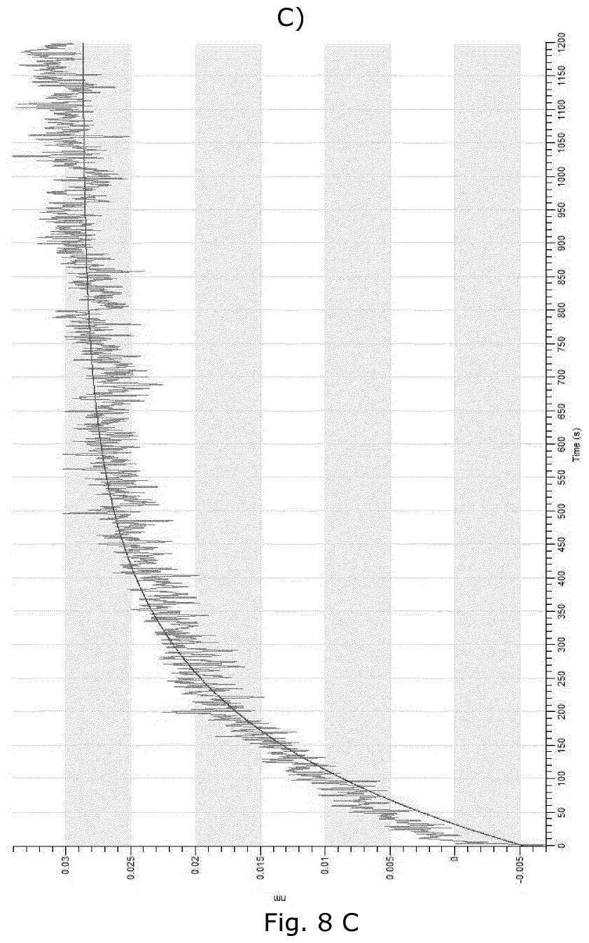

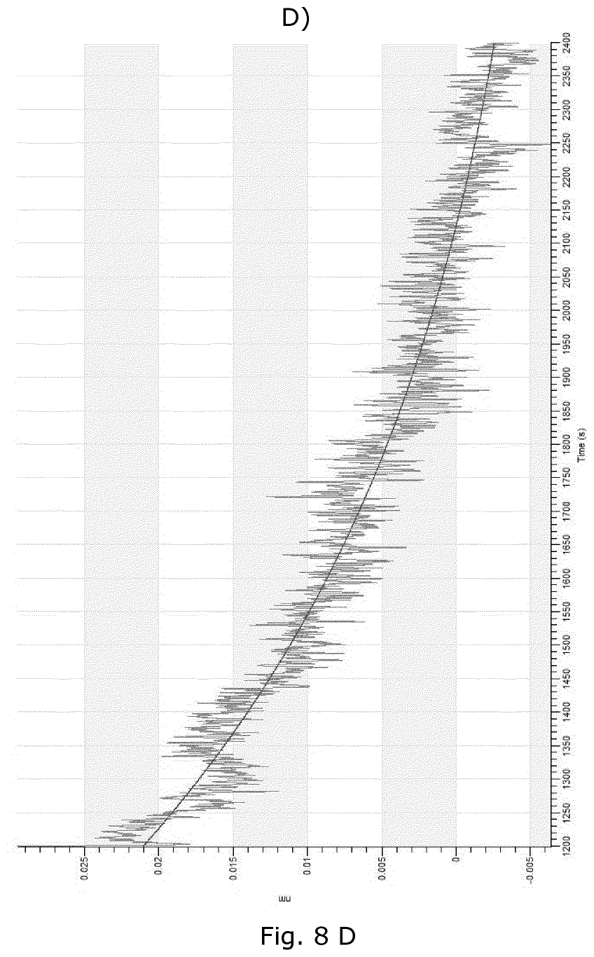

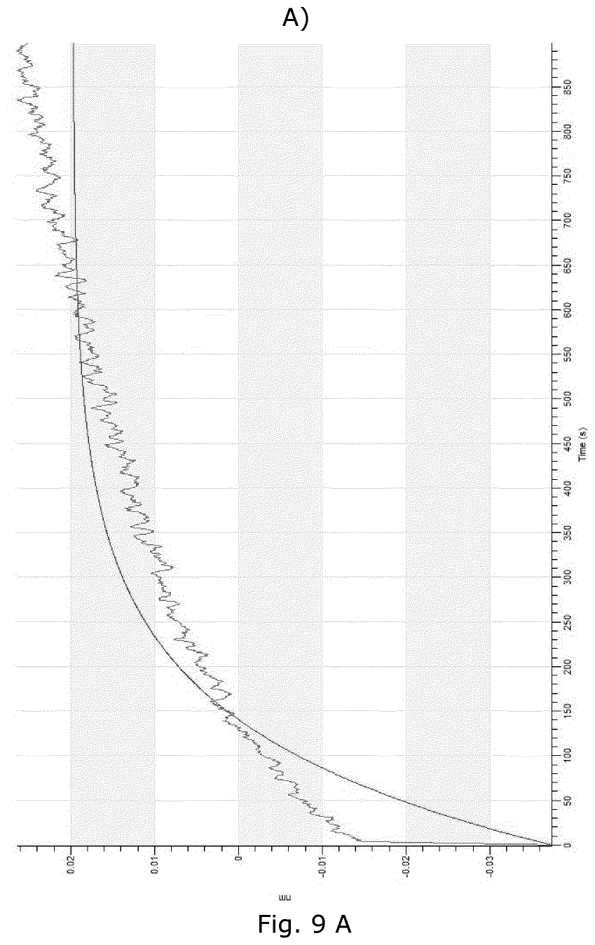

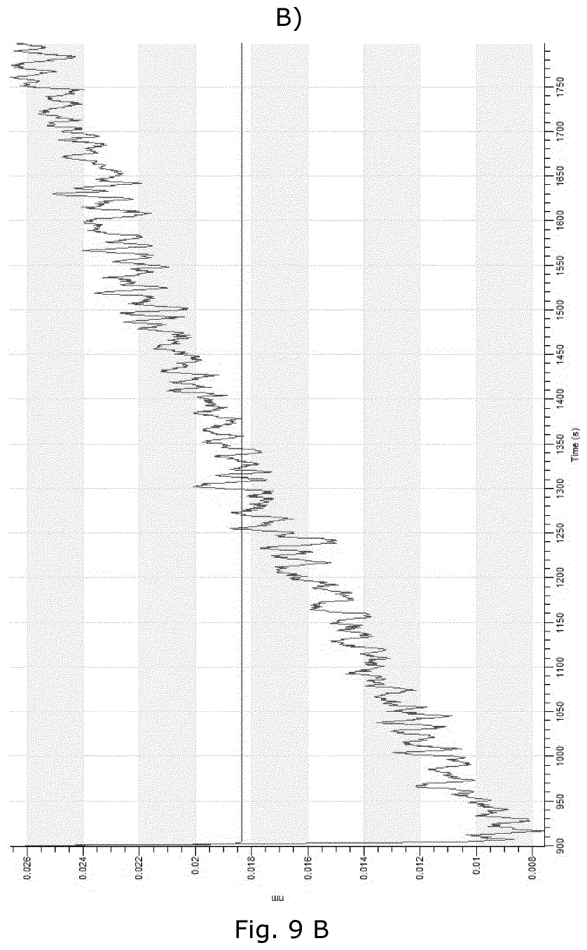

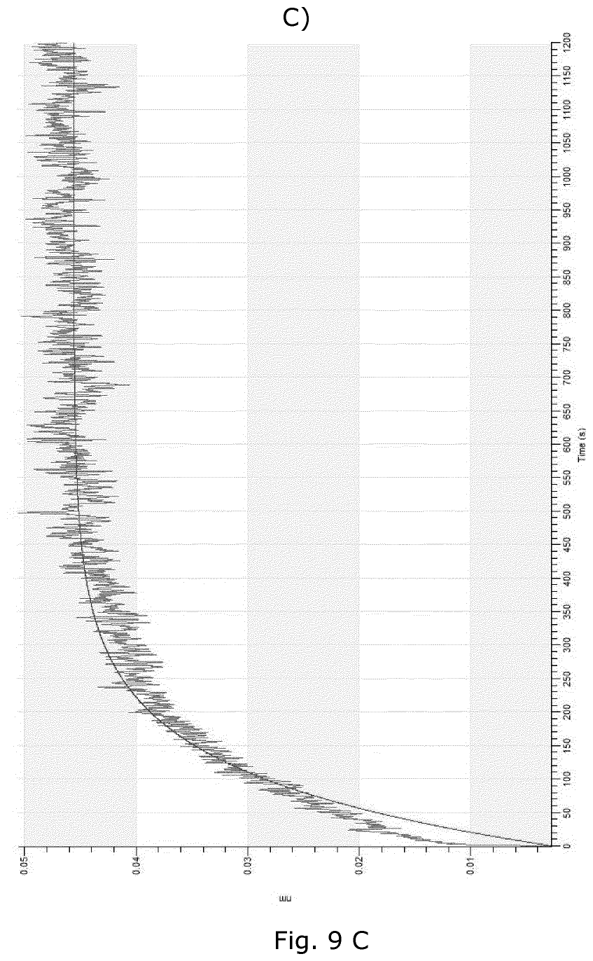

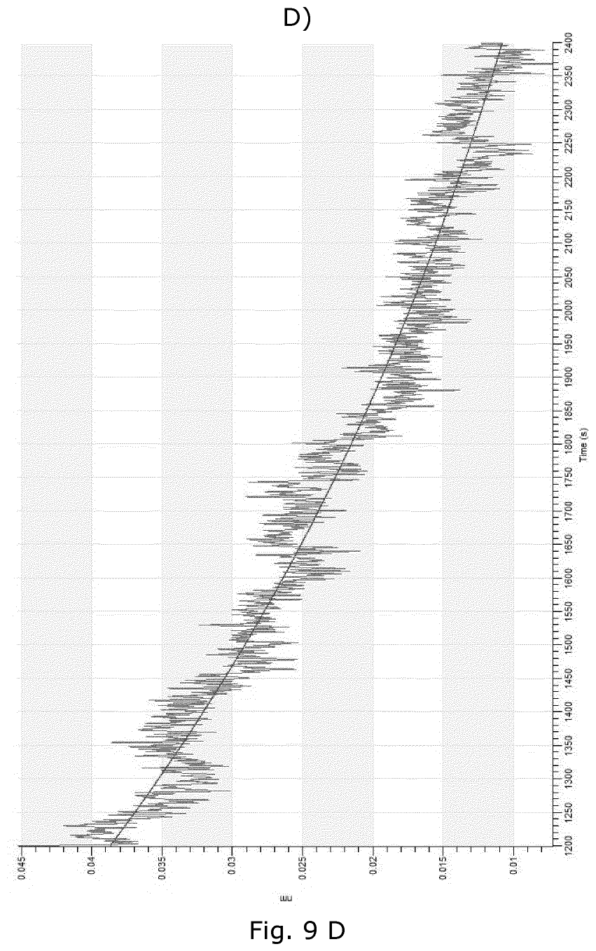

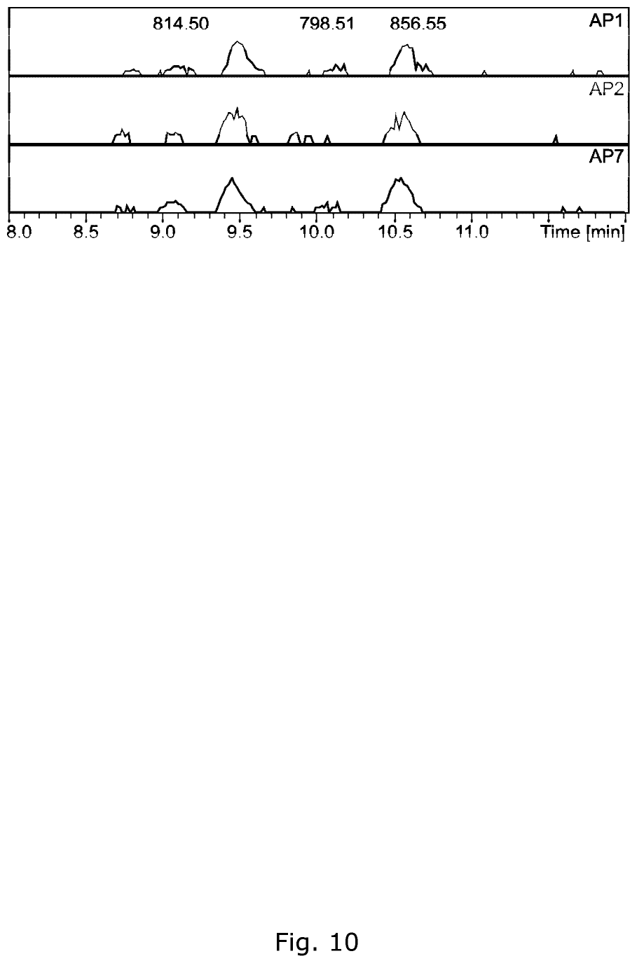

Knockout of Pes1 by CRISPR/CAS9 mutation reveals the same fungal phenotype absorbing water (see FIG. 4). A compound with the mass MW 575,27, composed off C₃₂H₃₈N₄O₆, was identified. Isolated from WT and not present in KO mutant.

### Conclusion:

Knocking out Pes1 in Aspergillus Fumigatus revealed the same phenotype (unable to repel water), and thus confirms that Pes1 is an NRPS producing a compound with the same function, and we were able to isolate a compound produced therefrom.

### Example 8

Phenotype of Trichophyton rubrum Knockout Strain

### Aim:

To determine the phenotype of the Trichophyton rubrum KO::TERG_01444 strain.

### Methods:

The methods as disclosed in examples 2 and 3 were used to study the knockout of an NRPS in Trichophyton rubrum. The mutants are verified by full genome sequencing.

### Results:

The analysis revealed that the TERG_01444 gene in Trichophyton rubrum encodes an NRPS as identified in previous examples.

We were able to knockout TERG_01444, and identify the compound produced thereform.

### Conclusion:

We were able to identify the compound produced from TERG_01444.

### Example 9

Identification of enriched single domain antibodies (sdAbs) selected on Apiospora arundinis (Isolate AAU773) and expression of selected monoclonal sdAbs.

### Aim:

The aim was to produce antibody fragments (sdAbs) allowing validation of these outside the context of the filamentous bacteriophage.

### Methods:

Based on the output from Example 5, the colonies that were formed and counted after selection, were collected by scrapping in LB medium containing 100 μg/ml Ampicillin and a 50 ml culture (LB medium containing 100 μg/ml Ampicillin) were inoculated to an OD600 of 0.1. The culture was grown at 37° C. with 200 rpm shaking until OD600 reached 1.1, then the bacteria were harvested by centrifugation at 4000 rpm for 15 minutes and phagemid were extracted from the bacterial pellet using the E.Z.N.A.® Plasmid Midi kit, from VWR (Denmark). Next generation sequencing was performed using Oxford Nanopore by the core facility at the department.

After sequencing the output sequences were trimmed based on the CDR2 and CDR3 sequences seen in the antibodies and the sequences sorted based on the number of occurrences. (SEQ ID NO: 5, SEQ ID NO: 10, SEQ ID NO: 15, SEQ ID NO: 20, SEQ ID NO: 25, SEQ ID NO: 30)

The genes encoding the 7 antibodies mostly enriched during selection were synthesized by the company GeneUniversal Inc and cloned into the plasmid pET22b (+) with a ALFA tag and HIS tag added at the 3′ end of the gene encoding the antibody fragments.

The plasmids were transformed into electrocompetent BL21(DE3) for expression. The cultures were named AP1, AP2, AP3, AP4, AP6, AP7 and AP8 and for each a single colony was used to inoculated a culture in TB medium containing a final concentration 100 μg/ml Ampicillin and 2% glucose to prevent expression in the growth phase. When the cultures reached an OD600 of roughly 1.2-1.5 before centrifuging for 30 min at 4000 g. The bacterial pellets were resuspended in fresh medium with 1 mM IPTG and expressed overnight at 20° C. Next day the bacteria were harvested at 30 min 10000 g 4° C. and then resuspended in a solubilization buffer consisting of 10 mM Na2HPO4, 1.8 mM KH2PO4, 2.7 mM KCl, 500 mM NaCl, 10 mM MgCl2, 0.75 M trehalose, 1 M glycine betaine, 10% glycerol, 0.2% Tween20 (5 ml of buffer for the roughly 1 g pellets). The resuspended bacterial pellets were sonicated at 40% duty cycle for 150 seconds at 4 sec on and 8 sec off with the MS73 probe. The lysed bacteria were then centrifuged for 30 min at 10000 g at 4° C. The supernatant was filtered (0.2 μm) and then purified on a 1 ml Histrap FF Crude column using PBS pH 8.0 as buffer with 20 mM imidazole in the wash buffer and 250 mM imidazole in the elution.

For AP1, AP2, AP3 and AP4 and isocratic elution profile was used whereas a linear gradient of imidazole from 20 mM to 250 mM were applied over 5 column volumes.

The eluted fractions (14 ml) were loaded onto a 4-20% Bis-Tris SUREPage gel (Genscript) and run at 150 V in a MOPS buffer. The gels were stained using Coomassie stain.

### Results

For all cultures soluble antibody was obtained indicated by bands of 18 kDa. Although the purity after purification indicated significant impurities, the presence of antibody was confirmed by the presence of the expected band at around 18 kDa.

### Conclusion:

Even with a relatively low purity the further steps for validation (affinity measurements and immunoprecipitation) can be performed. In both these assays the antibody fragments are mixed with fungal extracts and then captured on sensor chips or protein A Sepharose.

### Example 10

### Measuring Affinity of Antibody Fragments

### Aim:

The Aim was to establish that selected antibody fragments could capture their target antigen Apiosporin from a lysate of WT Apiospora arundinis (Isolate AAU773), whereas no antigen would be bound specifically to the antibody fragments when KO Apiospora arundinis were applied in the binding assay.

### Methods:

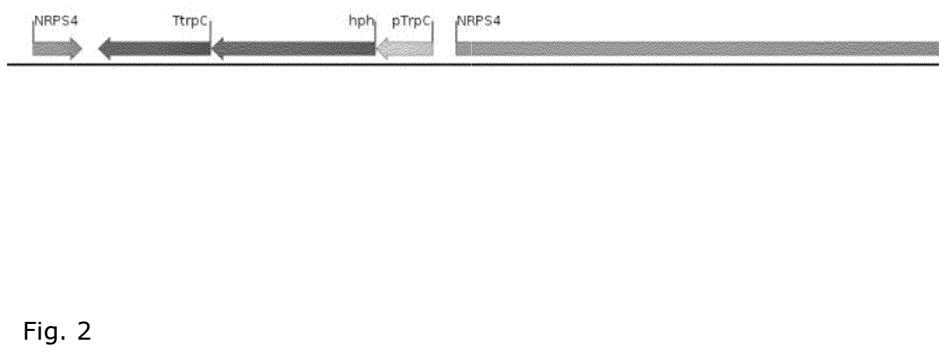

The sdAbs produced as in example 9 were applied to amine reactive biosensors from Sartorius for the Octet instrument. First the reagents were prepared according to the AR2G kit instructions. For a standard immobilization scouting assay 200 μL/well of water was added to column 1 of a 96-well plate. The 200 μL/well 1 M ethanolamine pH 8.5 were added to column 4. 200 μL/well assay running buffer into column 5. Here the provided Kinetics Buffer (dilute the 10× Kinetics Buffer 10-fold in PBS) was used. 200 μL/well of the sdAbs were added to column 6 at a concentration of 500 nM. The sdAbs were prepared to be immobilized. Protein was prepared in each of the 10 mM acetate buffers at pH 4, 5 and 6. A starting ligand concentration of at least 20 μg/mL is recommended. The ligand concentration may be decreased as low as 5 μg/mL if the immobilization time is extended; some performance loss is expected at lower ligand concentrations. Prepare 500 μL at each pH. Pipette 200 μL/well into column 3 as shown in FIG. 2.

### Results

As Apiosporin is present in an unknown concentration in the crude extracts, it is not possible to estimate a Kd from the sensorgram. Also, it is not possible to fit the raw data to a nice binding curve when a ligand concentration is not known. However, in order to allow fitting a concentration of 100 nM and 10 nM (resulting in almost identical fitting curves) of Apiosporin were inserted. The same fitting settings were used for all (data correction aligning to the association step with baseline correction to dissociation, Savitzky-Golay filtering to smoothen the curves, 1:1 model graph fitting to association and dissociation separately). The same model fit the WT data and resulted in no fit for the KO data. It thus establishes that there is a normal association/dissociation between Apiosporin and sdAbs for the WT, but no association/dissociation for the KO as expected. Thus, it is seen that the antibodies are specific against the compound produced from the NRPS.

### Conclusion

As the same model fit the WT data quite well but resulted in no fit for the KO data it established that there is a normal association/dissociation between Apiosporin and sdAbs for the WT, but no association/dissociation for the KO as expected. This Indicates that we have successfully created antibodies against the compound produced from the NRPS.

### Example 11

Capturing Apiosporin by the sdAbs and identification of the identify of Apiosporin.

### Aim:

Having established that AP1, AP2, AP3 and AP4 indicate binding to a ligand present in the WT extracts by absent in the KO, the aim is to establish that the ligand captured is indeed Apiosporin as measured by MALDI.

### Methods:

50 μl of the partial pure sdAbs prepared in example 9 and 150 μl fungal extract (resuspended in PBS with 500 mM NaCl and 0.2% Tween20) were incubated overnight at 4° C. The mixed sdAb and fungal extract were purified on a SpinTrap Protein A from Cytivia accoridng to manufactures instructions except that the elution was performed into 50% AcN 5% formic acid.

The same immune precipitations were analysed by HPLC-DAD-HRMS using a Hitachi Elite LaChrom HPLC system, equipped with a phenyl-hexyl 100 Å (150×4.6 mm Kinetex 5 μm, Phenomenex) column kept at 40° C. and coupled by a 5:95 flow splitter to a high-resolution mass spectrometer (compact qTOF, Bruker) with an electrospray source (Capilary: 4500 V; end plate offset 500 V; Dry gas 4.0 L/min, 200° C.) operated in positive mode. The injection volume was set to 30 μL and separated using a 1.2 mL/min gradient initiating at 10% solvent A (acetonitrile (HiPerSolv CHROMANORM, VWR) with 0.1% v/v formic acid (MS-grade 98%, Sigma-Aldrich)) in 90% solvent B (water (HiPerSolv CHROMANORM, VWR) with 0.1% v/v formic acid (MS-grade 98%, Sigma-Aldrich)), increasing linearly to 100% solvent A over 17 minutes and held for 6 minutes. After 0.5 minutes from sample injection, 2 μL calibrant (10 mM NaOH and 26 mM formic acid (MS-grade 98%, Sigma-Aldrich) in 1:1 water (HiPerSolv CHROMANORM, VWR): isopropanol) was injected directly to the mass analyser and used for internal spectra calibration. Mass spectrometric instrument control, data recording and data processing was performed using Compass DataAnalysis 4.2 SR2 (Bruker).

### Results:

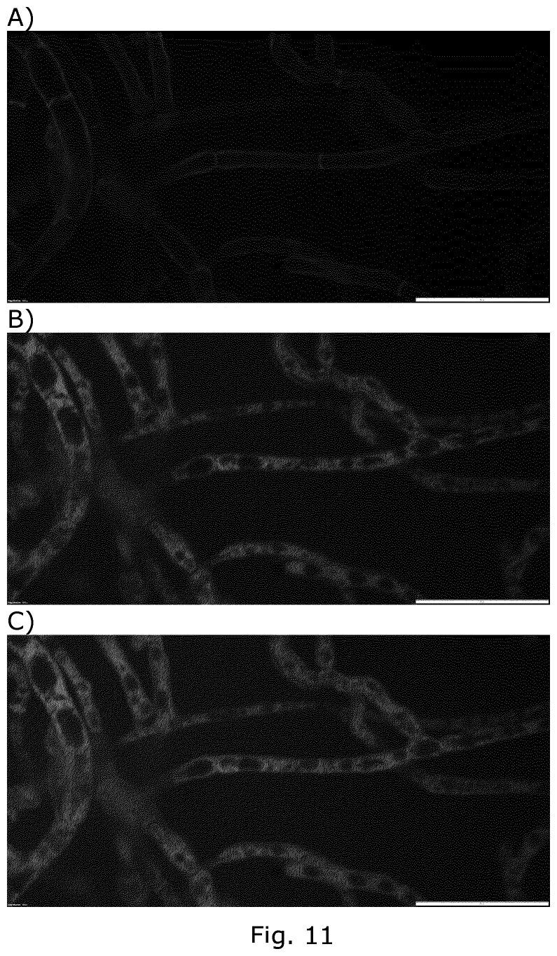

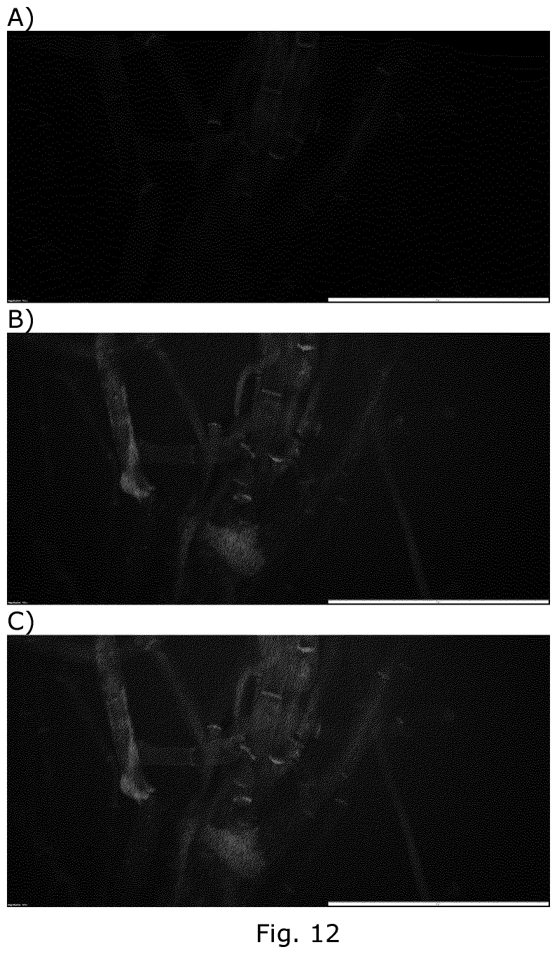

From the spectra (FIG. 11), molecules with molecular weights around the expected size of Apiosporin is present. There appears to be multiple other peaks. This could be alternative forms of Apiosporin, to which the antibodies might bind, or it could be background signals of molecules binding non-specifically to the sdAbs.

### Conclusion:

The selected antibodies are able to immunoprecipitate Apiosporin.

### Example 12

Immunoflourescent Localization of Apiosporin in Apiospora arundinis (Isolate AAU773)

### Aim

Using some of the partly purified sdAbs in example 9 immune fluorescent labeling were performed to investigate the specificity of the antibodies and the localization of Apiosporin.

### Methods:

Apiospora arundinis (Isolate AAU773) were grown in liquid culture and spotted on glass cover slips where they were immobilized using tape. The fungi were fixed by addition of 3.7% paraformaldehyde for 10 min. Then blocking with 5% skim milk in PBS before staining with the primary sdAbs (70 nM) overnight, secondary antibody (ALFA sdAb-FluoTag-X2 from Synaptic Systems, the antibody was labelled with Alexa flour 647 and used at a dilution of 1:500 for 3 h, washing 3×5 min with PBS, then staining for 2 min with calcofluor white stain (calcofluor white 1 g/l, evans blue 0.5 g/l), then wash 1×5 min with PBS.

### Results:

Proof of concept data was generated to show how the antibodies binds to fungi. By these preliminary data, staining is clearly seen in the septa, which is part of the cell wall between hyphae of WT but no staining of the septa in KO. This specifically shows that Apiosporin is located in the cell wall, and is accessible for the antibody. There appear to be significant background staining, and thus further optimization of the experimental conditions will improve the staining.

### Conclusion:

Apiosporin is located in the cell wall, and is accessible for antibody.

### Combined Conclusion on Examples

Having established that NRPS genes are present in a wide range of fungi, we were further able to knockout these genes in various species, and reproduce the same phenotype, i.e. ability to repel water.

Thus, we can safely conclude that we have identified the gene responsible for providing surface repellence against water, and thus fungi's ability to respond to humid environments, and in particular that the compounds responsible for providing this effect will be a compound comprising amino acids with an alternating D/L alpha architecture in the backbone.

Further, antibodies against these NRPS compounds can be produced and be used in therapy and detection. Additionally, this show that the NRPS-derived compounds can be bound by antigen-specific compounds, both when NRPS-derived compounds are bound in a cell membrane, however also when present in solution.
Secondly, Apiosporin, and NRPS produced compounds in general is present on the extracellular surface of Apiospora arundinis showing that the epitopes will be accesible to the immune system, and that vaccines can be generated by using NRPS-derived compounds.

Sequence listing and-overview

SEQ ID NO: 1-Apiospora arundinis (AAU773), NRPS4 gene sequence
See sequence listing for complete sequence
SEQ ID NO: 2-Apiospora arundinis (AAU773), NRPS4 CDS sequence
See sequence listing for complete sequence
SEQ ID NO: 3-Apiospora arunidinis (AAU773), NRPS4 mRNA
See sequence listing for complete sequence
SEQ ID NO: 4-Apiospora arundinis (AAU773), NRPS4 amino acid sequence
See sequence listing for complete sequence
SEQ ID NO: 5-AP: 1-DNA sequence
GAAGTTCAGCTGCTGGAAAGCGGCGGCGGCCTGGTTCAGCCGGGCGGGTCCCTGCGCC
TGAGCTGCGCGGCTAGCGGCTTTCGGGATTCCGATGAAGATATGGGCTGGGTGCGCCA
GGCACCTGGTAAAGGCCTGGAATGGGTGAGCTCCATTTACTCTTACAACGGGTCTACCTA
TTATGCAGATAGCGTGAAAGGCCGCTTTACCATCTCGAGAGATAATTCGAAAAACACCCT
GTATCTGCAAATGAACAGCCTGCGTGCGGAAGATACTGCAGTTTATTATTGCGCGAGCGA
CCGTGGTGGTTACAACTCTTTCGACTACTGGGGCCAGGGTACCCTGGTGACTGTGAGCA
GCGCGGCCGCA
SEQ ID NO: 6-AP: 1-Protein sequence
EVQLLESGGGLVQPGGSLRLSCAASGFRDSDEDMGWVRQAPGKGLEWVSSIYSYNGSTYY
ADSVKGRFTISRDNSKNTLYLQMNSLRAEDTAVYYCASDRGGYNSFDYWGQGTLVTVSSA
AA
SEQ ID NO: 7-AP: 1 CDR1-Protein sequence
GFRDSDEDMG
SEQ ID NO: 8-AP: 1 CDR2-Protein sequence
IYSYNGSTYYADSVKG
SEQ ID NO: 9-AP: 1 CDR3-Protein sequence
DRGGYNSFDY
SEQ ID NO: 10-AP: 2-DNA sequence
GAAGTTCAGCTGCTGGAAAGCGGCGGCGGCCTGGTTCAGCCGGGCGGGTCCCTGCGCC
TGAGCTGCGCGGCTAGCGGCTTTCGGGATTCCGATGAAGATATGGGCTGGGTGCGCCA
GGCACCTGGTAAAGGCCTGGAATGGGTGAGCTCCATTGAAACTCCGGACGGGTCTACCT
ATTATGCAGATAGCGTGAAAGGCCGCTTTACCATCTCGAGAGATAATTCGAAAAACACCC
TGTATCTGCAAATGAACAGCCTGCGTGCGGAAGATACTGCAGTTTATTATTGCGCGAGCC
AGCCGGTTTACTCTTACCATTTCGACTACTGGGGCCAGGGTACCCTGGTGACTGTGAGCA
GCGCGGCCGCA
SEQ ID NO: 11-AP: 2-Protein sequence
EVQLLESGGGLVQPGGSLRLSCAASGFRDSDEDMGWVRQAPGKGLEWVSSIETPDGSTYY
ADSVKGRFTISRDNSKNTLYLQMNSLRAEDTAVYYCASQPVYSYHFDYWGQGTLVTVSSAA
A
SEQ ID NO: 12-AP: 2 CDR1-Protein sequence
GFRDSDEDMG
SEQ ID NO: 13-AP: 2 CDR2-Protein sequence
IETPDGSTYYADSVKG
SEQ ID NO: 14-AP: 2 CDR3-Protein sequence
QPVYSYHFDY
SEQ ID NO: 15-AP: 3-DNA sequence
GAAGTTCAGCTGCTGGAAAGCGGCGGCGGCCTGGTTCAGCCGGGCGGGTCCCTGCGCC
TGAGCTGCGCGGCTAGCGGCTTTCGGGATTCCGATGAAGATATGGGCTGGGTGCGCCA
GGCACCTGGTAAAGGCCTGGAATGGGTGAGCTCCATTGGTGGTTACAACGGGTCTACCT
ATTATGCAGATAGCGTGAAAGGCCGCTTTACCATCTCGAGAGATAATTCGAAAAACACCC
TGTATCTGCAAATGAACAGCCTGCGTGCGGAAGATACTGCAGTTTATTATTGCGCGAGCG
AAGGTTACTACACTTCTGCTTTCGACTACTGGGGCCAGGGTACCCTGGTGACTGTGAGCA
GCGCGGCCGCA
SEQ ID NO: 16-AP: 3-Protein sequence
EVQLLESGGGLVQPGGSLRLSCAASGFRDSDEDMGWVRQAPGKGLEWVSSIGGYNGSTY
YADSVKGRFTISRDNSKNTLYLQMNSLRAEDTAVYYCASEGYYTSAFDYWGQGTLVTVSSA
AA
SEQ ID NO: 17-AP: 3 CDR1-Protein sequence
GFRDSDEDMG
SEQ ID NO: 18-AP: 3 CDR2-Protein sequence
IGGYNGSTYYADSVKG
SEQ ID NO: 19-AP: 3 CDR3-Protein sequence
EGYYTSAFDY
SEQ ID NO: 20-AP: 4-DNA sequence
GAAGTTCAGCTGCTGGAAAGCGGCGGCGGCCTGGTTCAGCCGGGCGGGTCCCTGCGCC
TGAGCTGCGCGGCTAGCGGCTTTCGGGATTCCGATGAAGATATGGGCTGGGTGCGCCA
GGCACCTGGTAAAGGCCTGGAATGGGTGAGCTCCATTGACGCTGAAGACGGGTCTACCT
ATTATGCAGATAGCGTGAAAGGCCGCTTTACCATCTCGAGAGATAATTCGAAAAACACCC
TGTATCTGCAAATGAACAGCCTGCGTGCGGAAGATACTGCAGTTTATTATTGCGCGAGCG
ACTCTGGTGACTGGTTCTCTTTCGACTACTGGGGCCAGGGTACCCTGGTGACTGTGAGC
AGCGCGGCCGCA
SEQ ID NO: 21-AP: 4-Protein sequence
EVQLLESGGGLVQPGGSLRLSCAASGFRDSDEDMGWVRQAPGKGLEWVSSIDAEDGSTYY
ADSVKGRFTISRDNSKNTLYLQMNSLRAEDTAVYYCASDSGDWFSFDYWGQGTLVTVSSA
AA
SEQ ID NO: 22-AP: 4 CDR1-Protein sequence
GFRDSDEDMG
SEQ ID NO: 23-AP: 4 CDR2-Protein sequence
IDAEDGSTYYADSVKG
SEQ ID NO: 24-AP: 4 CDR3-Protein sequence
DSGDWFSFDY
SEQ ID NO: 25-AP: 6-DNA sequence
GAAGTTCAGCTGCTGGAAAGCGGCGGCGGCCTGGTTCAGCCGGGCGGGTCCCTGCGCC
TGAGCTGCGCGGCTAGCGGCTTTCGGGATTCCGATGAAGATATGGGCTGGGTGCGCCA
GGCACCTGGTAAAGGCCTGGAATGGGTGAGCTCCATTGACTCTCCGAACGGGTCTACCT
ATTATGCAGATAGCGTGAAAGGCCGCTTTACCATCTCGAGAGATAATTCGAAAAACACCC
TGTATCTGCAAATGAACAGCCTGCGTGCGGAAGATACTGCAGTTTATTATTGCGCGAGCT
ACGACGGTTACTACTGGTGGTTCGACTACTGGGGCCAGGGTACCCTGGTGACTGTGAGC
AGCGCGGCCGCA
SEQ ID NO: 26-AP: 6-Protein sequence
EVQLLESGGGLVQPGGSLRLSCAASGFRDSDEDMGWVRQAPGKGLEWVSSIDSPNGSTYY
ADSVKGRFTISRDNSKNTLYLQMNSLRAEDTAVYYCASYDGYYWWFDYWGQGTLVTVSSA
AA
SEQ ID NO: 27-AP: 6 CDR1-Protein sequence
GFRDSDEDMG
SEQ ID NO: 28-AP: 6 CDR2-Protein sequence
IDSPNGSTYYADSVKG
SEQ ID NO: 29-AP: 6 CDR3-Protein sequence
YDGYYWWFDY
SEQ ID NO: 30-AP: 7-DNA sequence
GAAGTTCAGCTGCTGGAAAGCGGCGGCGGCCTGGTTCAGCCGGGCGGGTCCCTGCGCC
TGAGCTGCGCGGCTAGCGGCTTTCGGGATTCCGATGAAGATATGGGCTGGGTGCGCCA
GGCACCTGGTAAAGGCCTGGAATGGGTGAGCTCCATTGAAGGTCCGTCTGGGTCTACCT
ATTATGCAGATAGCGTGAAAGGCCGCTTTACCATCTCGAGAGATAATTCGAAAAACACCC
TGTATCTGCAAATGAACAGCCTGCGTGCGGAAGATACTGCAGTTTATTATTGCGCGAGCG
TTACTTGGTGGACTCATCCGTTCGACTACTGGGGCCAGGGTACCCTGGTGACTGTGAGC
AGCGCGGCCGCA
SEQ ID NO: 31-AP: 7-Protein sequence
EVQLLESGGGLVQPGGSLRLSCAASGFRDSDEDMGWVRQAPGKGLEWVSSIEGPSGSTYY
ADSVKGRFTISRDNSKNTLYLQMNSLRAEDTAVYYCASVTWWTHPFDYWGQGTLVTVSSA
AA
SEQ ID NO: 32-AP: 7 CDR1-Protein sequence
GFRDSDEDMG
SEQ ID NO: 33-AP: 7 CDR2-Protein sequence
IEGPSGSTYYADSVKG
SEQ ID NO: 34-AP: 7 CDR3-Protein sequence
VTWWTHPFDY
SEQ ID NO: 35-AP: 8-DNA sequence
GAAGTTCAGCTGCTGGAAAGCGGCGGCGGCCTGGTTCAGCCGGGCGGGTCCCTGCGCC
TGAGCTGCGCGGCTAGCGGCTTTCGGGATTCCGATGAAGATATGGGCTGGGTGCGCCA
GGCACCTGGTAAAGGCCTGGAATGGGTGAGCTCCATTGGTACTTACGACGGGTCTACCT
ATTATGCAGATAGCGTGAAAGGCCGCTTTACCATCTCGAGAGATAATTCGAAAAACACCC
TGTATCTGCAAATGAACAGCCTGCGTGCGGAAGATACTGCAGTTTATTATTGCGCGAGCT
ACCCGCAGGCTAACGTTGCTTTCGACTACTGGGGCCAGGGTACCCTGGTGACTGTGAGC
AGCGCGGCCGCA
SEQ ID NO: 36-AP: 8-Protein sequence
EVQLLESGGGLVQPGGSLRLSCAASGFRDSDEDMGWVRQAPGKGLEWVSSIGTYDGSTYY
ADSVKGRFTISRDNSKNTLYLQMNSLRAEDTAVYYCASYPQANVAFDYWGQGTLVTVSSAA
A
SEQ ID NO: 37-AP: 8 CDR1-Protein sequence
GFRDSDEDMG
SEQ ID NO: 38-AP: 8 CDR2-Protein sequence
IGTYDGSTYYADSVKG
SEQ ID NO: 39-AP: 8 CDR3-Protein sequence
YPQANVAFDY
SEQ ID NO: 40-PES1, AFUA_1G10380-DNA sequence
See sequence listing for complete sequence
SEQ ID NO: 41-XP_752404.1|: 1-6269 nonribosomal peptide synthase Pes1-
Protein sequence
See sequence listing for complete sequence

### Items

1. A method of analysing and/or identifying one or more compounds produced by a fungus-derived Non-Ribosomal Peptide Synthetase (NRPS), wherein the method comprises:
   a. providing a first fungus, such as Apiospora, and a second fungus such as the same species as the first fungus, such as the same strain as the first fungus, such as Apiospora;
   b. genetically modifying the expression of the NRPS in the first fungus, such as knocking out or overexpressing the NRPS gene;
   c. growing the first fungus and the second fungus under conditions suitable for the production of compounds, such as compounds of NRPS;
   d. recovering extracts from the first fungus and the second fungus;
   e. analysing the extracts; such as by LC-MS and/or NMR spectroscopy; and
   f. comparing the analyses of the extracts between the first fungus and the second fungus, thereby identifying compounds of the NRPS.
2. The method according to item 1, wherein the knocking out is achieved by gene editing, such as site-directed mutagenesis, such as the use of CRISPR/Cas9.
3. The method according to any of the preceding items, wherein overexpression is achieved by inserting a promoter upstream of the NRPS gene and/or upstream of the NRPS cluster specific transcription factor, such as a constitutively active promoter.
4. The method according to any of the preceding items, wherein the NRPS gene comprises a domain architecture wherein an epimerization (E) domain is present in alternating domains.
5. The method according to any of the preceding items, wherein the fungus is Apiospora.
6. The method according to any of the preceding items, wherein the Apiospora is Apiospora arundinis.
7. The method according to any of the preceding items, wherein the Apiospora arundinis is Apiospora arundinis AAU773.
8. The method according to any of the preceding items, wherein the fungus is not Neurospora crassa.
9. The method according to any of the preceding items, wherein the fungus is able to repel water, such as in a water droplet retention time study.
10. The method according to any of the preceding items, wherein the fungus is a mammalian disease-relevant fungus, such as a human disease-relevant fungus, such as a fungus selected from Aspergillus, Trichophyton, Candida, and Fusarium.
11. The method according to any of the preceding items, wherein the NRPS is encoded by the gene according to SEQ ID NO: 1, or a degenerate sequence thereof.
12. The method according to any of the preceding items, wherein the NRPS is encoded by the cDNA according to SEQ ID NO: 2, or a degenerate sequence thereof.
13. The method according to any of the preceding items, wherein the NRPS is the protein according to SEQ ID NO: 4, or a variant thereof having at least 80% sequence identity.
14. The method according to any of the preceding items, wherein the compound is a secondary metabolite.
15. A compound identified by the method according to any of the preceding items.
16. A compound produced from the Non-Ribosomal Peptide Synthetase (NRPS).
17. The compound according to any of items 15-16, wherein the compound is a secondary metabolite.
18. A compound, such as a secondary metabolite produced from a Non-Ribosomal Peptide Synthetase (NRPS) of a fungus, such as the Non-Ribosomal Peptide Synthetase 4 (NRPS4) of Apiospora.
19. The compound according to any of items 15-18, wherein the compound comprises amino acids, such as proteinogenic amino acids and/or non-proteinogenic amino acids.
20. The compound according to any of items 15-19, wherein the compound is further modified by at least one additional enzyme.
21. The compound according to any of items 15-20, wherein the compound comprises a mixture of proteinogenic amino acids and non-proteinogenic amino.
22. The compound according to any of items 19-21, wherein at least two of the amino acids, such as all amino acids, are coupled by peptide bonds formed between the α-nitrogen atom of one amino acid and the carbonyl carbon of a second amino acid.
23. The compound according to any of items 15-22, wherein the compound is a peptide.
24. The compound according to any of items 19-23, wherein the amino acids in the compound are positioned in a stereochemistry according to the general formula (L-D)ⁿ and/or (D-L)ⁿ, wherein n is an integer from 3-10, such as any of 3, 4, 5, 6, 7, 8, 9, or 10.
25. The compound according to any of items 19-24, wherein each consecutive amino acid has sidechains in an opposing enantiomer in relation to the previous amino acid.
26. The compound according to any of items 19-25, wherein the compound comprises a D/L alpha architecture in the backbone.
27. The compound according to any of items 15-26, wherein the compound comprises 3-10 amino acids, such as any of 3 amino acids, such as 4 amino acids, such as 5 amino acids, such as 6 amino acids, such as 7 amino acids, such as 8 amino acids, such as 9 amino acids, or such as 10 amino acids, preferably such as 7 amino acids.
28. The compound according to any of items 15-27, wherein the compound is cyclic.
29. The compound according to any of items 15-27, wherein the compound is linear.
30. The compound according to any of items 15-29, wherein the compound is a compound represented by Formula I-VI selected from:

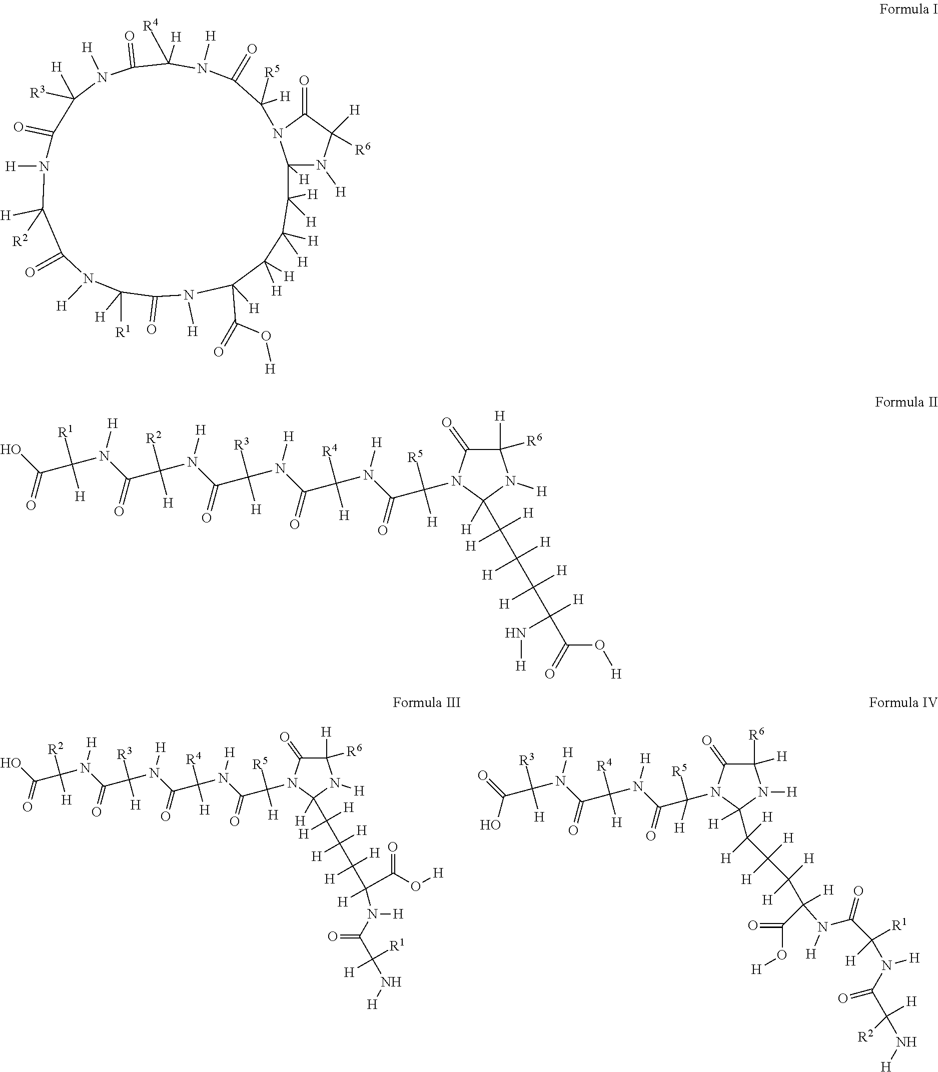

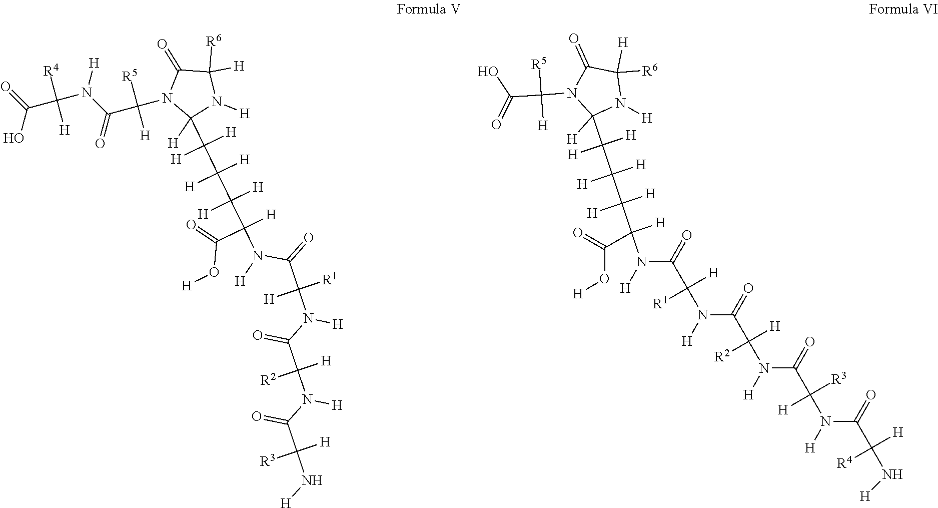

wherein R¹ is L-Tyrosine or D-Tyrosine; R² is L-Leucine or D-Leucine; R³ is L-Alanine, D-Alanine, L-Leucine or D-Leucine; R⁴ is L-Leucine or D-Leucine; R⁵ is L-Leucine or D-Leucine; and R⁶ is L-Leucine or D-Leucine, and the Lysine is a L-Lysine or a D-Lysine, with the proviso that when R¹, R³, R⁵ are L-enantiomers, R², R⁴, and R⁶ are D-enantiomers, or when R¹, R³, R⁵ are D-enantiomers, R², R⁴, and R⁶ are L-enantiomers.
31. The compound according to any of items 15-30, wherein the compound is a salt thereof, such as a pharmaceutically acceptable salt thereof.
32. The compound according to any of items 15-31, wherein the Apiospora is Apiospora arundinis.
33. The compound according to item 32, wherein the Apiospora arundinis is
34. The compound according to any of items 15-33, wherein the NRPS4 is encoded by the gene according to SEQ ID NO: 1, or a degenerate sequence thereof.
35. The method according to any of items 15-33, wherein the NRPS4 is encoded by the cDNA according to SEQ ID NO: 2, or a degenerate sequence thereof.
36. The compound according to any of items 15-33, wherein the NRPS4 is the protein according to SEQ ID NO: 4, or a variant thereof having at least 80% sequence identity.
37. The compound according to any of items 15-36, wherein the compound is coupled to an adjuvant, such as a toxin.
38. The compound according to any of items 15-37 for use as a vaccine.
39. The compound for use according to any of the preceding items, wherein the vaccine is a vaccine against a fungus infection.
40. The compound for use according to any of items 38-39, wherein the fungus infection is an infection with the fungus of which the compound is originally derived, such as where the NRPS is present in the genome of the fungus of which the compound is originally derived.
41. Use of the compound according to any of items 15-36 as a surfactant.
42. An antigen-binding molecule having affinity against the compound according to any of items 15-37, such as an antibody, or an antigen-binding fragment thereof.
43. An antigen-binding molecule having affinity against the extracellular surface of Apiospora, such as an antibody, or an antigen-binding fragment thereof.
44. The antigen-binding molecule according to any items 42-43, wherein the extracellular surface of Apiospora is a compound according the any of the preceding items, present on the surface of the fungus.
45. The antigen-binding molecule according to any items 42-44, wherein the antigen-binding molecule can bind to a solubilized form of the compound, such as the not being bound to a cellular membrane, such as the membrane of a fungus.
46. The antigen-binding molecule according to any items 42-45, wherein the antigen-binding molecule is a molecule selected from the group consisting of an antibody, a scFv, a single domain antibody (sdAb) or nanobody, a VHH, an isolated single variable domain, an affibody, a DARPin, a monobody, an anticalin, an affilin, an affimer type 1 molecule, an affimer type 2 molecule, an affitin, an alphabody, an anticalin, an avimer, a fynomer, a kunitz domain peptide, a nanoclamp, and an aptamer.
47. The antigen-binding molecule according to any items 42-46, wherein the antigen-binding molecule comprises:
   a heavy chain variable region comprising a CDR 1 region, a CDR 2 region, and a CDR 3 region; and
   a light chain variable region comprising a CDR 1 region, a CDR 2 region, and a CDR 3 region.
48. Use of the antigen-binding molecule according to any items 42-47 in a method of detecting Apiospora, such as an in vitro method.
49. The antigen-binding molecule according to any of items 42-47 for use as a medicament.
50. The antigen-binding molecule according to any of items 42-47 for use in the treatment of an infection with a mammalian disease-relevant fungus, such as a human disease-relevant fungus, such as a fungus selected from Aspergillus, Trichophyton, Candida, and Fusarium.
51. The antigen-binding molecule according to any of items 42-47, wherein the antigen-binding molecule is an antibody-drug conjugate, such as coupled to a toxin or a medicament.
52. The antigen-binding molecule according to any of items 42-47 or item 51, wherein the antigen-binding molecule is coupled to a toxin.
53. Use of the antigen-binding molecule according to any of items 42-47 or any of items 51-52 in the treatment of a non-human infection, such as an infection on trees, such as a tree selected from the group consisting of eucalyptus and bamboo.
54. A genetically modified fungus, such as Apiospora, such as an Apiospora overexpressing the NRPS4, such as wherein the NRPS4 gene has been knocked out.
55. The genetically modified Apiospora according to item 54, wherein the mycelium of the genetically modified Apiospora does not repel water, such as when studied in a water droplet retention time study.
56. A host cell, such as a fungus, comprising the NRPS4 gene of Apiospora, with the proviso that the NRPS4 gene of Apiospora has been artificially introduced into the fungus.
57. A method of producing the compound according to any of items 15-37, wherein the method comprises:
   a. providing an NRPS gene from a fungus, such as the NRPS4 gene of Apiospora;
   b. inserting a promoter upstream of the NRPS gene and/or upstream of the NRPS cluster specific transcription factor, such as a constitutively active promoter, thereby providing an activated NRPS gene;
   c. introducing the activated NRPS gene into a suitable host cell, such as a fungus, such as Apiospora;
   d. growing the host cell under conditions that
      a. allows for the production of the enzyme encoded by the NRPS4 gene; and
      b. allows for the production of the compound;
   e. recovering the compound, such as by LC, HPLC.
58. The method according to any of the preceding items, wherein the compound is a compound according to any of the preceding items.
59. A method of selecting antigen-binding molecules with specificity towards an NRPS derived compound, the method comprising:
   a. Providing a library for selecting antigen-binding molecules, such as a library comprising phages;
   b. Providing a fungus expressing a compound as described in any of items items 15-37, and providing a purified version of the compound;
   c. Contacting the library with the fungus;
   d. Contacting the fungus with the purified version of the compound; and
   e. Retrieving antigen-binding molecules with specificity against the compound, such as retrieving phages with specificity against the compound.

1. A method of analysing and/or identifying one or more compounds produced by a fungus-derived Non-Ribosomal Peptide Synthetase (NRPS), wherein the method comprises:
a. providing a first fungus, such as Apiospora, and a second fungus of the same species and strain as the first fungus;
b. genetically modifying the expression of the NRPS in the first fungus, such as knocking out or overexpressing the NRPS gene, wherein the NRPS comprises a domain architecture wherein an epimerization (E) domain is present in alternating domains;
c. growing the first fungus and the second fungus under conditions suitable for the production of compounds, such as compounds of NRPS;
d. recovering extracts from the first fungus and the second fungus;
e. analysing the extracts; such as by LC-MS and/or NMR spectroscopy; and
f. comparing the analyses of the extracts between the first fungus and the second fungus, thereby identifying compounds of the NRPS.

2. The method according to claim 1, wherein the knocking out is achieved by gene editing, such as site-directed mutagenesis, such as the use of CRISPR/Cas9.

3. The method according to any of the preceding claims, wherein overexpression is achieved by inserting a promoter upstream of the NRPS gene and/or upstream of the NRPS cluster specific transcription factor, such as a constitutively active promoter.

4. The method according to any of the preceding claims, wherein the fungus is Apiospora, Aspergillus, Trichophyton, Candida, or Fusarium.

5. The method according to any of the preceding claims, wherein the fungus is Apiospora.

6. The method according to any of the preceding claims, wherein the Apiospora is Apiospora arundinis.

7. The method according to any of the preceding claims, wherein the Apiospora arundinis is Apiospora arundinis AAU773.

8. The method according to any of the preceding claims, wherein the fungus is not Neurospora crassa.

9. The method according to any of the preceding claims, wherein the fungus is able to repel water, such as in a water droplet retention time study.

10. The method according to any of the preceding claims, wherein the fungus is a mammalian disease-relevant fungus, such as a human disease-relevant fungus, such as a fungus selected from Aspergillus, Trichophyton, Candida, and Fusarium, preferably a species selected from, Trichophyton rubrum and Aspergillus Fumigatus,

11. The method according to any of the preceding claims, wherein the NRPS is encoded by the gene according to SEQ ID NO: 1, or a degenerate sequence thereof or by the cDNA according to SEQ ID NO: 2, or a degenerate sequence thereof, or the cDNA according to SEQ ID NO: 40, or a degenerate sequence thereof.

12. The method according to any of the preceding claims, wherein the NRPS is the protein according to SEQ ID NO: 4, or a variant thereof having at least 80% sequence identity, or the protein according to SEQ ID NO: 41, or a variant thereof having at least 80% sequence identity.

13. The method according to any of the preceding claims, wherein the compound is a secondary metabolite.

14. The method according to any of the preceding claims, wherein the method includes a step of identifying the NRPS by submitting the genome of said first and/or second fungus to antiSMASH for biosynthetic gene cluster prediction and/or to AUGUSTUS for gene prediction, preferably also comparing the domain architecture of the predicted NRPS genes to identify the NRPS displaying alternating epimerization domains.

15. An isolated compound identified by the method according to any of the preceding claims.

16. An isolated compound produced from the Non-Ribosomal Peptide Synthetase (NRPS).

17. The compound according to any of claims 15-16, wherein the compound is a secondary metabolite.

18. A compound, such as a secondary metabolite produced from a Non-Ribosomal Peptide Synthetase (NRPS) of a fungus, such as the Non-Ribosomal Peptide Synthetase 4 (NRPS4) of Apiospora.

19. The compound according to any of claims 15-18, wherein the compound comprises amino acids, such as proteinogenic amino acids and/or non-proteinogenic amino acids.

20. The compound according to any of claims 15-19, wherein the compound is further modified by at least one additional enzyme.

21. The compound according to any of claims 15-20, wherein the compound comprises a mixture of proteinogenic amino acids and non-proteinogenic amino.

22. The compound according to any of claims 19-21, wherein at least two of the amino acids, such as all amino acids, are coupled by peptide bonds formed between the α-nitrogen atom of one amino acid and the carbonyl carbon of a second amino acid.

23. The compound according to any of claims 15-22, wherein the compound is a peptide.

24. The compound according to any of claims 19-23, wherein the amino acids in the compound are positioned in a stereochemistry according to the general formula (L-D)ⁿ and/or (D-L)ⁿ, wherein n is an integer from 3-10, such as any of 3, 4, 5, 6, 7, 8, 9, or 10.

25. The compound according to any of claims 19-24, wherein each consecutive amino acid has sidechains in an opposing enantiomer in relation to the previous amino acid.

26. The compound according to any of claims 19-25, wherein the compound comprises a D/L alpha architecture in the backbone.

27. The compound according to any of claims 15-26, wherein the compound comprises 3-10 amino acids, such as any of 3 amino acids, such as 4 amino acids, such as 5 amino acids, such as 6 amino acids, such as 7 amino acids, such as 8 amino acids, such as 9 amino acids, or such as 10 amino acids, preferably such as 7 amino acids.

28. The compound according to any of claims 15-27, wherein the compound is cyclic.

29. The compound according to any of claims 15-27, wherein the compound is linear.

30. The compound according to any of claims 15-29, wherein the compound is a compound represented by Formula I-VI selected from: 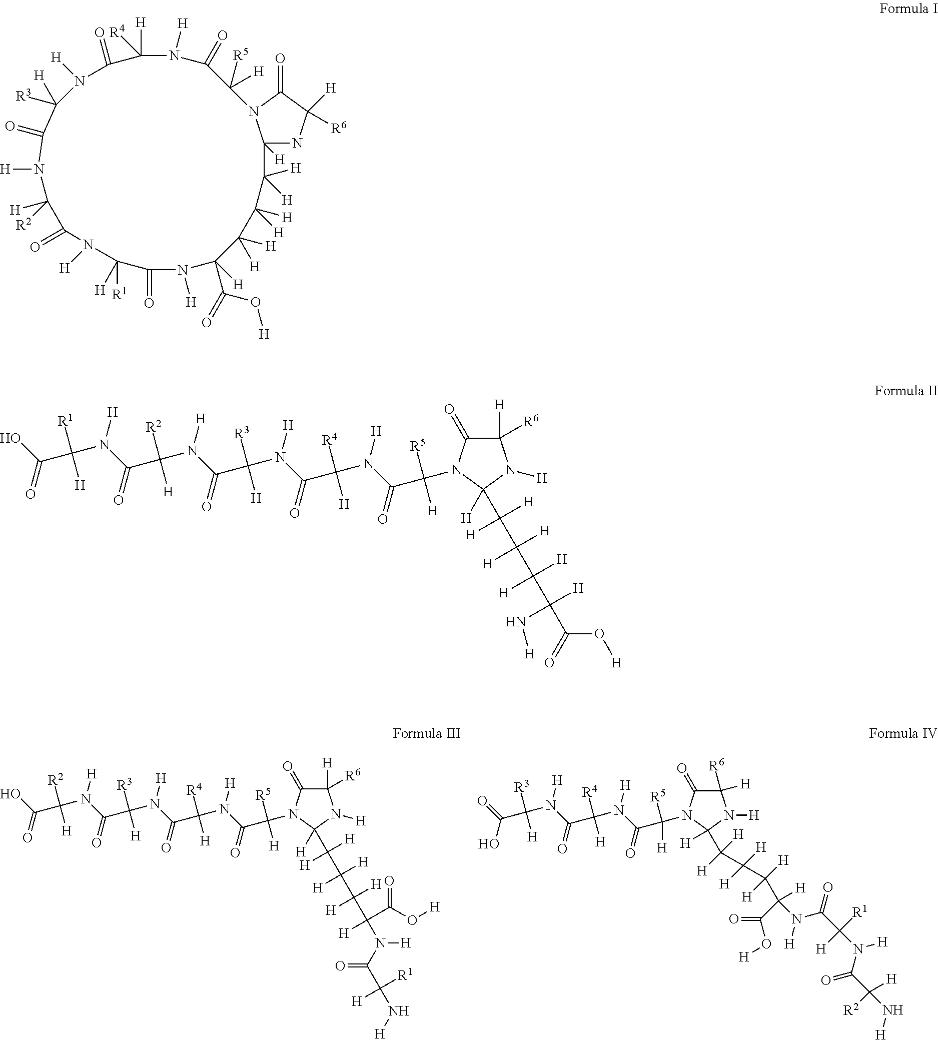 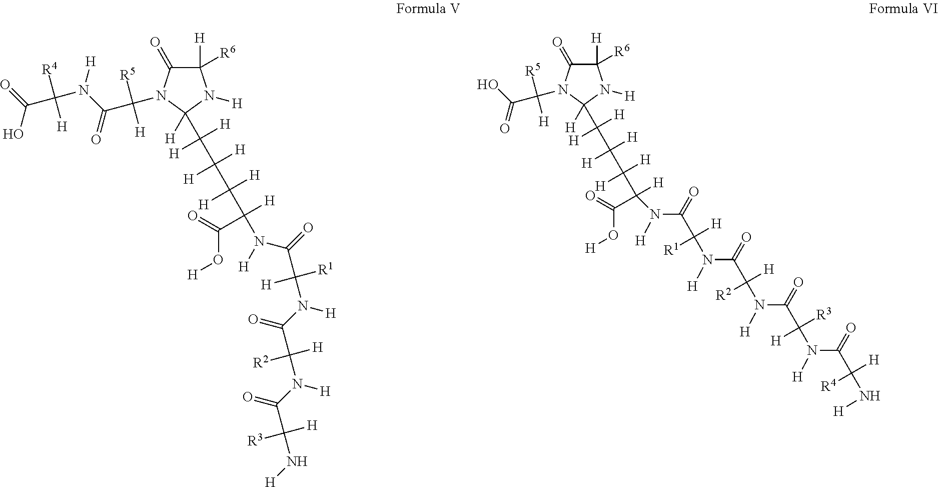
wherein R¹ is L-Tyrosine or D-Tyrosine; R² is L-Leucine or D-Leucine; R³ is L-Alanine, D-Alanine, L-Leucine or D-Leucine; R⁴ is L-Leucine or D-Leucine; R⁵ is L-Leucine or D-Leucine; and R⁶ is L-Leucine or D-Leucine, and the Lysine is a L-Lysine or a D-Lysine, with the proviso that when R¹, R³, R⁵ are L-enantiomers, R², R⁴, and R⁶ are D-enantiomers, or when R¹, R³, R⁵ are D-enantiomers, R², R⁴, and R⁶ are L-enantiomers.

31. The compound according to any of claims 15-30, wherein the compound is a salt thereof, such as a pharmaceutically acceptable salt thereof.

32. The compound according to any of claims 15-31, wherein the Apiospora is Apiospora arundinis.

33. The compound according to claim 32, wherein the Apiospora arundinis is Apiospora arundinis AAU773.

34. The compound according to any of claims 15-33, wherein the NRPS4 is encoded by the gene according to SEQ ID NO: 1, or a degenerate sequence thereof.

35. The method according to any of claims 15-33, wherein the NRPS4 is encoded by the cDNA according to SEQ ID NO: 2, or a degenerate sequence thereof, or the cDNA according to SEQ ID NO: 40, or a degenerate sequence thereof.

36. The compound according to any of claims 15-33, wherein the NRPS4 is the protein according to SEQ ID NO: 4, or a variant thereof having at least 80% sequence identity, or the protein according to SEQ ID NO: 41, or a variant thereof having at least 80% sequence identity.

37. The compound according to any of claims 15-36, wherein the compound is coupled to an adjuvant, such as a toxin.

38. The compound according to any of claims 15-37 for use as a vaccine.

39. The compound for use according to any of the preceding claims, wherein the vaccine is a vaccine against a fungus infection.

40. The compound for use according to any of claims 38-39, wherein the fungus infection is an infection with the fungus of which the compound is originally derived, such as where the NRPS is present in the genome of the fungus of which the compound is originally derived.

41. Use of the compound according to any of claims 15-36 as a surfactant.

42. An antigen-binding molecule having affinity against the compound according to any of claims 15-37, such as an antibody, or an antigen-binding fragment thereof.

43. An antigen-binding molecule having affinity against the extracellular surface of Apiospora, such as an antibody, or an antigen-binding fragment thereof.

44. The antigen-binding molecule according to any claims 42-43, wherein the extracellular surface of Apiospora is a compound according the any of the preceding claims, present on the surface of the fungus.

45. The antigen-binding molecule according to any claims 42-44, wherein the antigen-binding molecule can bind to a solubilized form of the compound, such as the not being bound to a cellular membrane, such as the membrane of a fungus.

46. The antigen-binding molecule according to any claims 42-45, wherein the antigen-binding molecule is a molecule selected from the group consisting of an antibody, a scFv, a single domain antibody (sdAb) or nanobody, a VHH, an isolated single variable domain, an affibody, a DARPin, a monobody, an anticalin, an affilin, an affimer type 1 molecule, an affimer type 2 molecule, an affitin, an alphabody, an anticalin, an avimer, a fynomer, a kunitz domain peptide, a nanoclamp, and an aptamer.

47. The antigen-binding molecule according to any claims 42-46, wherein the antigen-binding molecule comprises:
a heavy chain variable region comprising a CDR 1 region, a CDR 2 region, and a CDR 3 region; and
a light chain variable region comprising a CDR 1 region, a CDR 2 region, and a CDR 3 region.

48. The antigen-binding molecule according to 47, wherein said antigen-binding molecule is selected from the group consisting of:
a) an antigen-binding molecule comprising:
a CDR 1 region according to SEQ ID NO: 7, a CDR 2 region according to SEQ ID NO: 8 and a CDR 3 region according to SEQ ID NO: 9;
b) an antigen-binding molecule comprising:
a CDR 1 region according to SEQ ID NO: 12, a CDR 2 region according to SEQ ID NO: 13 and a CDR 3 region according to SEQ ID NO: 14;
c) an antigen-binding molecule comprising:
a CDR 1 region according to SEQ ID NO: 17, a CDR 2 region according to SEQ ID NO: 18 and a CDR 3 region according to SEQ ID NO: 19
d) an antigen-binding molecule comprising:
a CDR 1 region according to SEQ ID NO: 2, a CDR 2 region according to SEQ ID NO: 23 and a CDR 3 region according to SEQ ID NO: 24;
e) an antigen-binding molecule comprising:
a CDR 1 region according to SEQ ID NO: 27, a CDR 2 region according to SEQ ID NO: 28 and a CDR 3 region according to SEQ ID NO: 29;
f) an antigen-binding molecule comprising:
a CDR 1 region according to SEQ ID NO: 32, a CDR 2 region according to SEQ ID NO: 33 and a CDR 3 region according to SEQ ID NO: 34; or
g) an antigen-binding molecule comprising:
a CDR 1 region according to SEQ ID NO: 37, a CDR 2 region according to SEQ ID NO: 38 and a CDR 3 region according to SEQ ID NO: 39.

49. The antigen-binding molecule according to 48, wherein said antigen-binding molecules are single domain antibodies.

50. The antigen-binding molecule according to any of claims 42-49, wherein said antigen-binding molecule is selected from the group consisting of:
a) the single domain antibody according to SEQ ID NO: 6;
b) the single domain antibody according to SEQ ID NO: 11;
c) the single domain antibody according to SEQ ID NO: 16;
d) the single domain antibody according to SEQ ID NO: 21;
e) the single domain antibody according to SEQ ID NO: 26;
f) the single domain antibody according to SEQ ID NO: 31; or
g) the single domain antibody according to SEQ ID NO: 36.

51. Use of the antigen-binding molecule according to any claims 42-50 in a method of detecting Apiospora, such as an in vitro method.

52. The antigen-binding molecule according to any of claims 42-50 for use as a medicament.

53. The antigen-binding molecule according to any of claims 42-50 for use in the treatment of an infection with a mammalian disease-relevant fungus, such as a human disease-relevant fungus, such as a fungus selected from Aspergillus, Trichophyton, Candida, and Fusarium preferably a species selected from, Trichophyton rubrum and Aspergillus Fumigatus.

54. The antigen-binding molecule according to any of claims 42-50, wherein the antigen-binding molecule is an antibody-drug conjugate, such as coupled to a toxin or a medicament.

55. The antigen-binding molecule according to any of claims 42-50 or claim 54, wherein the antigen-binding molecule is coupled to a toxin.

56. Use of the antigen-binding molecule according to any of claims 42-50 or any of claims 54-55 in the treatment of a non-human infection, such as an infection on trees, such as a tree selected from the group consisting of eucalyptus and bamboo.

57. A genetically modified fungus, such as Apiospora, such as an Apiospora overexpressing the NRPS4, or wherein the NRPS gene, such as NRPS4 has been knocked out.

58. The genetically modified Apiospora according to claim 57, wherein the mycelium of the genetically modified fungus, such as Apiospora, does not repel water, such as when studied in a water droplet retention time study.

59. A host cell, such as a fungus, comprising the NRPS4 gene of Apiospora, with the proviso that the NRPS4 gene of Apiospora has been artificially introduced into the fungus.

60. A method of producing the compound according to any of claims 15-37, wherein the method comprises:
a. providing an NRPS gene from a fungus, such as the NRPS4 gene of Apiospora, such as the Pes1 gene of Aspergillus, such as the TERG_01444 gene of Trichophyton;
b. inserting a promoter upstream of the NRPS gene and/or upstream of the NRPS cluster specific transcription factor, such as a constitutively active promoter, thereby providing an activated NRPS gene;
c. introducing the activated NRPS gene into a suitable host cell, such as a fungus, such as Apiospora;
d. growing the host cell under conditions that
a. allows for the production of the enzyme encoded by the NRPS4 gene; and
b. allows for the production of the compound;
e. recovering the compound, such as by LC, HPLC.

61. The method according to claim 60, wherein the compound is a compound according to any of 15-37.

62. A method of selecting antigen-binding molecules with specificity towards an NRPS derived compound, the method comprising:
a. Providing a library for selecting antigen-binding molecules, such as a library comprising phages;
b. Providing a fungus expressing a compound as described in any of claims claims 15-37, and providing a purified version of the compound;
c. Contacting the library with the fungus;
d. Contacting the fungus with the purified version of the compound; and
e. Retrieving antigen-binding molecules with specificity against the compound, such as retrieving phages with specificity against the compound.
